(19) United States
(12) Patent Application Publication
(10) Pub. No.: US 2023/0381253 A1
(43) Pub. Date: Nov. 30, 2023

(54) MICROBES WITH REDUCED ADHESION CHARACTERISTICS

(71) Applicant: Wisconsin Alumni Research Foundation, Madison, WI (US)

(72) Inventors: Jan Peter van Pijkeren, Madison, WI (US); Laura Alexander, Fitchburg, WI (US)

(73) Assignee: Wisconsin Alumni Research Foundation, Madison, WI (US)

(21) Appl. No.: 18/323,912

(22) Filed: May 25, 2023

Related U.S. Application Data
(60) Provisional application No. 63/345,757, filed on May 25, 2022.

(51) Int. Cl. A61K 35/747; C12N 9/52; C12N 9/14; C07K 14/335

(52) U.S. Cl. CPC A61K 35/747; C12N 9/52; C12Y 304/2207; C12N 9/14; C07K 14/335

(57) ABSTRACT

Recombinant microorganisms and methods of using same. The recombinant microorganisms include one or more modifications that reduce the expression and/or activity of a sortase, a sortase-dependent protein, a fibronectin-binding protein, an autolysin, a surface-layer protein, an aggregation-promoting factor, and/or a collagen-binding protein. The modifications can reduce the adhesion characteristics with respect to the non-modified microbes. The recombinant microorganisms can further include a recombinant gene configured to express a biologic. The recombinant microorganisms can be used as delivery vehicles to deliver the biologics to sites such as the gastrointestinal tract.

### CROSS-REFERENCE TO RELATED APPLICATIONS

Priority is hereby claimed to provisional application Ser. No. 63/345,757, filed May 25, 2023, which is incorporated herein by reference.

### STATEMENT REGARDING FEDERALLY SPONSORED RESEARCH

This invention was made with government support under GM135483 awarded by the National Institutes of Health and 23-CRHF-0-6055 awarded by the USDA/NIFA. The government has certain rights in the invention.

### SEQUENCE LISTING

The instant application contains a Sequence Listing which has been submitted in XML format via EFS-Web and is hereby incorporated by reference in its entirety. The XML copy, created on May 12, 2023, is named Seq_List--P21008US02.xml and is 172,259 bytes in size.

### FIELD OF THE INVENTION

The invention is directed to microbes with reduced adhesion characteristics, such as adhesion to mucosal tissues.

### BACKGROUND

Bacteria engineered as therapeutic delivery vehicles are poised to become valuable tools for the future of personalized medicine. Bacteria can be engineered to produce recombinant effector molecules that would otherwise be difficult to manufacture and administer to treat specific diseases. Serving as both the production factory and delivery system of effector molecules, recombinant bacteria are a powerful chassis to deliver therapeutics following oral or intranasal administration. Several groups have engineered bacteria as delivery vehicles that demonstrated efficacy in various in vivo models, and some have recently advanced to human clinical trials (1-5).

An unresolved disadvantage posed by using recombinant bacteria as therapeutic delivery vehicles is the colonization risk. Persistence and colonization should be avoided to limit the delivery of high doses of certain therapeutics, which can have deleterious side effects. Excessive levels of therapeutic IL-22, for example, correlate with the development of psoriasis and the priming and proliferation of tumors (6-9). Unknown consequences might also correspond with the colonization of biotherapeutic delivering bacteria. These issues also present a challenge when developing probiotic bacteria as therapeutic delivery vehicles, as probiotic bacteria often exhibit the ability to associate with the epithelial layer of the gastrointestinal tract, which can lead to both stimulating the immune system and increasing the persistence of the probiotic in the gut (10-12). Therefore, biocontainment of biotherapeutic delivery vehicles to prevent these recombinant bacteria from proliferating in the host or external environment is ideal (13). Biocontainment systems developed in the synthetic biology industry include synthetic auxotrophy and ‘Deadman’ and ‘Passcode’ kill switches (14, 15). However, these systems only address the ability of recombinant microbes to replicate, not their ability to stimulate and interact with host tissue. Currently, the only method to eliminate microbes from complex communities is antibiotic treatment, which by itself also is likely to cause negative side effects by disrupting the resident gut microbiota (16).

Recombinant microbial biotherapeutic delivery vehicles with reduced colonization risk and enhanced biocontainment are needed.

### SUMMARY OF THE INVENTION

One aspect of the invention is directed to recombinant microorganisms. The recombinant microorganisms preferably comprise one or more modifications with respect to a corresponding microorganism not comprising the one or more modifications. The one or more modifications preferably reduce, in the recombinant microorganism with respect to the corresponding microorganism, expression and/or activity of one or more proteins expressed by the corresponding microorganism. The one or more proteins preferably comprise any one or more, any two or more, any three or more, any four or more, any five or more, any six or more, or each of a sortase, a sortase-dependent protein, a fibronectin-binding protein, an autolysin, a surface-layer protein, an aggregation-promoting factor, and a collagen-binding protein. The recombinant microorganism in some versions further comprises a recombinant gene configured to express a biologic, such as a therapeutic protein or RNA. The recombinant microorganism in some versions, is a member of Lactobacillales (a lactic acid bacteria), such as a member of Limosilactobacillus or Lactobacillus, such as L. reuteri.

Another aspect of the invention is directed to methods of administration. The methods preferably comprise administering the recombinant microorganism of the invention to a subject. The administering in some versions comprises orally administering the recombinant microorganism to the subject. The administering in some versions comprises introducing the recombinant microorganism to a gastrointestinal tract of the subject. The recombinant microorganism in some versions comprises a recombinant gene configured to express a biologic. The administering in some versions comprises introducing the biologic to a gastrointestinal tract of the subject.

The objects and advantages of the invention will appear more fully from the following detailed description of the preferred embodiment of the invention made in conjunction with the accompanying drawings.

### BRIEF DESCRIPTION OF THE DRAWINGS

FIG. 1. Adhesion protein mutant biocontainment concept. The locations of 10 putative adhesion proteins in the L. reuteri VPL1014 genome are indicated (left). We mutated each putative adhesion mutant individually and sequentially to yield a nonuple mutant. Depicted here are graphical examples of single mutants and the nonuple mutant (center). The nonuple mutant, lacking multiple adhesion proteins, has reduced adherence to human enteroid cells (FIG. 5D). In this graphic, wild-type L. reuteri can adhere to mucus or epithelial cells, while the nonuple mutant is unable to adhere (right).

FIGS. 2A-2D. Construction, recovery, and growth analysis of adhesin mutants. FIGS. 2A and 2B. The gene targets and recombineering oligonucleotides used in the examples are listed on the left. DNA sequences of the targeted adhesion proteins of L. reuteri are shown aligned with each recombineering oligonucleotide, with the encoded amino acid listed above. Numbers above the first and last amino acid encoded by each sequence indicate the amino acid positions. The directions of the lagging strands are indicated, and underneath are the mismatches in the recombineering oligonucleotides, which result in internal stop codons in each gene. The resulting amino acid mutations are indicated underneath the mismatched nucleotides. The coding strand is indicated in the third column, and barcodes derived from oVPL3848 are listed in the fourth column. FIG. 2C. Scheme to optimize adhesin mutant recovery. Barcoding oligonucleotide oVPL3848 (3848, black) was dual-transformed into VPL4011 with three oligonucleotides targeting different adhesins (####; red, yellow, and blue) via electroporation. Transformants were selected on agar supplemented with chloramphenicol (Cm). Thirty CFU from each transformation were screened for mutant genotypes via MAMA PCR on a 96-well plate. In our design, wild-type genotypes have an expected size of 1 kb, while mutant genotypes should be 0.5 kb. If no mutants were recovered from one of the transformations (yellow, in this example), the corresponding recombineering oligonucleotide was re-transformed alongside oligonucleotides targeting two additional adhesins. This process was repeated until each adhesin mutant was recovered. FIG. 2D. Growth curves of all single mutants. Wild-type control (WT) is VPL4052, which contains the cat* gene insertion restored to cat with oVPL283. Data for WT is the same across the three growth curves, while the mutants are split across the three. The results shown are averages from three independent experiments±standard error of the mean.

FIGS. 3A-3H. Adhesin mutant gastrointestinal survival in mice and adhesion to human colon cancer cells. FIG. 3A. Mice (n=5-8) were administered with 108 CFU of each mutant for two consecutive days. At 15 h, 27 h, and 39 h after the second gavage, fecal material was collected, resuspended to 100 mg/mL in PBS, and plated for quantification. FIG. 3B. At 15 h, we measured the survival of each adhesion mutant following transit through the murine GI tract (n=5/group). A mix of VPL4011 (n=8) transformed with an oligonucleotide conferring each mutant barcode served as a control (WT mix). FIG. 3C. Persistence of each mutant is depicted as the CFU recovered over the course of the in vivo experiment. Data for WT is the same across both graphs. FIG. 3D. Percent adhesion of each mutant to HT-29 cells was compared to wild-type. VPL4052 served as the WT control. The results shown are averages from six independent experiments with three technical replicates each, ±standard error of the mean, ns=no statistical difference. FIG. 3E. Percent adhesion of each mutant and complements thereof to HT-29 cells were compared to wild-type. VPL4052 served as the WT control. The results shown are averages from six independent experiments with three technical replicates each, ±standard error of the mean, ns=no statistical difference. FIGS. 3F-3H. Change in relative ratio of each strain within each sample recovered from enteroid monolayers (TF) compared to the respective ratio of each strain in the starting mixture (T0). Data is presented as the change in relative percent (Δ%(TF−T0) for the barcode control mix (FIG. 3F), mutant mix (FIG. 3G), and the complemented mix (FIG. 3H) based on sequencing reads targeting the cat barcode. Positive numbers indicate an increase in relative ratio while negative numbers indicate a decrease. The results shown are averages from three independent experiments with three technical replicates each, ±standard error of the mean; *, p<0.05. For FIGS. 3E-3F, the first of the two bars shown for each strain is a not-washed condition, and the second of the two bars shown for each strain is a washed condition.

FIG. 4. Adhesion competition ratios for individual mutants and complemented mutants in an HT-29 adhesion competition assay. Adhesion competition ratios were calculated as the ratio of the mutant or the complemented mutant (all chloramphenicol-resistant) to the wild-type strain.

FIGS. 5A-5D. Nonuple growth, phage production, and adhesion to human colon-cancer cells. FIG. 5A. Growth curve and mitomycin C induction of VPL1014 (WT) and nonuple mutant. FIG. 5B. At the endpoint of the growth experiment (T8), samples derived from uninduced and induced cultures of VPL1014 (WT) and the nonuple variant were processed to quantify phage production (PFU) (p>0.3). FIG. 5C. Adhesion of VPL1014 (WT) and nonuple mutant to monolayers of HT-29 cells (p>0.4). FIG. 5D. Adhesion competition experiment on human enteroid monolayers between wild-type control (VPL4216 (L. reuteri::rpoB(H488R)) and nonuple mutant. Multiplicities of infection (MOI) ratios of 5:1 and 30:1 were tested. Results are expressed as a ratio of wild-type CFU recovered compared to nonuple CFU recovered from the adhesion assay. T0 and TF represent the ratio of WT and nonuple cells before and after the adhesion assay, respectively. For FIGS. 5A and 5B, the results shown are averages from three independent experiments±standard error of the mean. For FIGS. 5C and 5D, the results shown are averages from three independent experiments (with three technical replicates each)±standard error of the mean. *, p<0.05; ***, p<0.005.

FIGS. 6A-6B. IL-22 release from Non-IL22. FIG. 6A. Phage-mediated release of IL-22 by LR-IL22 or Non-IL22 induced with mitomycin C in vitro, as detected by ELISA. Values are expressed as the percent of IL-22 detected in the supernatant compared to total IL-22 detected in the cell pellet and supernatant combined. Results shown are averages from three independent experiments±standard error of the mean. FIG. 6B. Total IL-22 produced (supernatant+cell lysate) by LR-IL22 and Non-IL22 by cultures induced with mitomycin C. Samples were harvested five-hours post-induction, and were detected by ELISA. *, p<0.05.

FIG. 7. In vivo survival of Non-IL22 in obese mice fed high-fat, high-sugar diet Mice were fed a high-fat diet for 8 weeks before and throughout treatment, and body weight was measured every two weeks. At week 9, treatment began by administering PBS (sham) or 10⁹ CFU of bacteria of WT-Ctl, Non-Ctl, WT-IL22, or Non-IL22 (n=11-12/group). Fecal material was sampled for bacterial CFU counts every two weeks during treatment. All tissue and blood samples were collected at the end point (16 weeks). FIG. 7 shows the average bacterial CFU recovered on agar plates supplemented with rifampicin across all samplings. No CFU was recovered from the sham-treated group.

### DETAILED DESCRIPTION OF THE INVENTION

One aspect of the invention is directed to recombinant microorganisms. The recombinant microorganisms comprise one or more modifications with respect to a corresponding microorganism not comprising the one or more modifications. “Recombinant microorganism” refers to a microorganism that comprises a recombinant nucleic acid, a recombinant gene, or a recombinant polypeptide. A recombinant nucleic acid or polypeptide is one comprising a sequence that is not naturally occurring. A recombinant gene is a gene that comprises a recombinant nucleic acid sequence, is present within a microorganism in which it does not naturally occur, and/or is present at a locus (e.g., genetic locus or on an extrachromosomal plasmid) that is different than the locus in which it is present in a corresponding native microorganism.

The one or more modifications in the recombinant microorganisms preferably include modifications that reduce, in the recombinant microorganism with respect to the corresponding microorganism, the expression and/or activity of one or more proteins expressed by the corresponding microorganism. “Reduce” in this context refers to any diminishment or complete ablation of the expression and/or activity of the protein in the recombinant microorganism with respect to the corresponding microorganism. Thus, a reduction of the expression and/or activity of a protein in this context encompass its complete absence in the recombinant microorganism, such as by deletion of the protein's gene or other mechanisms. “Expression” used with respect to a protein refers to the production of the protein. Such expression can comprise translation of mRNA encoding the protein and, ultimately, transcription of genomic DNA into mRNA. “Activity” used with respect to a protein broadly encompasses any particular activity of the protein within the cell, such that a reduction of any one or more particular activities constitutes a reduction of the activity of the protein generally. Preferred activities that can be reduced include the activities of the proteins described herein.

The one or more proteins in the corresponding microorganisms whose expression or activity is reduced by the modifications to the recombinant cells can comprise any one or more, any two or more, any three or more, any four or more, any five or more, any six or more, or each of a sortase, a sortase-dependent protein, a fibronectin-binding protein, an autolysin, a surface-layer protein, an aggregation-promoting factor, and a collagen-binding protein, in any combination.

Sortases are a class of microbial enzymes that modify surface proteins by recognizing and cleaving a carboxyl-terminal sorting signal. For some substrates of sortase enzymes, the recognition signal comprises the motif LPxTG (Leu-Pro-any-Thr-Gly), followed by a highly hydrophobic transmembrane sequence and subsequently followed by a cluster of basic residues such as arginine, wherein cleavage occurs between the Thr and Gly, with transient attachment through the Thr residue to the active site Cys residue, followed by transpeptidation that attaches the protein covalently to cell wall components. Sortases occur in almost all Gram-positive bacteria and some Gram-negative bacteria (e.g. Shewanella putrefaciens) and Archaea (e.g. Methanobacterium thermoautotrophicum. Sortases and their activities are well known in the art. See, e.g., Spirig et al. 2011 (Spirig T, Weiner E M, Clubb R T. Sortase enzymes in Gram-positive bacteria. Mol Microbiol. 2011 December; 82(5):1044-59). Exemplary sortases whose expression or activity can be reduced in accordance with the invention include the sortases discussed in the references cited herein or discussed elsewhere herein, and homologs thereof. Other exemplary sortases whose expression or activity can be reduced in accordance with the invention include sortase A of Limosilactobacillus reuteri comprising the amino acid sequence of SEQ ID NO:2, and homologs thereof comprising amino acid sequences at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to SEQ ID NO:2. An exemplary coding sequence for SEQ ID NO:2 is SEQ ID NO:1.

Sortase-dependent proteins are a class of microbial proteins that facilitate adhesion and nutrient acquisition (Jeya M, Lee K-M, Tiwari M K, Kim J-S, Gunasekaran P, Kim S-Y, Kim I-W, Lee J-K. 2009. Isolation of a novel high erythritol-producing Pseudozyma tsukubaensis and scale-up of erythritol fermentation to industrial level. Appl Microbiol Biotechnol 83:225-231). SDPs are surface-associated proteins that are covalently coupled to the cell wall by the sortase enzyme (e.g., SrtA) (Zhu F, Lu L, Fu S, Zhong X, Hu M, Deng Z, Liu T. 2015. Targeted engineering and scale up of lycopene overproduction in Escherichia coli. Process Biochemistry 50:341-346). Sortase-dependent proteins exhibit a conserved molecular structure that includes an N-terminal signal peptide that directs sortase-dependent proteins to surface localization (Wang G, Haringa C, Noorman H, Chu J, Zhuang Y. 2020. Developing a computational framework to advance bioprocess scale-up. Trends in Biotechnology 38:846-856), a C-terminal LPxTG motif that anchors sortase-dependent proteins to the cell wall (Fage C, Lemire N, Moineau S. 2021. Delivery of CRISPR-Cas systems using phage-based vectors. Current Opinion in Biotechnology 68:174-180) (Jeya et al. 2009), a C-terminal transmembrane helix, and a positively charged tail (Fage et al. 2021 and Jeya et al. 2009). Sortase-dependent proteins and their activities are well known in the art. See, e.g., Banla et al. 2019 (Banla L I, Pickrum A M, Hayward M, Kristich C J, Salzman N H. Sortase-Dependent Proteins Promote Gastrointestinal Colonization by Enterococci. Infect Immun. 2019 Apr. 23; 87(5):e00853-18). Exemplary sortase-dependent proteins whose expression or activity can be reduced in accordance with the invention include the sortase-dependent proteins discussed in the references cited herein or discussed elsewhere herein, and homologs thereof. Other exemplary sortase-dependent proteins whose expression or activity can be reduced in accordance with the invention include the sortase-dependent proteins of Limosilactobacillus reuteri comprising the amino acid sequences of SEQ ID NOS:4, 6, 8, 10, and 12, and homologs thereof comprising amino acid sequences at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to any one of SEQ ID NOS:4, 6, 8, 10, and 12. Exemplary coding sequences for SEQ ID NOS:4, 6, 8, 10, and 12 are SEQ ID NOS:3, 5, 7, 9, and 11, respectively.

Fibronectin-binding proteins are a class of microbial proteins that bind fibronectin. Fibronectin-binding proteins are well known in the art (Hymes J P, Klaenhammer T R. Stuck in the Middle: Fibronectin-Binding Proteins in Gram-Positive Bacteria. Front Microbiol. 2016 Sep. 22; 7:1504) (Speziale P, Pietrocola G. The Multivalent Role of Fibronectin-Binding Proteins A and B (FnBPA and FnBPB) of Staphylococcus aureus in Host Infections. Front Microbiol. 2020 Aug. 26; 11:2054). Fbp54 is an exemplary fibronectin-binding protein whose expression or activity can be reduced in accordance with the invention. Fbp54 is found across a variety of host-associated commensals, as well as the probiotic species Lactobacillus acidophilus, L. casei, L. plantarum, L. brevis, L. rhamnosus, and Bacillus subtilis (Altermann E., Russell W. M., Azcarate-Peril M. A., Barrangou R., Buck B. L., McAuliffe O., et al. (2005). Complete genome sequence of the probiotic lactic acid bacterium Lactobacillus acidophilus NCFM. Proc. Natl. Acad. Sci. U.S.A. 102 3906-3912) (Boekhorst J., Wels M., Kleerebezem M., Siezen R. J. (2006). The predicted secretome of Lactobacillus plantarum WCFS1 sheds light on interactions with its environment. Microbiology 152(Pt 11) 3175-3183) (Vélez MP, De Keersmaecker S C, Vanderleyden J. Adherence factors of Lactobacillus in the human gastrointestinal tract. FEMS Microbiol Lett. 2007 November; 276(2):140-8) (Munoz-Provencio D., Perez-Martinez G., Monedero V. (2010). Characterization of a fibronectin-binding protein from Lactobacillus casei BL23. J. Appl. Microbiol. 108 1050-1059). Other exemplary fibronectin-binding proteins whose expression or activity can be reduced in accordance with the invention include any fibronectin-binding proteins discussed in the references cited herein or discussed elsewhere herein, and homologs thereof. Other exemplary fibronectin-binding proteins whose expression or activity can be reduced in accordance with the invention include the fibronectin-binding protein of Limosilactobacillus reuteri comprising the amino acid sequence of SEQ ID NO:14, and homologs thereof comprising amino acid sequences at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to SEQ ID NO:14. An exemplary coding sequence for SEQ ID NO:14 is SEQ ID NO:13.

Autolysins are a class of endogenous microbial lytic enzymes that break down the peptidoglycan components of biological cells (Jaenicke L, Kuhne W, Spessert R, Wahle U, Waffenschmidt S. Cell-wall lytic enzymes (autolysins) of Chlamydomonas reinhardtii are (hydroxy)proline-specific proteases. Eur J Biochem. 1987 Dec. 30; 170(1-2):485-91) (Buchanan M J, Imam S H, Eskue W A, Snell W J. Activation of the cell wall degrading protease, lysin, during sexual signalling in Chlamydomonas: the enzyme is stored as an inactive, higher relative molecular mass precursor in the periplasm. J Cell Biol. 1989 January; 108(1):199-207) (Matsuda Y (1998). “Gametolysin”. In Barrett A J, Rawlings N D, Woessner J F (eds.). Handbook of Proteolytic Enzymes. London: Academic Press. pp. 1140-1143) (Clarke A J. The “hole” story of predatory outer-membrane vesicles. Can J Microbiol. 2018 September; 64(9):589-599) (Porayath C, Suresh M K, Biswas R, Nair B G, Mishra N, Pal S. Autolysin mediated adherence of Staphylococcus aureus with Fibronectin, Gelatin and Heparin. Int J Biol Macromol. 2018 Apr. 15; 110:179-184). Amidases (EC 3.5.1.28), gametolysins (EC 3.4.24.38), and glucosaminidases are types of autolysins (Clarke et al. 2018) (Smith T J, Blackman S A, Foster S J. Autolysins of Bacillus subtilis: multiple enzymes with multiple functions. Microbiology (Reading). 2000 February; 146 (Pt 2):249-262). Exemplary autolysins whose expression or activity can be reduced in accordance with the invention include any autolysins discussed in the references cited herein or discussed elsewhere herein, and homologs thereof. Other exemplary autolysins whose expression or activity can be reduced in accordance with the invention include the autolysin of Limosilactobacillus reuteri comprising the amino acid sequence of SEQ ID NO:16, and homologs thereof comprising amino acid sequences at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to SEQ ID NO:16. An exemplary coding sequence for SEQ ID NO:16 is SEQ ID NO:15.

Surface-layer proteins are a class of highly expressed and ubiquitous microbial proteins that self-assemble on the outside of bacteria and archaea to form crystalline protein coats (J. H. Y. Lau, J. F. Nomellini, J. Smit, Analysis of high-level S-layer protein secretion in Caulobacter crescentus. Can. J. Microbiol. 56, 501-514 (2010)) (D. Pum, J. L. Toca-Herrera, U. B. Sleytr, S-layer protein self-assembly. Int. J. Mol. Sci. 14, 2484-2501 (2013)) (C. Zhu et al., Diversity in S-layers. Prog. Biophys. Mol. Biol. 123, 1-15 (2017)). Surface-layer proteins undergo a phase transition from aqueous to solid as part of their biological assembly and function (E. Baranova et al., SbsB structure and lattice reconstruction unveil Ca2+ triggered S-layer assembly. Nature 487, 119-122 (2012)) (J. Herrmann et al., Environmental calcium controls alternate physical states of the Caulobacter surface layer. Biophys. J. 112, 1841-1851 (2017)). Exemplary surface-layer proteins whose expression or activity can be reduced in accordance with the invention include any surface-layer proteins discussed in the references cited herein or discussed elsewhere herein, and homologs thereof. Other exemplary surface-layer proteins whose expression or activity can be reduced in accordance with the invention include the surface-layer protein of Limosilactobacillus reuteri comprising the amino acid sequence of SEQ ID NO:18, and homologs thereof comprising amino acid sequences at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to SEQ ID NO:18. An exemplary coding sequence for SEQ ID NO:18 is SEQ ID NO:17.

Aggregation-promoting factors are a call of microbial cell-surface proteins that facilitate microbial aggregation (including autoaggregation) and biofilm accumulation (Kmet V, Callegari M L, Bottazzi V, and Morelli L. 1995. Aggregation-promoting factor in pig intestinal Lactobacillus strains. Lett. Appl. Microbiol. 21:351-353.) (Kmet V, Lucchini F. Aggregation-promoting factor in human vaginal Lactobacillus strains. FEMS Immunol Med Microbiol. 1997 October; 19(2):111-4) (Goh Y J and Klaenhammer T R. 2010. Functional roles of aggregation-promoting-like factor in stress tolerance and adherence of Lactobacillus acidophilus NCFM. Appl. Environ. Microbiol. 76:5005-5012) (Reniero R, Cocconcelli P, Bottazzi V, and Morelli L. 1992. High-frequency of conjugation in Lactobacillus mediated by an aggregation-promoting factor. J. Gen. Microbiol. 138:763-768) (Boris S, Suarez J E, and Barbes C. 1997. Characterization of the aggregation promoting factor from Lactobacillus gasseri, a vaginal isolate. J. Appl. Microbiol. 83:413-420) (Lozo J, Jovcic B, Kojic M, Dalgalarrondo M, Chobert J M, Haertle T, and Topisirovic L. 2007. Molecular characterization of a novel bacteriocin and an unusually large aggregation factor of Lactobacillus paracasei subsp. paracasei BGSJ2-8, a natural isolate from homemade cheese. Curr. Microbiol. 55:266-271) (Schroeder K, Jularic M, Horsburgh S M, Hirschhausen N, Neumann C, Bertling A, Schulte A, Foster S, Kehrel B E, Peters G, and Heilmann C. 2009. Molecular characterization of a novel Staphylococcus aureus surface protein (SasC) involved in cell aggregation and biofilm accumulation. PLoS One 4:e7567) (Marcotte H, Ferrari S, Cesena C, Hammarstrom L, Morelli L, Pozzi G, and Oggioni M R. 2004. The aggregation-promoting factor of Lactobacillus crispatus M247 and its genetic locus. J. Appl. Microbiol. 97:749-756) (Siciliano R A, Cacace G, Mazzeo M F, Morelli L, Elli M, Rossi M, and Malorni A. 2008. Proteomic investigation of the aggregation phenomenon in Lactobacillus crispatus. Biochim. Biophys. Acta 1784:335-342) (Shibata Y, Hiratsuka K, Hayakawa M, Shiroza T, Takiguchi H, Nagatsuka Y, and Abiko Y. 2003. A 35-kDa co-aggregation factor is a hemin binding protein in Porphyromonas gingivalis. Biochem. Biophys. Res. Commun. 300:351-356) (Jankovic I, Ventura M, Meylan V, Rouvet M, Elli M, and Zink R. 2003. Contribution of aggregation-promoting factor to maintenance of cell shape in Lactobacillus gasseri 4B2. J. Bacteriol. 185:3288-3296). Aggregation-promoting factor genes are highly conserved genes that display their maximum expression rates at the stationary phase of growth (Goh et al. 2010) and that typically code for extracellular proteins that range in size from about 260 to 330 amino acids (Ventura M, Jankovic I, Walker D C, Pridmore R D, and Zink R. 2002. Identification and characterization of novel surface proteins in Lactobacillus johnsonii and Lactobacillus gasseri. Appl. Environ. Microbiol. 68:6172-6181). Exemplary aggregation-promoting factors whose expression or activity can be reduced in accordance with the invention include any aggregation-promoting factors discussed in the references cited herein or discussed elsewhere herein, and homologs thereof. Other exemplary aggregation-promoting factors whose expression or activity can be reduced in accordance with the invention include the aggregation-promoting factor of Limosilactobacillus reuteri comprising the amino acid sequence of SEQ ID NO:20, and homologs thereof comprising amino acid sequences at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to SEQ ID NO:20. An exemplary coding sequence for SEQ ID NO:20 is SEQ ID NO:19.

Collagen-binding proteins are a class of microbial proteins that bind directly to collagen. Collagen-binding proteins are well known in the art, as are various collagen types (Types I-XXVII). See Arora et al. 2021 (Arora S, Gordon J, Hook M. Collagen Binding Proteins of Gram-Positive Pathogens. Front Microbiol. 2021 Feb. 5; 12:628798) and Farndale et al. 2019 (Farndale R W. Collagen-binding proteins: insights from the Collagen Toolkits. Essays Biochem. 2019 Sep. 13; 63(3):337-348). Exemplary collagen-binding proteins whose expression or activity can be reduced in accordance with the invention include CnBP of Limosilactobacillus reuteri (Hsueh H-Y, Yueh P-Y, Yu B, Zhao X, Liu J-R. 2010. Expression of Lactobacillus reuteri Pg4 collagen-binding protein gene in Lactobacillus casei ATCC 393 increases its adhesion ability to Caco-2 cells. J Agric Food Chem 58:12182-12191) (Miyoshi Y, Okada S, Uchimura T, Satoh E. 2006. A mucus adhesion promoting protein, MapA, mediates the adhesion of Lactobacillus reuteri to Caco-2 human intestinal epithelial cells. Bioscience, Biotechnology, and Biochemistry 70:1622-1628), Ace of Enterococcus faecalis (Rich, R. L., Kreikemeyer, B., Owens, R. T., LaBrenz, S., Narayana, S. V., Weinstock, G. M., et al. (1999). Ace is a collagen-binding MSCRAMM from Enterococcus faecalis. J. Biol. Chem. 274, 26939-26945), CNA of Staphylococcus aureus (Speziale, P., Raucci, G., Visai, L., Switalski, L. M., Timpl, R., and Hook, M. (1986). Binding of collagen to Staphylococcus aureus Cowan 1. J. Bacteriol. 167, 77-81), Acm of Enterococcus faecium (Nallapareddy, S. R., Weinstock, G. M., and Murray, B. E. (2003). Clinical isolates of Enterococcus faecium exhibit strain-specific collagen binding mediated by Acm, a new member of the MSCRAMM family. Mol. Microbiol. 47, 1733-1747), Cnm of Streptococcus mutans (Sato, Y., Okamoto, K., Kagami, A., Yamamoto, Y., Igarashi, T., and Kizaki, H. (2004). Streptococcus mutans strains harboring collagen-binding adhesin. J. Dent. Res. 83, 534-539), Cne of Streptococcus equi (Lannergard, J., Frykberg, L., and Guss, B. (2003). CNE, a collagen-binding protein of Streptococcus equi. FEMS Microbiol. Lett. 222, 69-74), and Cbm of S. mutans (Nomura, R., Nakano, K., Naka, S., Nemoto, H., Masuda, K., Lapirattanakul, J., et al. (2012). Identification and characterization of a collagen-binding protein, Cbm, in Streptococcus mutans. Mol. Oral Microbiol. 27, 308-323), among others (Arora et al. 2021, Farndale et al. 2019); any other collagen-binding proteins discussed in the references cited herein or discussed elsewhere herein, and homologs thereof. Other exemplary collagen-binding proteins whose expression or activity can be reduced in accordance with the invention include the collagen-binding protein of Limosilactobacillus reuteri comprising the amino acid sequence of SEQ ID NO:22, and homologs thereof comprising amino acid sequences at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to SEQ ID NO:22. An exemplary coding sequence for SEQ ID NO:22 is SEQ ID NO:21.

Modifications that reduce the expression or activity of a protein include any modification to a microorganism that ablates, reduces, inhibits, or otherwise disrupts production of the protein, renders the protein non-functional, or otherwise reduces or ablates the protein's activity. Accordingly, in some instances, production of a protein can be completely shut down, such as by partially or completely deleting the gene of the protein. As used herein, “gene” minimally refers to a promoter operationally linked to a coding sequence. A gene can optionally include other genetic elements that facilitate or regulate transcription and/or translation of the coding sequence. Such genetic elements can include enhancers ribosome and binding sites (RBSs), among other elements.

There are many well-known ways to reduce the expression or activity of a protein. This can be accomplished, for example, by introducing one or more genetic modifications. As used herein, “genetic modifications” refer to any differences in the nucleic acid composition of a cell, whether in the cell's native chromosome or in endogenous or exogenous non-chromosomal plasmids harbored within the cell. Examples of genetic modifications that reduce the expression or activity of a protein include but are not limited to substitutions, partial or complete deletions, insertions, or other variations to the gene of the protein. These include substitutions, partial or complete deletions, insertions, or other variations of the protein's coding sequence or a promoter of the coding sequence. In some versions, a gene or coding sequence can be partially or completely replaced with a selection marker or screenable marker. In some versions, the genetic modifications can include the introduction of constructs that express ribozymes or antisense sequences that target the mRNA of the gene of the protein. Various other genetic modifications that reduce the activity of a gene or gene product are described elsewhere herein. Various methods for introducing genetic modifications are well known in the art and include homologous recombination, among other mechanisms. See, e.g., Green et al., Molecular Cloning: A laboratory manual, 4^{th} ed., Cold Spring Harbor Laboratory Press (2012) and Sambrook et al., Molecular Cloning: A Laboratory Manual, 3^{rd} ed., Cold Spring Harbor Laboratory Press (2001).

In some versions of the invention, the recombinant microorganism exhibits a growth rate during exponential phase of growth no less than 40%, no less than 45%, no less than 50%, no less than 55%, no less than 60%, no less than 65%, no less than 70%, no less than 75%, no less than 80%, no less than 85%, no less than 90%, no less than 91%, no less than 92%, no less than 93%, no less than 94%, no less than 95%, no less than 96%, no less than 97%, no less than 98%, or no less than 99% of a growth rate exhibited by the corresponding microorganism during exponential phase of growth, wherein growth rate (r) is determined by the formula r=(ln [OD2/OD1])/(T2−T1), OD is OD600, T is time, OD1 is OD600 at time 1, and OD2 is OD600 at time 2.

In some versions, the recombinant microorganism comprises a recombinant gene configured to express a biologic. The recombinant gene can comprise a coding sequence of the biologic operably linked to a promoter. The promoter can be heterologous to the coding sequence. In some versions, the promoter is a constitutive promoter. In some versions, the promoter is an inducible promoter.

As used herein, “biologic” refers to any biologically active product capable of being expressed from a gene. The biologic can be biologically active in vivo in any prokaryote or eukaryote or in vitro in any in vitro biochemical system. The biologic can have any activity, whether enzymatic, binding, structural, etc. Biologics that have a therapeutic effect activity are referred to herein as “therapeutic biologics.” Therapeutic biologics can target and promote growth of beneficial cells in the subject, target and inhibit growth of deleterious cells in the subject, target certain cells for destruction, or can have any other activity that provides a therapeutic effect to a subject to which they are introduced.

Examples of biologics include nucleic acids and polypeptides.

Exemplary nucleic acid biologics include DNA and RNA. Preferred nucleic acid biologics include therapeutic nucleic acids. Nucleic acid biologics can generally be classified as nucleotides and nucleosides, oligonucleotides, or polynucleotides. Various types of nucleic acid biologics include oligonucleotides for antisense and antigene applications, DNA aptamers, antisense oligodeoxynucleotides, DNAzymes, DNA vaccines, RNA-based therapeutics, RNA aptamers, RNA Decoys, antisense RNA, ribozymes, small interfering RNAs, and microRNAs, among others.

Suitable polypeptide biologics can include any polypeptide of interest. The polypeptide can have any of a number of amino acid chain lengths. In some versions, the polypeptide can have an amino acid chain length of from about 2 to about 2,000 amino acids, from about 2 to about 1,000 amino acids, from about 2 to about 500 amino acids, from about 3 to about 250 amino acids, or from about 3 to about 225 amino acids. The polypeptide can have a net positive charge at neutral pH, a net negative charge at neutral pH, or a net neutral charge at neutral pH. The polypeptide is preferably soluble in water. The polypeptide can form a globular or fibrous structure or can have an intrinsically disordered structure.

The polypeptide can have any of a number of functionalities. The polypeptide, for example, can be enzymatic or non-enzymatic. The polypeptide can be fluorescent or non-fluorescent. The polypeptide can be a cytokine, a hormone, an antibody, an antimicrobial peptide, and an antigenic peptide, among others.

Exemplary classes of cytokines include interleukins, lymphokines, monokines, interferons (IFNs), colony stimulating factors (CSFs), among others. Specific exemplary cytokines include IL-1 alpha (IL1a), IL-1 beta (IL1b), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, IL-36, IFN-alpha, IFN-beta IFN-gamma, TNF-alpha, TNF-beta, CNTF (C-NTF), LIF, OSM (oncostatin-M), EPO (erythropoietin), G-CSF (GCSF), GM-CSF (GMCSF), M-CSF (MCSF), SCF, GH (growth hormone), PRL (prolactin), aFGF (FGF-acidic), bFGF (FGF-basic), INT-2, KGF (FGF7). EGF, TGF-alpha, TGF-beta, PDGF, betacellulin (BTC), SCDGF, amphiregulin, and HB-EG, among others.

Exemplary hormones include epinephrine, melatonin, triiodothyronine, thyroxine, amylin (or islet amyloid polypeptide), adiponectin, adrenocorticotropic hormone (or corticotropin), angiotensinogen, angiotensin, antidiuretic hormone (or vasopressin, arginine vasopressin), atrial-natriuretic peptide (or atriopeptin), brain natriuretic peptide, calcitonin, cholecystokinin, corticotropin-releasing hormone, cortistatin, encephalin, endothelin, erythropoietin, follicle-stimulating hormone, galanin, gastric inhibitory polypeptide, gastrin, ghrelin, glucagon, glucagon-like peptide-1, gonadotropin-releasing hormone, growth hormone-releasing hormone, hepcidin, human chorionic gonadotropin, human placental lactogen, growth hormone, inhibin, insulin, insulin-like growth factor (or somatomedin), leptin, lipotropin, luteinizing hormone, melanocyte stimulating hormone, motilin, orexin, oxytocin, pancreatic polypeptide, parathyroid hormone, pituitary adenylate cyclase-activating peptide, prolactin, prolactin releasing hormone, relaxin, renin, secretin, somatostatin, thrombopoietin, thyroid-stimulating hormone (or thyrotropin), thyrotropin-releasing hormone, and vasoactive intestinal peptide, among others.

Other physiologically active peptides include tachykinin peptides, such as substance P, kassinin, neurokinin A, eledoisin, and neurokinin B; peptide PHI 27 (peptide histidine isoleucine 27); pancreatic polypeptide-related peptides, such as NPY (neuropeptide Y), PYY (peptide YY), and APP (avian pancreatic polypeptide); opioid peptides, such as proopiomelanocortin (POMC) peptides and prodynorphin peptides; AGG01; B-type natriuretic peptide (BNP); lactotripeptides; and peptides that inhibit PCSK9 (Zhang et al. 2014).

Exemplary antibodies include single-chain antibodies, single-domain antibodies (sdAbs), and single-chain variable fragments (scFvs).

Exemplary antimicrobial peptides include cathelicidins, defensins, protegrins, mastoparan, poneratoxin, cecropin, moricin, melittin, magainin, dermaseptin, nisin, and others. Other antimicrobial peptides include regIII-β and reg-III-γ, which are eukaryotic antimicrobial peptides produced in the intestine. Lactic acid bacteria are well known for their extensive heterogenic repertoire of antimicrobial compounds, including bacteriocins (Alvarez-Sieiro et al. 2016).

Other exemplary biologics include any of a number of antimicrobials. Lactic acid bacteria, for example, are well-known for their extensive heterogenic repertoire of antimicrobial compounds, including bacteriocins (Alvarez-Sieiro et al. 2016). Bacteriocins are small ribosomally-synthesized peptides that can inhibit or kill bacteria. The functional diversity of this family of antimicrobials is large, which is illustrated by the fact that bacteriocins can collectively target a wide-array of Gram-negative and Gram-positive bacteria (Cotter et al. 2013). Although narrow-spectrum bacteriocins may be preferential, the application of broad-spectrum bacteriocins may be useful to alleviate bacterial infections of unknown sources. Bacteriocin-mediated impact on the gut microbiota composition can be substantial. This was demonstrated for Abp118, a broad-spectrum bacteriocin produced by L. salivarius UCC118 (Riboulet-Bisson et al. 2012). See also Corr et al. 2007 (Corr S C, Li Y, Riedel C U, O'Toole P W, Hill C, Gahan C G. Bacteriocin production as a mechanism for the antiinfective activity of Lactobacillus salivarius UCC118. Proc Natl Acad Sci USA. 2007 May 1; 104(18):7617-21). By comparing the microbiota in mice and pigs between groups that were administrated with L. salivarius wild-type or L. salivariusΔabp118, it was confirmed that the presence of the bacteriocin-producing lactobacilli alters the gut microbiota composition without significance changes in microbial diversity. See also Kommineni et al. 2015. One example of a useful bacteriocin is nisin, which is produced by select Lactococcus lactis strains and streptococci. The 372 basepair gene encoding nisin (nisA) is one of the six natural nisin variants, and certain mutants NisA display enhanced activity against Gram-positive and Gram-negative pathogens (Field et al. 2008, Field et al. 2012).

Other exemplary biologics comprise lytic biologics. As used herein, “lytic biologic” refers to any biologic that causes or aids, either directly or indirectly, the lysis of a cell in which it is produced. Expression of a lytic biologic in a cell, for example, can induce lysis of the cell and any contents thereof, including any other biologics made by the cell.

Lytic biologics comprise lytic proteins. Lytic proteins are well known in the art. A number of lytic proteins, for example, are found in bacteriophages and serve to lyse cells during the lytic stages of the bacteriophage's life cycle. These include holins and lysins (Sheehan et al. 1999). During bacteriophage replication, biologically active lysins are present in the cytosol but require expression of a membrane protein, holin, to release the virions from the cell. When holin levels are optimal, the lysin can access the peptidoglycan layer for cleavage which leads to bacterial cell lysis (Wang et al. 2000). So far, five main groups of lysins have been identified that can be distinguished from one and another based on the cleavage specificity of the different bonds within the peptidoglycan (Fischetti 2009). Structurally, lysins can comprise a single catalytic domain, which generally is typical for lysins derived from bacteriophages targeting Gram-negative bacteria (Cheng et al. 1994). Bacteriophages targeting Gram-positive bacteria typically encode lysins that contain multiple domains: a N-terminal catalytic domain and a C-terminal cell-wall binding domain (Nelson et al. 2006, Navarre et al. 1999). A few lysins have been identified that have three domains (Becker et al. 2009).

A number of other lytic proteins are native to the cells themselves (Feliza et al. 2012, Jacobs et al. 1994, Jacobs et al. 1995, López et al. 1997). These lytic proteins can affect cell wall metabolism or introduce nicks in the cell wall. Five protein classes are differentiated by the wall component they attack (Loessner et al. 2005, Loessner et al. 2002).

In some versions, the biologic is a therapeutic biologic and a promoter operationally linked to the biologic coding sequence is a promoter inducible by an environmental condition of a disease that the therapeutic biologic is capable of treating.

An inducible promoter operably linked to a coding sequence of the biologic can be an inducible promoter sensitive to an environmental cue or condition, such as sugar concentration, bile acid concentration, or any other condition of the site in which expression of the coding sequence is desired.

In some versions, the biologic comprises a chimeric protein. A chimeric protein is a recombinant protein comprising sequences from two different native polypeptides. Any of the protein biologics described herein (or fragments thereof) can be fused with another polypeptide to generate a chimeric protein biologic.

In some versions, the biologic comprises a protein comprising an affinity tag. The affinity tags can be used for purification, detection with antibodies, or other uses. A number of affinity tags are known in the art. Exemplary affinity tags include the His tag, the Strep II tag, the T7 tag, the FLAG tag, the S tag, the HA tag, the c-Myc tag, the dihydrofolate reductase (DHFR) tag, the chitin binding domain tag, the calmodulin binding domain tag, the cellulose binding domain tag, and the HiBiT tag. The sequences of each of these tags are well-known in the art.

In some versions, the biologic is a fusion protein comprising a label. A label is a polypeptide sequence that is capable of being detected by any of a number methods. The label can be a fluorescent label (e.g., GFP, RFP, etc.), an enzymatic label (horseradish peroxidase (HRP), alkaline phosphatase (AP), glucose oxidase and β-galactosidase), an antibody, an antigen, or other types of polypeptide labels that can be fused to another polypeptide for detection.

The recombinant microorganisms of the invention can comprise bacteria. Bacteria of the invention can include certain commensal or probiotic bacteria, non-commensal bacteria, and other types of bacteria. The bacteria can include non-pathogenic, Gram-positive bacteria capable of anaerobic growth. The bacteria in some cases are viable in the gastrointestinal tract of mammals. The bacteria can be food grade. Other exemplary bacteria of the invention include E. coli.

Exemplary bacteria of the invention include species of lactic acid bacteria (i.e., species of the order Lactobacillales), such as those from the genera Lactobacillus, Limosilactobacillus, Leuconostoc, Pediococcus, Lactococcus, Streptococcus, Aerococcus, Carnobacterium, Enterococcus, Oenococcus, Fructobacillus, Sporolactobacillus, Tetragenococcus, Vagococcus, and Weissella.

Exemplary bacteria include species of the Lactobacillus genus. Exemplary species from the Lactobacillus genus include L. acetototerans, L. acidifarinae, L. acidipiscis, L. acidophilus, L. agilis, L. algidus, L. atimentarius, L. amytolyticus, L. amylophilus, L. amylotrophicus, L. amylovorus, L. animatis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. corynformis, L. crispatus, L. crustorum, L. curvatus, L. delbrueckii subsp. delbrueckii, L. delbrueckii subsp. butgaricus, L. delbrueckii subsp. lactis, L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. hammesii, L. hamsteri, L. harbinensis, L. hayakitensis, L. helveticus, L. hitgardii, L. homohiochii, L. iners, L. ingluviei, L. intestinalis, L. jensenii, L. johnsonii, L. katixensis, L. kefiranofaciens, L. kefiri, L. kimchii, L. kitasatonis, L. kunkeei, L. leichmannii, L. lindneri, L. malefermentans, L. mati, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracollinoides, L. parafarraginis, L. parakefiri, L. paratimentarius, L. paraplantarum, L. pentosus, L. perolens, L. plantarum, L. pontis, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae, and L. zymae.

Exemplary bacteria include species of the Limosilactobacillus genus. Exemplary species from the Limosilactobacillus genus include L. agrestis, L. albertensis, L. alvi, L. antri, L. balticus, L. caviae, L. coleohominis, L. equigenerosi, L. fastidiosus, L. fermentum, L. frumenti, L. gastricus, L. gorilla, L. ingluviei, L. mucosae, L. oris, L. panis, L. pontis, L. portuensis, L. reuteri, L. rudii, L. secaliphilus, L. urinaemulieris, and L. vaginalis.

Exemplary bacteria of the invention include species of Bifidobacterium. Exemplary species from the Bifidobacterium genus include B. actinocoloniiforme, B. adolescentis, B. aemilianum, B. aerophilum, B. aesculapii, B. amazonense, B. angulatum, B. animalis, B. anseris, B. apousia, B. apri, B. aquikefiri, B. asteroides, B. avesanii, B. biavatii, B. bifidum, B. bohemicum, B. bombi, B. boum, B. breve, B. callimiconis, B. callitrichidarum, B. callitrichos, B. canis, B. castoris, B. catenulatum, B. catulorum, B. cebidarum, B. choerinum, B. choladohabitans, B. choloepi, B. colobi, B. commune, B. criceti, B. crudilactis, B. cuniculi, B. dentium, B. dolichotidis, B. eriksonii, B. erythrocebi, B. eulemuris, B. faecale, B. felsineum, B. gallicum, B. gallinarum, B. globosum, B. goeldii, B. hapali, B. indicum, B. italicum, B. jacchi, B. lemurum, B. leontopitheci, B. longum, B. magnum, B. margollesii, B. merycicum, B. miconis, B. miconisargentati, B. minimum, B. mongoliense, B. moraviense, B. moukalabense, B. myosotis, B. oedipodis, B. olomucense, B. panos, B. parmae, B. platyrrhinorum, B. pluvialisilvae, B. polysaccharolyticum, B. pongonis, B. porcinum, B. primatium, B. pseudocatenulatum, B. pseudolongum, B. psychraerophilum, B. pullorum, B. ramosum, B. reuteri, B. rousetti, B. ruminale, B. ruminantium, B. saguini, B. saguinibicoloris, B. saimiriisciurei, B. samirii, B. santillanense, B. scaligerum, B. scardovii, B. simiarum, B. simiiventris, B. stellenboschense, B. subtile, B. thermacidophilum, B. thermophilum corrig., B. tibiigranuli, B. tissieri corrig., B. tsurumiense, B. urinalis, B. vansinderenii, B. vespertilionis, and B. xylocopae.

A bacterium used in the following examples is L. reuteri (Limosilactobacillus reuteri formerly referred to as Lactobacillus reuteri). In addition to L. reuteri, other particularly preferred bacteria include L. plantarum (e.g., L. plantarum BAA-793), L. rhamnosus (e.g., L. rhamnosus GG (L. rhamnosus ATCC 53103)), L. lactis (e.g., L. lactis MG1363), and L. casei.

The recombinant microorganisms of the invention can be administered to a subject.

The subject can include any animal, such as mammals or humans. The recombinant microorganisms can be administered orally, nasally, rectally, or via any other means of administration.

The expression systems of the invention can be used to introduce a biologic to a site. The site to which the biologic is introduced can be any site in which it is desired to introduce or produce the biologic. In some versions, the site is an in vitro site, for example, for producing the biologic for subsequent use (either in vitro or in vivo). Exemplary in vitro sites include test tubes, petri dishes, high-throughput device wells, bioreactors, etc.

In some versions, the site is an in vivo site. The in vivo site can be any site on or in a subject's body. The subject can be an animal, such as a mammal or a human.

In some versions, the site comprises a gastrointestinal tract of a subject. The methods of introducing the biologic to the site in such versions can comprise administering the recombinant microorganism to the gastrointestinal tract. Administering the recombinant microorganism to the gastrointestinal tract preferably occurs prior to the lysis of the recombinant microorganism. The recombinant microorganism can be administered to the gastrointestinal tract by any method known in the art. The recombinant microorganism can be administered orally, rectally, or directly into the gastrointestinal tract via a stoma. The recombinant microorganism is preferably administered directly into or upstream of the small intestines, so that the recombinant microorganism ultimately passes through or into the small intestines. The recombinant microorganism can be swallowed or introduced via a tube. The recombinant microorganism can be combined in a composition with a pharmaceutically acceptable excipient, carrier, buffer, stabilizer or other material well known to those skilled in the art. Such materials should be non-toxic and should not interfere with the efficacy of the recombinant microorganism. The precise nature of the carrier or other material may depend on the route of administration. The composition can be liquid, solid, or semi-solid. The composition can comprise a foodstuff or can take the form of a pharmaceutical composition. Those of relevant skill in the art are well able to prepare suitable compositions.

Expression of the biologic can be induced during, after, or prior to administering the recombinant microorganism to the site. Inducing expression of the biologic during administration to the site can be accomplished, for example, by co-administering an inducer with the recombinant microorganism in a single composition or simultaneously administering (whether in separate compositions or in a single composition) the inducer and the recombinant microorganism. In this manner, expression of the biologic can be initiated during the administration for subsequent introduction to the site.

Inducing expression of the biologic after administration to the site can be accomplished by administering an inducer after the recombinant microorganism is administered and, preferably, reaches the site. Depending on the type of recombinant microorganism and site, the recombinant microorganism can survive and/or proliferate at the site for a period until the inducer is administered. Administration of the inducer then induces expression of the biologic and introduction of the biologic to the site.

In some versions of the invention, one or more of the biologics can be introduced to a site without inducing lysis of the recombinant microorganism. In the case of polypeptide biologics, for example, the recombinant microorganism can comprise a recombinant gene configured to express and secrete the polypeptide. Elements for engineering a recombinant microorganism to secrete a polypeptide are well known in the art. Typical elements include a signal peptide-encoding sequence placed upstream of—and in-frame with—the coding sequence of the polypeptide to be secreted. The sequences of a large number of signal peptides for bacteria are known in the art. Exemplary signal peptide sequences are available on the world wide web at cbs.dtu.dk/services/SignalP/. The signal peptide can be cleaved from or remain intact on the polypeptide after secretion. The secreted polypeptide can be expressed from a coding sequence comprised within the regulatory sequence.

The recombinant microorganism of the invention can be engineered using any methods known in the art. General methods are provided in Green et al. 2012 (Green et al., Molecular Cloning: A Laboratory Manual, 4th ed., Cold Spring Harbor Laboratory Press, 2012). Methods for engineering lactic acid bacteria such as L. lactis are provided by van Pijkeren and Britton et al. 2012 (van Pijkeren J P, Britton R A. High efficiency recombineering in lactic acid bacteria. Nucleic Acids Res. 2012 May; 40(10):e76), van Pijkeren and Neoh et al. 2012 (van Pijkeren J-P, Neoh K M, Sirias D, Findley A S, Britton R A. 2012. Exploring optimization parameters to increase ssDNA recombineering in Lactococcus lactis and Lactobacillus reuteri. Bioengineered 3:209-217), Oh et al. 2014 (Oh J H, van Pijkeren J P. CRISPR-Cas9-assisted recombineering in Lactobacillus reuteri. Nucleic Acids Res. 2014; 42(17):e131), Barrangou et al. 2016 (Barrangou R, van Pijkeren J P. Exploiting CRISPR-Cas immune systems for genome editing in bacteria. Curr Opin Biotechnol. 2016 February; 37:61-8), and Zhang et al. 2018 (Zhang S, Oh J H, Alexander L M, Özçam M, van Pijkeren J P. d-Alanyl-d-Alanine Ligase as a Broad-Host-Range Counterselection Marker in Vancomycin-Resistant Lactic Acid Bacteria. J Bacteriol. 2018 Jun. 11; 200(13):e00607-17). The recombinant genes can be incorporated into the chromosome of the recombinant microorganism or can be included on an extra-chromosomal nucleic acid, such as a plasmid. The extra-chromosomal nucleic acid can replicate at any copy number in the recombinant microorganism and, accordingly, be a single-copy plasmid, a low-copy plasmid, or a high-copy plasmid. The extra-chromosomal nucleic acid is preferably substantially stable within the recombinant microorganism.

“Corresponding microorganism” refers to a microorganism of the same species having the same or substantially the same genetic and proteomic composition as a recombinant microorganism of the invention, with the exception of genetic and proteomic differences resulting from the modifications specified for the recombinant microorganisms of the invention. In some versions, the corresponding microorganism is the native version of the recombinant microorganism of the invention, i.e., the unmodified microorganism as found in nature. The terms “microorganism” and “microbe” are used interchangeably herein. “Corresponding native microorganism” refers to a native microorganism from which the recombinant microorganism is either directly or indirectly derived. The corresponding native microorganism will typically be a native microorganism having the closest genetic structure (e.g., highest percent genomic sequence identity) to the recombinant microorganism.

“Heterologous” as used herein refers to an element in an arrangement with another element that does not occur in nature. For example, a gene or protein that is heterologous to a given cell is a gene or protein that does not occur in the cell in nature. A promoter that is heterologous to a given coding sequence is a promoter that is not operably linked to the coding sequence in nature. A secretion signal sequence that is heterologous to a given protein (such as an enzyme) is a secretion signal sequence that is not operably linked with the protein in nature.

“Coding sequence” as used herein refers to a nucleic acid sequence in a gene that encodes a gene product. The term “coding sequence” encompasses sequences that include codons that are ultimately transcribed and translated into polypeptides as well as sequences that do not include codons and/or are merely transcribed (e.g., antisense RNA, etc.).

“Gene product” as used herein refers to any product resulting from expression (e.g., transcription or transcription and translation) of a gene. The term “gene product” explicitly encompasses polypeptides as well as nucleic acids such as RNA (e.g., mRNA, pri-microRNA, pre-microRNA, microRNA, antisense RNA (asRNA) etc.) and DNA (cDNA).

“Promoter” is used herein as understood in the art and typically refers to a nucleic acid sequence that confers transcription of an operably linked coding sequence. The promoters of the invention can comprise any promoter capable of being employed in the recombinant microorganisms of the invention. Promoters suitable for use in bacteria are typically derived from microbial or viral sources. Exemplary promoters include but are not limited to: promoters capable of recognizing the T4, T3, Sp6, and T7 polymerases; the P_{R} and P_{L} promoters of bacteriophage lambda; the trp, recA, heat shock, and lacZ promoters of E. coli; the alpha-amylase and the sigma-specific promoters of B. subtilis; the promoters of the bacteriophages of Bacillus; Streptomyces promoters; the int promoter of bacteriophage lambda; the bla promoter of the beta-lactamase gene of pBR322; and the CAT promoter of the chloramphenicol acetyl transferase gene. Prokaryotic promoters are reviewed by Glick, J. Ind. Microbiol. 1:277 (1987); Watson et al, Molecular Biology of the Gene, 4th Ed., Benjamin Cummins (1987); and Sambrook et al., In: Molecular Cloning: A Laboratory Manual, 3^{rd} ed., Cold Spring Harbor Laboratory Press (2001).

Any promoter of the invention (e.g., the repressor gene promoter, the antirepressor gene promoter, the biologic gene promoter, and any combination thereof) can be an inducible promoter. “Inducible promoter” as used herein refers to a regulated promoter that is active only in response to specific stimuli. Such specific stimuli are referred to herein as “inducers.” Exemplary inducers include proteins, metabolites, chemicals, and culture conditions. In some versions, the inducer is a particular concentration of a particular protein, metabolite, chemical, or culture condition. In some versions, the inducer is the presence of a particular protein, metabolite, chemical, or culture condition. In some versions, the inducer is the absence of a particular protein, metabolite, chemical, or culture condition. Exemplary inducible promoters include but are not limited to the lac promoter (regulated by IPTG or analogs thereof), the lacUV5 promoter (regulated by IPTG or analogs thereof), the tac promoter (regulated by IPTG or analogs thereof), the trc promoter (regulated by IPTG or analogs thereof), the araBAD promoter (regulated by L-arabinose), the phoA promoter (regulated by phosphate starvation), the recA promoter (regulated by nalidixic acid), the proU promoter (regulated by osmolarity changes), the cst-1 promoter (regulated by glucose starvation), the tetA promoter (regulated by tetracycline), the cadA promoter (regulated by pH), the nar promoter (regulated by anaerobic conditions), the p_{L} promoter (regulated by thermal shift), the cspA promoter (regulated by thermal shift), the T7 promoter (regulated by thermal shift), the T7-lac promoter (regulated by IPTG), the T3-lac promoter (regulated by IPTG), the T5-lac promoter (regulated by IPTG), the T4 gene 32 promoter (regulated by T4 infection), the nprM-lac promoter (regulated by IPTG), the VHb promoter (regulated by oxygen), the metallothionein promoter (regulated by heavy metals), the MMTV promoter (regulated by steroids such as dexamethasone) and variants thereof.

Any promoter of the invention (e.g., the repressor gene promoter, the antirepressor gene promoter, the biologic gene promoter, and any combination thereof) can be a constitutive promoter. “Constitutive promoter” as used herein refers to a promoter that is constitutively active, i.e., is not regulated by an inducer. Suitable constitutive promoters are known in the art and include constitutive adenovirus major late promoter, a constitutive MPSV promoter, and a constitutive CMV promoter.

“Operably linked” as used herein generally refers to a connection of two genetic elements in a manner wherein one can operate on or have effects on the other. “Operably linked” used in reference to a promoter and a coding sequence refers to a connection between the promoter and the coding sequence such that the coding sequence is under transcriptional control of the promoter. For example, promoters are generally positioned 5′ (upstream) of a coding sequence to be operably linked to the promoter. In the construction of heterologous promoter/coding sequence combinations, it is generally preferred to position the promoter at a distance from the transcription start site that is approximately the same as the distance between that promoter and the coding sequence it controls in its natural setting, i.e., in the gene from which the promoter is derived. As is known in the art, some variation in this distance can be accommodated without loss of promoter function.

“Overexpress” refers to the increased production of a gene product from a gene compared to an endogenous or basal product rate for that gene product. Methods of testing for overexpression are well known in the art, for example transcribed RNA levels can be assessed using RT-PCR and protein levels can be assessed using SDS-PAGE gel analysis.

“Introduce” as used herein with respect to an element such as a microorganism or a biologic, refers to any activity that results in the initial appearance or increased appearance of the element at a given site. Introducing a microorganism to a site can comprise, for example, inoculating, administering, culturing, and growing the microorganism at that site. Introducing a biologic to a site can comprise, for example, stimulating production of the biologic in the microorganism and/or releasing the biologic (e.g., through cell lysis or secretion) at the site.

The terms “identical,” “identity,” etc. in the context of two or more nucleic acid or polypeptide sequences, refer to two or more sequences or subsequences that are the same or have a specified percentage of amino acid residues or nucleotides that are the same, when compared and aligned for maximum correspondence, as measured using a sequence comparison algorithm available to a person of skill in the art.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)).

One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in identifying homologous sequences for use in the methods described herein.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Summary

Bacterial biotherapeutic delivery vehicles have the potential to treat a variety of diseases. This approach obviates the need to purify the recombinant effector molecule, allows delivery of therapeutics in situ via oral or intranasal administration, and protects the effector molecule during GI transit. Lactic acid bacteria have been broadly developed as therapeutic delivery vehicles, though risks associated with the colonization of a genetically modified microorganism have hitherto not been addressed. Without approaches to alleviate these risks, the application of genetically modified lactic acid bacteria in the clinical remains limited. Here, we present a novel biocontainment strategy that limits the ability of bacterial therapeutic delivery vehicles to adhere to human intestinal cells and mucus, using Limosilactobacillus reuteri as our model platform. We applied a dual-recombineering scheme to efficiently barcode and generate mutants in five previously characterized and four uncharacterized putative adhesins. We subsequently assayed the putative adhesins for their role in adhesion to human HT-29 cells and human enteroid monolayers. We combined the putative adhesin mutations into a single nonuple mutant, which was deficient in adhering to enteroid monolayers. These examples establish a novel biocontainment mechanism that lays a foundation for its application in other microbial therapeutic delivery candidates and furthers the progress of the L. reuteri delivery vehicle platform in human use.

### Introduction

We provide in the following examples a biocontainment strategy that prevents the adherence of biotherapeutic delivery vehicles by inactivating the proteins that allow bacteria to associate with host cells, thereby limiting their ability to persist or colonize the human gut. We chose L. reuteri ATCC 6475 to show this biocontainment strategy.

L. reuteri is a gut symbiont that has evolved to thrive in a large number of vertebrates (17-22), including humans (23). L. reuteri ATCC 6475 is an ideal choice to be developed as a therapeutic delivery vehicle due to its available genetic tools (24-26), its robustness during gastrointestinal transit, and its multiple probiotic (i.e. health-promoting) characteristics (27-30). L. reuteri reduces markers of metabolic syndrome in a diet-induced (high-sugar and high-fat) model of metabolic syndrome (31, 32), an effect that we improved by engineering L. reuteri 6475 to secrete murine interleukin-22 (IL-22) (32). We further enhanced L. reuteri 6475 by engineering a unique mechanism for phage-mediated lysis to improve the delivery of intracellularly accumulated therapeutics (33-35). L. reuteri encodes two prophages that are activated by molecules encountered during GI transit, such as short-chain fatty acids (SCFAs), leading to cell lysis (36). We recently demonstrated that phage-mediated lysis leading to the delivery of IL-22 by L. reuteri to alcohol binge-fed mice decreased indicators of liver disease (35) and increased the survival of mice exposed to total body irradiation (34). Now that we have clearly established the potential of L. reuteri as a therapeutic delivery vehicle, our goal is to develop and implement biocontainment strategies to bring L. reuteri a step closer to the clinic.

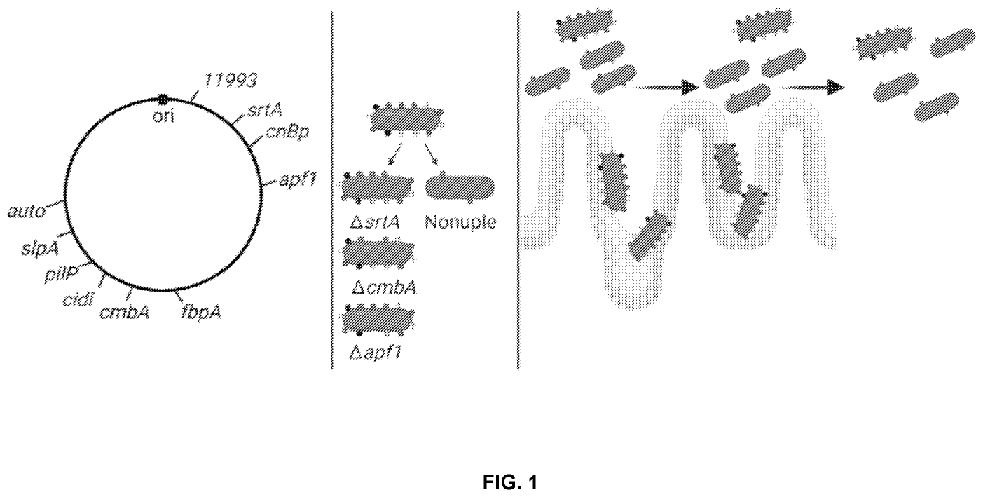

Gram-positive and probiotic bacteria like L. reuteri ATCC 6475 encode a variety of adhesin proteins that facilitate their association with host cells in the gut. Not only are these interactions important to colonize and persist, but bacterial adhesion also drives modulation of the host immune system (37-39). Sortase-dependent proteins (SDPs) are a particularly important group of proteins in both pathogenic and probiotic bacteria that facilitate adhesion and nutrient acquisition (40). SDPs are surface-associated proteins that are covalently coupled to the cell wall by the sortase enzyme (SrtA) (41). SDPs exhibit a conserved molecular structure that includes an N-terminal signal peptide that directs SDPs to surface localization (42), a C-terminal LPxTG motif that anchors SDPs to the cell wall (37, 40), a C-terminal transmembrane helix, and a positively charged tail (37, 40). We test in the present examples five L. reuteri SDPs (43). Other, non-SDP adhesins are classified in gram positives based on their specific interactions with host cells and extracellular components such as fibronectin-binding protein (FbpA) (44), collagen-binding protein (CnBp) (45, 46), and mucus-binding proteins (Mub and MapA) (47). Cell structures such as S-layer proteins (surface-layerproteins) (SlpA) (48), pili (PilP), flagella, and fimbriae can also interact with host cells and mucus (49). Lastly, proteins annotated as autolysins (50) and aggregation-promoting factors (apf) (51, 52) play a role in adhesion by facilitating interactions with collagen, fibronectin, and mucus (53). While L. reuteri 6475 lacks proteins and structures homologous to Mub, MapA, pili, and flagella, it does encode uncharacterized proteins homologous to SlpA, FbpA, autolysin, Apf, and CnBp. These adhesin homologs, along with SDPs, are ideal proteins to target for inactivation to hamper the bacterium's ability to colonize, persist, or act as an immunomodulator in the human gut. We therefore hypothesized that an L. reuteri strain lacking putative adhesins will have reduced potential to adhere to intestinal mucus and epithelial cells (FIG. 1).

In the present examples, we develop in L. reuteri a dual-recombineering method that introduces unique tags (barcodes) along with mutations to inactivate genes encoding putative adhesins. With this tool, we targeted ten putative adhesins of L. reuteri ATCC 6475 to evaluate their cumulative role in adhesion, including a previously characterized sortase, four previously identified SDPs (43), and five uncharacterized protein homologs that are not SDPs but have demonstrated roles in gram-positive adhesion to mucins and epithelial cells (44-46, 48, 50-52). We subsequently characterized the ecological role of each targeted adhesin. We describe the development and characterization of a single strain in which we inactivated nine genes (we excluded one mutation that conferred a growth defect) that putatively encode these adhesins. Functional characterization of this nonuple mutant revealed significantly reduced adhesive ability to human enteroid cells with no reduction in intestinal survival in mice, validating this approach as a biocontainment strategy. We expect that this novel biocontainment method can be applied to other bacteria engineered for therapeutic delivery, and further advances L. reuteri towards implementation in the clinic as a biotherapeutic delivery vehicle.

### Materials and Methods

### Bacterial Strains and Media.

The bacterial strains and plasmids used in the examples are listed in Table 1. Escherichia coli EC1000 and Lactococcus lactis MG1363 were used as intermediate cloning hosts. E. coli was cultured aerobically at 37° C. in lysogeny broth (LB; Teknova) and L. lactis was cultured statically at 30° C. in M17-broth (Difco; BD BioSciences) supplemented with 0.5% (w/v) glucose. Competent cells of E. coli EC1000 and L. lactis MG1363 were prepared as described previously (75, 76). Lactobacillus reuteri was grown in De Man, Rogosa, and Sharpe (MRS) medium (Difco, BD Biosciences) under hypoxic conditions (5% CO2, 2% O2) at 37° C. L. reuteri competent cells were prepared as described previously (25). As needed, erythromycin was supplemented at 5 μg/ml for the L. reuteri strains and 300 μg/ml for E. coli EC1000. Chloramphenicol was added as needed at 5 μg/ml for L. reuteri and L. lactis. Tetracycline was added as needed at 25 μg/ml for L. reuteri and 10 μg/ml for L. lactis.

TABLE 1
Bacterial strains and plasmids used in this study.

| | Characteristics^{†} | Source/Ref .* |
|---|---|---|
| Strains | | |
| (Name/VPL) | | |
| E. coli | Derivative of E. coli MC1000 in which repA is integrated in | (Leenhouts et |
| EC1000 | chromosome | al, 1996) |
| L. lactis | Plasmid-free derivative of L. lactis subsp. cremoris | (Wegmann et |
| MG1363 | NCDO712 | al, 2007) |
| L. reuteri | Human breast milk isolate | Biogaia A.B. |
| ATCC PTA | | |
| 6475 | | |
| VPL3187 | Mutant harboring pVPL3004 and pVPL3016 | (Oh and van |
| | | Pijkeren, 2014) |
| VPL4011 | Mutant with inactivated cat (cat*, L141*) gene insertion | This work |
| VPL4018 | ΔsrtA::oVPL449 (K150*V151Q) | (Oh and van |
| | | Pijkeren, 2014) |
| VPL4052 | Mutant with cat* restored to functional cat with oVPL428 | This work |
| VPL4359 | Derivative of VPL4011; ΔcmbA::oVPL3796 (P282*N283D); | This work |
| | cat::oVPL3848 | |
| VPL4360 | Derivative of VPL4011; Δcyclic-phosphodiesterase | This work |
| | (Δcidi)::oVPL3802 (P58*Q59*); cat::oVPL3848 | |
| VPL4361 | Derivative of VPL4011; ΔpilP::oVPL3808 (N162*Q163*); | This work |
| | cat::oVPL3848 | |
| VPL4362 | Derivative of VPL4011; ΔslpA::oVPL3814 (V102*Q103*); | This work |
| | cat::oVPL3848 | |
| VPL4363 | Derivative of VPL4011; ΔsrtA::oVPL449 (K150*V151Q); | This work |
| | cat::oVPL3848 | |
| VPL4364 | Derivative of VPL4011; ΔfbpA::oVPL3763 (N68*P69*); | This work |
| | cat::oVPL3848 | |
| VPL4365 | Derivative of VPL4011; Δapf1::oVPL3850 | This work |
| | (D82EG83*E84*); cat::oVPL3848 | |
| VPL4367 | Derivative of VPL4011; Δautolysin (Δauto)::oVPL3856 | This work |
| | (P99*K100*); cat::oVPL3848 | |
| VPL4368 | Derivative of VPL4011; ΔLAR_0044 (Δ11993)::oVPL3694 | This work |
| | (Q211RG212*); cat::oVPL3848 | |
| VPL4366 | Mutant with 9 inactivated putative adhesion genes; nonuple | This work |
| VPL4379 | Derivative of L. reuteri VPL1014; ΔcnBp::oVPL3939 | This work |
| | (M76RG77*) | |
| Plasmids | | |
| pJP042 | ssDNA recombineering plasmid, Em^{R} derivative of pSIP411 | (van Pijkeren |
| | in which | and Britton, |
| | the gusA gene is replaced with recT1 derived from L. reuteri | 2012) |
| | ATCC PTA | |
| | 6475. recT1 is under the control of an inducible promoter | |
| pVPL3583 | pJP028 vector | (Alexander et |
| | | al 2019) |
| pVPL3002 | pORI19 harboring L. reuteri derived ddlF258Y | (Zhang et al |
| | | 2018) |
| pVPL3004 | Em^{R}, derivative of pNZ9530 in which nisR and nisK genes | (Oh and van |
| | were replaced | Pijkeren, 2014) |
| | with the tracrRNA, cas9 and CRISPR array derived from | |
| | pCAS9 | |
| pVPL3017 | ssDNA recombineering plasmid, Cm^{R} derivative of pJP042 | (Lab stock) |
| pVPL3115 | Derivative of pNZ8048 harboring the CRISPR array | (Oh and van |
| | | Pijkeren, 2014) |
| pVPL3031 | Derivative of pNZ8048 harboring cat* and ery. | This work |
| | pVPL3002 derivative. Suicide shuttle vector with flanking | |
| pVPL3038 | sequence of a non-coding region in L. reuteri designed for | (Oh et al 2020) |
| | chromosomal insertions | |
| pVPL3047 | pVPL3038 derivative with inactivated cat* integration | This work |
| | cassette | |
| pVPL31134 | pJP028 derivative, pCtl-ThyA | (Alexander et |
| | | al 2019) |
| pVPL31464 | pJP028 derivative, pIL-22-ThyA | (Zhang et al |
| | | 2020) |
| pVPL31467 | pJP028 derivative; cmbA complementation plasmid | This work |
| pVPL31514 | pJP028 derivative, pilP complementation plasmid lacking cat | This work |
| | gene | |
| pVPL31515 | pJP028 derivative, slpA complementation plasmid lacking cat | This work |
| | gene | |
| pVPL31516 | pJP028 derivative, srtA complementation plasmid lacking cat | This work |
| | gene | |
| pVPL31517 | pJP028 derivative, fpbA complementation plasmid lacking cat | This work |
| | gene | |
| pVPL31518 | pJP028 derivative, autolysin (auto) complementation plasmid | This work |
| | lacking cat gene | |
| pVPL31519 | pJP028 derivative, apf1 complementation plasmid lacking cat | This work |
| | gene | |
| pVPL31520 | pJP028 derivative, 11993 complementation plasmid lacking | This work |
| | cat gene | |
| pVPL31521 | pVPL31467 derivative lacking cat gene | This work |
| pVPL31522 | pJP028 derivative, cyclic-phosphodiesterase (cidi) | This work |
| | complementation plasmid lacking cat gene | |

VPL: Van Pijkeren Lab strain identification number. pVPL: Van Pijkeren Lab plasmid identification number.
^{†}repA: replication initiation protein; Em^{R}: erythromycin-resistant; Cm^{R}: chloramphenicol-resistant;
*nonsense mutation; cat: chloramphenicol acetyltransferase; ery: 23S ribosomal RNA methyltransferase; LAR_#### refer to closed reference genome Limosilactobacillus reuteri JCM1112.
ddlA: d-alanine-d-alanine ligase (LAR_1277). The locus tags for putative adhesion mutants can be found in Table 3.

### Cell and Organoid Culture.

The human colorectal adenocarcinoma cell line HT-29 (ATCC HTB-38) was obtained from the American Type Culture Collection. HT-29 cells were grown in Dulbecco's modified Eagle's medium containing 4.5 g/L glucose with L-glutamine and sodium pyruvate (VWR, 45000-304) supplemented with 10% (v/v) heat-inactivated fetal bovine serum (Sigma-Aldrich, 12306C-500ML). Cells were maintained at 37° C. in a humidified atmosphere of 5% CO2 and split when the cells reached 70-90% confluence. Human colon cancer enteroids (121 CRC) were isolated and cultured as described previously (77). Briefly, human tissue from needle biopsy or surgical resection was placed in chelation buffer and then digested in stock media: advanced DMEM/F12 medium (Invitrogen, Carlsbad, CA) containing 10% fetal bovine serum, collagenase (1 mg/ml), dispase (12.5 μg/ml) and 1% (v/v) penicillin and streptomycin. The tissues were disrupted with intermittent shaking. The cell suspension was then separated from digestion buffer at 300×g for five minutes at 4° C. and washed once with 1×PBS. PBS was removed from the cell pellet and the pellet was resuspended in DMEM/F12 containing 1× glutamax, 10 mM HEPES, and 1% (v/v) penicillin and streptomycin. Cell suspensions were maintained on ice and mixed with Matrigel at a 1:1 ratio before being plated as droplets onto 24-well culture plates and incubated at 37° C. Plates were inverted after two to three minutes of incubation. After the mixture had solidified, cultures were overlaid with feeding medium consisting of 50% (v/v) stock media and 50% (v/v) conditioned medium obtained from WNT3a L cell line (ATCC CRL-2647) mixed with EGF (50 ng/ml). To maintain enteroids, media was changed every other day. To generate 121 CRC organoid monolayers, enteroids were disrupted and sheared using a 27-G needle before centrifugation at 300×g at 4° C. for five minutes. Matrigel was aspirated from the cells, and the cells were resuspended in ice-cold PBS before centrifugation at 300×g at 4° C. for five minutes. Trypsin (0.25% w/v) was then added to the organoid pellet and incubated at 37° C. for 10 minutes. The cell suspension was centrifuged again, and the supernatant was removed before resuspending the cells in feeding medium and adding them to a 24-well plate coated with 0.5% (v/v) collagen I. Monolayer medium was changed every two days until confluence was reached. All concentrations listed are final concentrations.

### Imaging and Mucin Staining of Human 121 C Enteroids.

Enteroids grown on glass coverslips were fixed in 60% anhydrous methanol, 30% chloroform, and 10% glacial acetic acid in IX phosphate-buffered saline (PBS) for 45 minutes at room temperature (RT), then washed twice with 0.2% (v/v) Triton X-100 in PBS. Fixed enteroids were permeabilized and blocked by incubating for 60 minutes in PBS with 3% (w/v) bovine serum albumin (BSA), 0.2% (v/v) Triton X-100 at RT. Enteroids were stained with 40,6-Diamidino-2-phenylindole (DAPI) and Mucin 2 (Muc2) antibody (F-2) Alexa Fluor 488. Muc2 antibody was diluted 1:1000 in 3% (v/v) BSA and 0.2% (v/v) Triton X-100 in PBS, added to cells, and incubated for 60 minutes at RT. Cells were net washed three times for five minutes each at RT with 0.2% (v/v) Triton X-100 in PBS. Secondary antibody was then added at a 1:1000 dilution in 3% (v/) BSA and 0.2% (v/v) Triton X-100 in PBS and incubated 45 minutes at RT. Cells were again washed three times for five minutes each at RT with 0.2% (v/v) Triton X-100 in PBS. After one final wash with PBS, cells were dried before adding Vectashield (˜10 μL), covered with a glass slip and imaged on Zeiss Axioplan III equipped with a triple-pass (DAPI/fluorescein isothiocyanate [FITC]/Texas Red) emission cube, differential interference contrast optics, and a nonochromatic Axiocam camera operated by Zen software (Zeiss) and processed using GIMP 2 software.

### Bioinformatic Analyses.

We included several sortase-dependent proteins (SDPs) in our experiments (43, 78). Genes previously identified as pseudogenes were excluded from further analysis (43). Homologs of previously characterized adhesin proteins were used as a query to search the L. reuteri JCM1112 chromosome using BLASTP at the National Center for Biotechnology Information website (http://www.ncbi.nlm.nih.gov) (see Table 2 for accession numbers used as queries and references). Adhesin homologs were also analyzed for SDP characteristics. The sorting motif LPxTG was manually searched for in the protein sequences. YSIRK-G/S signal sequences (pfam04650), cell wall anchor domains (TIGR01167) and other protein domains were searched for in InterPro (79). Secretion signal peptides were predicted with SignalP 5.0 (80) and transmembrane helices were predicted with TMHMM 2.0 (http://www.cbs.dtu.dklservices/TMHMM). Repeats in the protein sequences were identified using RADAR (world wide web at ebi.ac.uk/Tools/pfa/radar).

TABLE 2
In silico analysis of putative adhesion proteins in L. reuteri.

| | | | Predicted SP | | | | | |
|---|---|---|---|---|---|---|---|---|
| | | | (Y/N) and | | | | | |
| | | | YSIRK | | Sortase | Repeat | | |
| Gene | | | Cleavage Site^{c} | Hydrophobic | dependent? | region | | |
| target | Locus^{a} | Rationale^{b} | [Y/N] | CTD^{d} region | (Y/N) | (Y/N) | Characteristics | Reference |
| srtA | LAR_0227 | Sortase | — | — | — | N | Inactivation of srtA reduced | Jensen et al. |
| | | | | | | | adhesion to Caco-2 | 2014 |
| | | | | | | | cells by L. reuteri 6475 | |
| cmbA | LAR_0958 | SDP | Y[Y] | Y | Y | Y | Inactivation of cmbA reduced | Jensen et al. |
| | | | | | | | adhesion to Caco-2 | 2014 |
| | | | | | | | cells by L. reuteri 6475 | |
| 11993 | LAR_0044 | SDP | Y[N] | Y | Y | Y | Inactivation did not result in | Jensen et al. |
| | | | | | | | adherence defect to Caco-2 | 2014 |
| | | | | | | | cells by L. reuteri | |
| ci-phospho- | LAR_0983 | SDP | Y[N] | Y | Y | Y | Inactivation did not result in | Jensen et al. |
| diesterase | | | | | | | adherence defect to Caco-2 | 2014 |
| | | | | | | | cells by L. reuteri | |
| LAR_0903 | LAR_0903 | SDP | Y[N] | Y | Y | Y | WP_096039546, | Jensen et al. |
| | | | | | | | L. raffinolactis = 28% | 2014, |
| | | | | | | | protein sequence | Mulligan |
| | | | | | | | identity; secondary | and Snell, |
| | | | | | | | start-site indicates | 1977, and |
| | | | | | | | LAR_0903 is an SDP | Ito et al. |
| | | | | | | | | 2019 |
| pilP | LAR_0989 | SDP | Y[N] | Y | Y | Y | PilP and Rib regions; | Jensen et al. |
| | | | | | | | Inactivation did not result in | 2014 |
| | | | | | | | adherence defect to Caco-2 cells | |
| | | | | | | | by L. reuteri | |
| fbpA | LAR_0878 | Fibronectin- | N[n/a] | N | N | Y | FbpA pfam entry PF05833 | Muñoz- |
| | | binding | | | | | | Provencio |
| | | protein | | | | | | et al. 2010 |
| autolysin | LAR_1284 | Autolysin | Y[N] | N | N | Y | NP_466081.1, L. monocytogenes | Milohanic |
| | | | | | | | autolysin = 50% protein | et al. 2001 |
| | | | | | | | sequence identity; | |
| | | | | | | | evidence of role in adhesion in | |
| | | | | | | | S. aureus, L. monocytogenes | |
| | | | | | | | and L. acidophilus | |
| slpA | LAR_1193 | Surface-layer | Y[Y] | Y | N | Y | AJP46713.1, L. acidophilus | Sahay et al. |
| | | protein | | | | | surface-layer protein = | 2016 and |
| | | | | | | | 62.69% protein | Buck et al. |
| | | | | | | | sequence identity | 2005 |
| apf1 | LAR_0410 | APF | Y[N] | Y | N | Y | AAO86515.1, L. gasseri | Ventura et |
| | | | | | | | Apf1 = 85% protein | al. 2002 |
| | | | | | | | sequence identity | and Hevia |
| | | | | | | | | et al. 2013 |
| cnbP | LAR_0284 | Collagen- | Y[N] | Y | N | Y | ADN22849.1, L. reuteri Pg4 | Hsueh et al. |
| | | binding | | | | | collagen-binding protein = 98% | 2010 and |
| | | protein | | | | | protein sequence identity; | Miyoshi et |
| | | | | | | | periplasmic binding region | al. 2006 |

^{a}Loci are based on the L. reuteri reference genome L. reuteri JCM1112, and can be accessed at http://www.ncbi.nlm.nih.gov.
^{b}SDP, sortase-dependent protein; SDPs were indicated by the presence of: LPxTG motif identified through manual search, YSIRK-G/S (pfam04650) [Y] or non-YSIRK signal sequence [N]), and cell wall anchor domains (TIFR01167).
^{c}SP, signal peptide; presence of signal peptide and cleavage site were determined by SignalP-5.0 (Armenteros et al 2019). Protein motifs and domains were identified by Interpro 83.0 and searched for with BLASTP at the National Center for Biotechnology Information website (http://www.ncbi.nlm.nih.gov) (Blum et al 2020).
^{d}CTD, C-terminal domain.

Generation of In-Frame Stop Codon in chloramphenicol acetyltransferase.

Plasmid pJP028 harbors cat and ery, which encode chloramphenicol and erythromycin resistance, respectively. Expression of cat is placed under the control of the PHELP-promoter (81). Oligonucleotides oVPL261-262 (Table 3) are complementary to the cat gene with exception of three bases; subsequent amplification and self-ligation of the amplicon yielded pVPL3031 which contains an in-frame stop codon in cat (cat*; L141*).

TABLE 3
Oligonucleotides used for plasmid construction in this study.

| Oligo | | |
|---|---|---|
| Name | Sequence (5′-3′)^{†} | Target/comment^{#} |
| oVPL202 | Atgaactttaataaaattgatttagac | Fwd, cat gene from pVPL3031 |
| | (SEQ ID NO: 23) | |
| oVPL203 | ttataaaagccagtcattaggcc (SEQ ID | Rev, cat gene from pVPL3031 |
| | NO: 24) | |
| oVPL261 | TCAagaaaaagcattttcaggtatagg | Fwd, introduces internal stop codon into cat |
| | (SEQ ID NO: 25) | from pVPL3031, generating cat*(L141*) |
| oVPL262 | tctattattccttggacttcattt (SEQ ID | Rev, introduces internal stop codon into cat |
| | NO: 26) | from pVPL3031, generating cat*(L141*) |
| oVPL271 | ttaaaaattaatctttccagtaataatcaaca | Fwd, internal oligonucleotide for pVPL3038 |
| | tc (SEQ ID NO: 27) | backbone for cloning |
| | | cat* gene |
| oVPL272 | ttaaaatgtaggtttaatttttagggc | Rev, internal oligonucleotide for pVPL3038 |
| | (SEQ ID NO: 28) | backbone for cloning |
| | | cat* gene |
| oVPL283 | aagcagtcaaaaagccctaaaaattaaac | Fwd, oligonucleotide with 40 bp clamps to |
| | ctacattttaacattatgctttggcagtttattc | amplicon 265-266 to clone cat* gene (lagging |
| | ttgacatg (SEQ ID NO: 29) | strand orientation) via Gibson assembly |
| oVPL284 | tgcgctgatgttgattattactggaaagatta | Rev, oligonucleotide with 40 bp clamps to |
| | atttttaatttgattgatagccaaaaagcagc | amplicon 265-266 to clone cat* gene (lagging |
| | ag (SEQ ID NO: 30) | strand orientation) via Gibson assembly |
| oVPL309 | tctcgctttgattgttctatcgaaag (SEQ | Rev, for amplifying pJP028 backbone omitting |
| | ID NO: 31) | cat gene |
| oVPL310 | ataaggaagataaatcccataagggc | Fwd, for amplifying pJP028 backbone |
| | (SEQ ID NO:32) | omitting cat gene |
| oVPL334 | aactttcgccattaatgtgttttatcgg | Fwd, for single-crossover and double- |
| | (SEQ ID NO: 33) | crossover screening of cat* |
| | | insertion |
| oVPL335 | agacagatgacaagccctttagc (SEQ | Rev, for single-crossover and double- |
| | ID NO: 34) | crossover screening of cat* |
| | | Insertion |
| oVPL363 | taatatgagataatgccgactgtac (SEQ | Fwd, for screening for presence of pThyA-Ctl |
| | ID NO: 35) | and pIL-22-ThyA plasmids |
| oVPL728 | ttcattacatccatgggtgtc (SEQ ID | Rev, for screening for presence of pThyA-Ctl |
| | NO: 36) | and pIL-22-ThyA plasmids |
| oVPL736 | tgaatgagtgagtcaacttg (SEQ ID | Fwd, for amplifying pMutL promoter |
| | NO: 37) | sequence from pSIP411:pMutL-ThyA |
| oVPL737 | taaatatcaccttatttcaa (SEQ ID | Rev, for amplifying pMutL promoter sequence |
| | NO: 38) | from pSIP411:pMutL-ThyA |
| oVPL1286 | tgatctttgaaccaaaattag (SEQ ID | Fwd, for amplifying pJP028 backbone |
| | NO: 39) | |
| oVPL1408 | agaaaaccgactgtaaaaagtacag | Rev, for amplifying pJP028 backbone |
| | (SEQ ID NO: 40) | |
| oVPL4033 | atgctatcaagaaaaaattataagga | Fwd, for amplifying cmbA (LAR_0958) gene |
| | (SEQ ID NO: 41) | |
| oVPL4034 | ctaatcatgtttacgcttcttgcc (SEQ | Rev, for amplifying cmbA (LAR_0958) gene |
| | ID NO: 42) | |
| oVPL4035 | gcagcagaaattgaaataaggtgatattta | LCR bridging oligonucleotide for ligating |
| | atgctatcaagaaaaaattataaggaaact | pMutL promoter sequence to cmbA for |
| | (SEQ ID NO: 43) | insertion into pJP028 for complementation |
| oVPL4036 | gccgactgtactttttacagtcggttttcttg | LCR bridging oligonucleotide for insertion of |
| | aatgagtgagtcaacttgaattatttgc | pMutL into pJP028 for adhesion protein |
| | (SEQ ID NO: 44) | complementation |
| oVPL4037 | ggtttgggcaagaagcgtaaacatgatta | LCR bridging oligonucleotide for insertion of |
| | gtgatctttgaaccaaaattagaaaaccaa | cmbA into pJP028 for complementation |
| | g (SEQ ID NO: 45) | |
| oVPL4038 | gtgaaaaaagataaaaagcga (SEQ ID | Fwd, for amplifying srtA (LAR_0227) gene |
| | NO: 46) | |
| oVPL4039 | ttaacgacctgtcgtatatt (SEQ ID | Rev, for amplifying srtA (LAR_0227) gene |
| | NO: 47) | |
| oVPL4040 | gcagcagaaattgaaataaggtgatattta | LCR bridging oligonucleotide for ligating |
| | gtgaaaaaagataaaaagcgatcatttgaa | pMutL promoter sequence to srtA for insertion |
| | (SEQ ID NO: 48) | into pJP028 for complementation |
| oVPL4041 | tttagcgaaaaatatacgacaggtcgttaat | LCR bridging oligonucleotide for insertion of |
| | gatctttgaaccaaaattagaaaaccaag | srtA into pJP028 for complementation |
| | (SEQ ID NO: 49) | |
| oVPL4045 | atgtcttttgacggcttg (SEQ ID | Fwd, for amplifying fbpA (LAR_0878) gene |
| | NO: 50) | |
| oVPL4046 | ttagttagaaagtttatgcggtgt (SEQ | Rev, for amplifying fbpA (LAR_0878) gene |
| | ID NO: 51) | |
| oVPL4047 | tgcatgagtaaacaagccgtcaaaagaca | LCR bridging oligonucleotide for ligating |
| | ttaaatatcaccttatttcaatttctgctgc | pMutL promoter sequence to fbpA for insertion |
| | (SEQ ID NO: 52) | into pJP028 for complementation |
| oVPL4048 | tatgtaacaccgcataaactttctaactaat | LCR bridging oligonucleotide for insertion of |
| | gatctttgaaccaaaattagaaaaccaag | fbpA into pJP028 for complementation |
| | (SEQ ID NO: 53) | |
| oVPL4049 | atgaagaataatagttcaaaatattg | Fwd, for amplifying cyclic-phosphodiesterase |
| | (SEQ ID NO: 54) | (cidi) (LAR_0983) gene |
| oVPL4050 | ttaagcatgtttacgctt (SEQ ID | Rev, for amplifying cyclic-phosphodiesterase |
| | NO: 55) | (cidi) (LAR_0983) gene |
| oVPL4051 | gcagcagaaattgaaataaggtgatattta | LCR bridging oligonucleotide for ligating |
| | atgaagaataatagttcaaaatattgttta | pMutL promoter sequence to cidi for insertion |
| | (SEQ ID NO: 56) | into pJP028 for complementation |
| oVPL4052 | attattgatcgcaagcgtaaacatgcttaat | LCR bridging oligonucleotide for insertion of |
| | gatctttgaaccaaaattagaaaaccaag | cidi into pJP028 for complementation |
| | (SEQ ID NO: 57) | |
| oVPL4053 | atgaagaaaagaaaatta (SEQ ID | Fwd, for amplifying pilP (LAR_0989) gene |
| | NO: 58) | |
| oVPL4054 | ttattcgtaccgtttaa (SEQ ID | Rev, for amplifying pilP (LAR_0989) gene |
| | NO: 59) | |
| oVPL4055 | gcagcagaaattgaaataaggtgatattta | LCR bridging oligonucleotide for ligating |
| | atgaagaaaagaaaattaaagaagagttta | pMutL promoter sequence to pilP for insertion |
| | (SEQ ID NO: 60) | into pJP028 for complementation |
| oVPL4056 | attggggcaacacttaaacggtacgaata | LCR bridging oligonucleotide for insertion of |
| | atgatctttgaaccaaaattagaaaaccaa | pilP into pJP028 for complementation |
| | g (SEQ ID NO: 61) | |
| oVPL4057 | atgtcgaagaacaatgcac (SEQ ID | Fwd, for amplifying slpA (LAR_1193) gene |
| | NO: 62) | |
| oVPL4058 | tcagtaatagttgggtttatctgt | Rev, for amplifying slpA (LAR_1193) gene |
| | (SEQ ID NO: 63) | |
| oVPL4059 | gcagcagaaattgaaataaggtgatattta | LCR bridging oligonucleotide for ligating |
| | atgtcgaagaacaatgcacaagaatatgta | pMutL promoter sequence to slpA for insertion |
| | (SEQ ID NO: 64) | into pJP028 for complementation |
| oVPL4060 | gggatgacagataaacccaactattactga | LCR bridging oligonucleotide for insertion of |
| | tgatctttgaaccaaaattagaaaaccaag | slpA into pJP028 for complementation |
| | (SEQ ID NO: 65) | |
| oVPL4061 | gtgactaataaaaagcatta (SEQ ID | Fwd, for amplifying autolysin, (auto) |
| | NO: 66) | (LAR_1284) gene |
| oVPL4062 | ttagaattcaccataatat (SEQ ID | Rev, for amplifying autolysin, (auto) |
| | NO: 67) | (LAR_1284) gene |
| oVPL4063 | gcagcagaaattgaaataaggtgatattta | LCR bridging oligonucleotide for ligating |
| | gtgactaataaaaagcattataaattatat | pMutL promoter sequence to autolysin for |
| | (SEQ ID NO: 68) | insertion into pJP028 for complementation |
| oVPL4064 | ttggtaagcctatattatggtgaattctaatg | LCR bridging oligonucleotide for insertion of |
| | atctttgaaccaaaattagaaaaccaag | autolysin into pJP028 for complementation |
| | (SEQ ID NO: 69) | |
| oVPL4065 | atgatttctaagaaaaactttg (SEQ | Fwd, for amplifying apf1 (LAR_0410) gene |
| | ID NO: 70) | |
| oVPL4066 | ttagtaccagccattagct (SEQ ID | Rev, for amplifying apf1 (LAR_0410) gene |
| | NO: 71) | |
| oVPL4067 | gcagcagaaattgaaataaggtgatattta | LCR bridging oligonucleotide for ligating |
| | atgatttctaagaaaaactttgctaaagta | pMutL promoter sequence to apf1 for insertion |
| | (SEQ ID NO: 72) | into pJP028 for complementation |
| oVPL4068 | gctcactggcaagctaatggctggtactaa | LCR bridging oligonucleotide for insertion of |
| | tgatctttgaaccaaaattagaaaaccaag | apf1 into pJP028 for complementation |
| | (SEQ ID NO: 73) | |
| oVPL4069 | atgagaaattcgaatacaaataattg | Fwd, for amplifying 11993 (LAR_0044) gene |
| | (SEQ ID NO: 74) | |
| oVPL4070 | ttagttgtggcgcttctttg (SEQ ID | Rev, for amplifying 11993 (LAR_0044) gene |
| | NO: 75) | |
| oVPL4071 | gcagcagaaattgaaataaggtgatattta | LCR bridging oligonucleotide for ligating |
| | atgagaaattcgaatacaaataattggcgt | pMutL promoter sequence to 11993 for |
| | (SEQ ID NO: 76) | insertion into pJP028 for complementation |
| oVPL4072 | acttacagctcaaagaagcgccacaacta | LCR bridging oligonucleotide for insertion of |
| | atgatctttgaaccaaaattagaaaaccaa | 11993 into pJP028 for complementation |
| | g (SEQ ID NO: 77) | |

oVPL: Van Pijkeren Lab oligonucleotide identification number. Uppercase bases indicate mismatches with wild-type sequence.
^{#}: Fwd: forward; Rev: reverse; oligo: oligonucleotide. cat: chloramphenicol acetyltransferase;
LAR_#### refer to closed reference genome Limosilactobacillus reuteri JCM1112.
^{†}: * indicates nonsense mutation.

Integration of Cat* in L. reuteri Chromosome.

The oligonucleotides used in the present examples can be found in Table 3. The PHELP::cat* cassette was amplified from pVPL3031 with oVPL283-284 and pVPL3038 (82) was amplified with oVPL271-272, followed by Gibson assembly (83) to yield pVPL3047. L. reuteri was transformed with 5 μg pVPL3047, and we screened by PCR for upstream and downstream single-crossover homologous recombination with oligonucleotides oVPL203-334-335 and oVPL202-334-335, respectively. Integration of cat* following double-crossover recombination was confirmed with oligonucleotide pair oVPL334-335 to yield L. reuteri VPL4011. Integration of oVPL283 by ssDNA recombineering (24) reverted the in-frame stop codon to its original DNA sequence to yield L. reuteri VPL4052, which served as the control for growth and adhesion experiments.

Construction of L. reuteri Putative Adhesion Protein Mutants and Barcoding.

Putative adhesin mutants were generated by single-stranded DNA (ssDNA) recombineering as described previously (24). Briefly, VPL4011 harboring pVPL2032, which provides inducible expression of the phage recombinase RecT, was simultaneously transformed with 100 μg oVPL3848, a degenerative oligonucleotide, targeting cat* and 100 μg oligonucleotide targeting a putative adhesin (Table 4). To restore cat, oVPL3848 contains three adjacent randomized bases targeting the stop codon and three additional randomized bases targeting nearby wobble bases in codons encoding A138, S140, and S142. Incorporation of oVPL3848 in the chromosome modifies the stop-codon and wobble bases creating a mixture of unique barcodes. Following selection on MRS supplemented with chloramphenicol, recombinant genotypes of genes encoding putative adhesins were identified by a mismatch amplification mutation assay (MAMA) PCR (84, 85). The integrity of recombinant genotypes was confirmed by Sanger Sequencing. Each barcode was subsequently introduced in VPL4011 harboring pVPL2032 via recombineering, resulting in a group of control strains (Table 4).

TABLE 4
Recombineering and mutant screening oligonucleotides.

| Oligo name | sequence (5′-3′) | Target/comment^{#} | Locus* | Mutation(s)^{†} |
|---|---|---|---|---|
| oVPL236 | tcaaaccaccaggaccaagcgctgaa | Targets rpoB | LAR_1402 | H488R |
| | agacgacgcttTCTGCttaattcac | | | |
| | ctaatgggttggtttgatccatgaactgg | | | |
| | (SEQ ID NO: 78) | | | |
| oVPL449 | aaacgcgatccatgttggtgatataaatc | Targets srtA | LAR_0227 | K150*V151Q |
| | atctgccctTGTCAagcatgatagt | | | |
| | acaatggagaaaagaggattttgctcc | | | |
| | (SEQ ID NO: 79) | | | |
| oVPL468 | tcctaattcgcaaaataagcagagg | Fwd, starts 500 bp upstream | | |
| | (SEQ ID NO: 80) | of site mutated by oVPL449 | | |
| oVPL469 | aatggattacaaatacaggcaaaatcc | Rev, starts 500 bp | | |
| | (SEQ ID NO: 81) | downstream of site mutated | | |
| | | byoVPL449 | | |
| oVPL470 | ttggtgatataaatcatctgccctTGT | MAMA oligo which will | | |
| | C (SEQ ID NO: 82) | form 500 bp amplicon when | | |
| | | OVPL449 | | |
| | | is incorporated | | |
| oVPL1670 | cgttaaaataggaaaacctttgcttaggt | Targets thyA | LAR_0739 | Y38*Q39SM40L |
| | caaatcgcaAGCTTtatccgaaaa | | | |
| | cagatttagtacctgttcctgtccgat | | | |
| | (SEQ ID NO: 83) | | | |
| oVPL1671 | gctatttcttagataaagtggctgac | Fwd, starts 500 bp upstream | | |
| | (SEQ ID NO: 84) | of site mutated by | | |
| | | oVPL1670 | | |
| oVPL1672 | tttgcttaggtcaaatcgcaagctt | MAMA oligo which will | | |
| | (SEQ ID NO: 85) | form 500 bp amplicon | | |
| | | when oVPL1670 is | | |
| | | incorporated | | |
| oVPL1673 | aaaattggaacatggtgtgacatgga | Rev, starts 500 bp | | |
| | (SEQ ID NO: 86) | downstream of site mutated | | |
| | | by oVPL1670 | | |
| oVPL3694 | acattttctgcattagttgcttgttgagcag | Targets 11993 | LAR_0044 | Q211RG212* |
| | atagcttTCACCggtaagcatcatttt | | | |
| | ccttagcaacagctgagttgtaa | | | |
| | (SEQ ID NO: 87) | | | |
| oVPL3695 | agttcgggcaactgctgatc (SEQ | Fwd, starts 500 bp upstream | | |
| | ID NO: 88) | of site mutated by | | |
| | | oVPL3694 | | |
| oVPL3696 | gcttgttgagcagatagcttTCACC | MAMA oligo which will | | |
| | (SEQ ID NO: 89) | form 500 bp amplicon when | | |
| | | oVPL3694 | | |
| oVPL3697 | taaccgcattgtaaaattcacggtagt | Rev, starts 500 bp | | |
| | (SEQ ID NO: 90) | downstream of site mutated | | |
| | | by oVPL3694 | | |
| oVPL3763 | catacccacgaatccaaattactgagat | Targets fbpA | LAR_0878 | N68*P69* |
| | cccatacaaaTGATAggcggttcc | | | |
| | aactaattttacaatgacaatgcggaaat | | | |
| | (SEQ ID NO: 91) | | | |
| oVPL3764 | ggtgattaatactggctctggattttc | Fwd, starts 500 bp upstream | | |
| | (SEQ ID NO: 92) | of site mutated by | | |
| | | OVPL3763 | | |
| oVPL3766 | gacaacatgaatattattagccgccg | Rev, starts 500 bp | | |
| | (SEQ ID NO: 93) | downstream of site mutated | | |
| | | by oVPL3763 | | |
| oVPL3836 | tggaaccgccTATCA (SEQ | MAMA oligo which will | | |
| | ID NO: 94) | form 500 bp amplicon when | | |
| | | oVPL3763 | | |
| oVPL3796 | aacactatatccagttttacttaattcataa | Targets cmbA | LAR_0958 | P282*N283D |
| | gtatcatCCTATtttactttaacaaca | | | |
| | acagcattactataattgcttcc (SEQ | | | |
| | ID NO: 95) | | | |
| oVPL3797 | tacaagcccttaaagtca (SEQ ID | Fwd, starts 500 bp upstream | | |
| | NO: 96) | of site mutated by | | |
| | | oVPL3796 | | |
| oVPL3798 | ttgttgttaaagtaaaATAGG | MAMA oligo which will | | |
| | (SEQ ID NO: 97) | form 500 bp amplicon when | | |
| | | oVPL3796 | | |
| oVPL3799 | atgttacctcatcagct (SEQ ID | Rev, starts 500 bp | | |
| | NO: 98) | downstream of site mutated | | |
| | | by oVPL3796 | | |
| oVPL3802 | ttgatatttggctaggtcagaccaatcagt | Targets cyclic- | LAR_0983 | P58*Q59* |
| | agtcgtttATTACgtacttgcttcatc | phosphodiesterase (cidi) | | |
| | cttattagtctggaccattggcgt (SEQ | | | |
| | ID NO: 99) | | | |
| oVPL3837 | tggtagggaagtaatttcaatccc | Fwd, starts 500 bp upstream | | |
| | (SEQ ID NO: 100) | of site mutated by | | |
| | | oVPL3802 | | |
| OVPL3838 | tcactggcaagtactgaatgttgg | Rev, starts 500 bp | | |
| | (SEQ ID NO: 101) | downstream of site mutated | | |
| | | by oVPL3802 | | |
| oVPL3839 | gtcagaccaatcagtagtcgtttATT | MAMA oligo which will | | |
| | AC (SEQ ID NO: 102) | form 500 bp amplicon when | | |
| | | oVPL3802 | | |
| OVPL3808 | aatttttatacgcttgattcttagaagttaag | Targets pilP | LAR_0989 | N162*Q163* |
| | tttcctCATCAataataagtaatataa | | | |
| | tcaagcattgatctttcataaa (SEQ | | | |
| | ID NO: 103) | | | |
| oVPL3809 | tctaacttttgaagtaattc (SEQ ID | Fwd, starts 500 bp upstream | | |
| | NO: 104) | of site mutated by | | |
| | | oVPL3808 | | |
| oVPL3810 | gaagttaagtttcctCATCA | MAMA oligo which will | | |
| | (SEQ ID NO: 105) | form 500 bp amplicon when | | |
| | | oVPL3808 | | |
| oVPL3811 | gactggccttttgtaatt (SEQ ID | Rev, starts 500 bp | | |
| | NO: 106) | downstream of site mutated | | |
| | | by oVPL3808 | | |
| OVPL3814 | aaagtgaagttacaattggtgtatttaaatt | Targets slpA | LAR_1193 | V102*Q103* |
| | ttgtaatCATCAgtcattagttttaatt | | | |
| | acatttttatttctgttagtaa (SEQ ID | | | |
| | NO: 107) | | | |
| oVPL3815 | actatcagaacccgttag (SEQ ID | Fwd, starts 500 bp upstream | | |
| | NO: 108) | of site mutated by | | |
| | | oVPL3814 | | |
| oVPL3816 | attaaaactaatgacTGATG | MAMA oligo which will | | |
| | (SEQ ID NO: 109) | form 500 bp amplicon when | | |
| | | oVPL3814 | | |
| oVPL3817 | gaatacttgctgactagt (SEQ ID | Rev, starts 500 bp | | |
| | NO: 110) | downstream of site mutated | | |
| | | by oVPL3814 | | |
| oVPL3850 | ccaaccatcttttactggtgttgatatctct | Targets apf1 | LAR_0410 | D82E G83* |
| | gttgactACTACgttactgagtttgct | | | E84* |
| | ttttgatccgtggcttgttgagt (SEQ | | | |
| | ID NO: 111) | | | |
| OVPL3851 | ggattgacggtaatcattgtctac | Fwd, starts 500 bp upstream | | |
| | (SEQ ID NO: 112) | of site mutated by | | |
| | | oVPL3850 | | |
| OVPL3983 | tgttgatatctctgttgactACTAC | MAMA oligo which will | | |
| | (SEQ ID NO: 113) | form 500 bp amplicon when | | |
| | | oVPL3850 | | |
| oVPL3984 | ggcttatagccgatgtgca (SEQ ID | Rev, starts 500 bp | | |
| | NO: 114) | downstream of site mutated | | |
| | | by oVPL3850 | | |
| oVPL3856 | tgcattagctgcgttttgagcgttgtattctt | Targets autolysin (auto) | LAR_1284 | P99* K100* |
| | gaatttACTACtcgctcttgatgatta | | | |
| | acttttgaccaacgtaaatctt (SEQ | | | |
| | ID NO: 115) | | | |
| oVPL3857 | ggtgctgttacagcttagta (SEQ ID | Fwd, starts 500 bp upstream | | |
| | NO: 116) | of site mutated by | | |
| | | oVPL3856 | | |
| oVPL3858 | gttaatcatcaagagcgaGTAGT | MAMA oligo which will | | |
| | (SEQ ID NO: 117) | form 500 bp amplicon when | | |
| | | oVPL3856 | | |
| oVPL3859 | ctcgacctatacctgtcgaa (SEQ | Rev, starts 500 bp | | |
| | ID NO: 118) | downstream of site mutated | | |
| | | by oVPL3856 | | |
| oVPL3939 | agcgaatcccatttagttggtacaaagtt | Targets cnBp | LAR_0284 | M76RG77* |
| | agcttttaaTTATCtctttttagcaact | | | |
| | gctttaccaagatctacttcaaag | | | |
| | (SEQ ID NO: 119) | | | |
| oVPL3752 | tccgaatgaattatctggcggac | Fwd, starts 500 bp upstream | | |
| | (SEQ ID NO: 120) | of site mutated by | | |
| | | oVPL3939 | | |
| oVPL3754 | gctggatcttgttcactagaaacat | Rev, starts 500 bp | | |
| | (SEQ ID NO: 121) | downstream of site mutated | | |
| | | by oVPL3939 | | |
| oVPL3940 | taaagcagttgctaaaaagaGATA | MAMA oligo which will | | |
| | A (SEQ ID NO: 122) | form 500 bp amplicon when | | |
| | | oVPL3939 | | |
| oVPL3993 | AAACagatcttggtaaagcagttgc | cnBp protospacer sequence | | |
| | taaaaagatG (SEQ ID | | | |
| | NO: 123) | | | |
| oVPL3994 | AAAACatctttttagcaactgctttac | cnBp protospacer sequence | | |
| | caagatct (SEQ ID NO: 124) | | | |
| OVPL3848 | gtttcccaaaacacctatacctgaaaatg | Targets cat*. Incorporates | N/A | |
| | cNttttcNNNNtcNattattocttgg | random bases at stop codon | | |
| | acttcatttactgggtttaacttaa (SEQ | and bases encoding A138, | | |
| | ID NO: 125) | S140, and S142 | | |
| oVPL3996 | gtttcccaaaacacctatacctgaaaatg | ΔcmbA barcode | N/A | |
| | cTttttcGACGtcTattattccttgga | | | |
| | cttcatttactgggtttaacttaa (SEQ | | | |
| | ID NO: 126) | | | |
| oVPL3997 | gtttcccaaaacacctatacctgaaaatg | Δautolysin barcode | N/A | |
| | cTttttcTTGCtcGattattccttgga | | | |
| | cttcatttactgggtttaacttaa (SEQ | | | |
| | ID NO: 127) | | | |
| OVPL3998 | gtttcccaaaacacctatacctgaaaatg | Δapf1 barcode | N/A | |
| | cGttttcTATTtcTattattccttgga | | | |
| | cttcatttactgggtttaacttaa (SEQ | | | |
| | ID NO: 128) | | | |
| oVPL3999 | gtttcccaaaacacctatacctgaaaatg | A11993 barcode | N/A | |
| | cCttttcGACAtcTattattccttgga | | | |
| | cttcatttactgggtttaacttaa (SEQ | | | |
| | ID NO: 129) | | | |
| oVPL4000 | gtttcccaaaacacctatacctgaaaatg | ΔfbpA barcode | N/A | |
| | cTttttcCTTAtcCattattccttgga | | | |
| | cttcatttactgggtttaacttaa (SEQ | | | |
| | ID NO: 130) | | | |
| oVPL4001 | gtttcccaaaacacctatacctgaaaatg | ΔsrtA barcode | N/A | |
| | cGttttcTATCtcTattattccttgga | | | |
| | cttcatttactgggtttaacttaa (SEQ | | | |
| | ID NO: 131) | | | |
| oVPL4002 | gtttcccaaaacacctatacctgaaaatg | ΔslpA barcode | N/A | |
| | cTttttcAGCAtcTattattccttgga | | | |
| | cttcatttactgggtttaacttaa (SEQ | | | |
| | ID NO: 132) | | | |
| oVPL4003 | gtttcccaaaacacctatacctgaaaatg | ΔpilP barcode | N/A | |
| | cTttttcAGTTtcAattattccttgga | | | |
| | cttcatttactgggtttaacttaa (SEQ | | | |
| | ID NO: 133) | | | |
| oVPL4005 | gtttcccaaaacacctatacctgaaaatg | Δcidi barcode | N/A | |
| | cTttttcGTGTtcTattattccttgga | | | |
| | cttcatttactgggtttaacttaa (SEQ | | | |
| | ID NO: 134) | | | |

oVPL: Van Pijkeren Lab oligonucleotide identification number. Bold indicates recombineering oligonucleotide; uppercase bases indicate mismatches with wild-type sequence. All recombineering oligonucleotides target the lagging strand.
^{#}: Fwd: forward; Rev: reverse; oligo: oligonucleotide. The locus tags refer to the fully annotated and closed genome of L. reuteri JCM1112 and can be found on https://www.ncbi.nlm.nih.gov.
* LAR_#### refer to closed reference genome Limosilactobacillus reuteri JCM1112.
^{†}: * indicates nonsense mutation.

### Bacterial Survival of Putative Adhesion Mutants Following Gastrointestinal Transit.

Fifty-eight six-week-old male C57BL/6J mice were purchased from The Jackson Laboratory (Bar Harbor, ME). Prior to the start of the experiment, the animals were adjusted to the new environment for one week. The animals were individually housed in an environmentally controlled facility with a 12 h light and 12 h dark cycle. Food (standard chow; LabDiet, St. Louis, MO) and water were provided ad libitum. Mice (n=5-8/treatment group) were gavaged for two consecutive days with 100 μl phosphate-buffered saline (PBS) suspension containing ˜109 CFU/ml of chloramphenicol-resistant L. reuteri adhesion mutants. Fresh fecal samples were collected 15, 27, and 39 h after the last oral administration and weighed. The fecal material was resuspended in PBS to 100 mg/ml and plated on MRS agar plates containing 5 μg/ml chloramphenicol. Cell viability counts were normalized per 108 CFU administered L. reuteri.

### Inactivation of Nine Genes Encoding Putative Adhesins in a Single Genetic Background.

L. reuteri VPL4018 is a derivative of L. reuteri VPL1014 in which the gene encoding sortase was inactivated (25). Strain VPL4018 was transformed with pVPL2032, which encodes RecT; this strain was subjected to ssDNA recombineering to generate a double mutant, followed by generating a triple mutant etcetera until we inactivated nine total genes to yield a nonuple mutant (Table 4). The mutations were achieved in the following order: 1) ΔsrtA, 2) ΔslpA, 3) ΔcmbA, 4) Δauto, 5) Δapf1, 6) ΔpilP, 7) Δ11993, 8) ΔfbpA, and 9) Δcidi.

Construction of L. reuteri ΔcnBp Single Mutant.

We were unable to identify a recombinant genotype for cnBp with single-stranded DNA recombineering. To increase selective power, we employed CRISPR-Cas9-assisted recombineering (25). We first generated a plasmid encoding gRNA targeting cnBp. Briefly, pVPL3115 (25) was digested with Eco31I (Thermo Fisher Scientific followed by gel purification (Thermo Fisher Scientific, FERK0701). A pair of complementary oligonucleotides (oVPL3993-oVPL3994) identical to the 30-bp target region of cnBp were annealed to digested pVPL3115. DNA was mixed at a 1:1 molar ratio followed by overnight ligation, pellet paint precipitation and transformation in L. lactis MG1363. We confirmed by sequence analysis insertion of the cnBp protospacer, yielding pCRISPR-cnBp. L. reuteri VPL3187 harboring pVPL3004 (25) and pVPL3016 (24, 25) was then co-transformed with oVPL3939 and pCRISPR-cnBp as described previously (25) to generate ΔcnBp (M76RG77*). Following genotype confirmation, ΔcnBp was passaged in MRS until Cm-, Em-, and Tet-phenotypes were confirmed by restored antibiotic sensitivity. Plasmid loss was subsequently validated by PCR, and the subsequent strain VPL4379 (ΔcnBp) was confirmed by Sanger sequencing.

### Complementation of Genes Encoding Adhesion Proteins.

For complementation, the target gene was cloned into a high-copy expression vector via ligase cycle reaction (LCR) (14). Briefly, we amplified the backbone of pJP028 (derived from pNZ8048) with primer pair oVPL1286-oVPL1408. We placed each gene under the control of the L. reuteri pMutL promoter, a promoter located upstream of the gene encoding MutL, which is involved in DNA repair (33, 86). We amplified pMutL with oVPL736 and oVPL737 using pSIP411:pMutL-ThyA as the template. Each gene encoding putative adhesion protein was amplified from their start to stop codons using oligonucleotides listed in Table 3. First, cmbA amplified with oVPL4033 and oVPL4034 was ligated to pMutL and the pJP028 backbone with bridging oligonucleotides oVPL4035, oVPL4036, and oVPL4037. Next, 5 μL of the LCR mixture was directly transformed into E. coli EC1000 and plated on LB plates supplemented with 300 μg/ml erythromycin. Insertion of pMutL and cmbA into pJP028 was confirmed via PCR with oVPL329 and oVPL363, followed by Sanger sequencing. The resulting plasmid (pVPL31467) was amplified with oVPL737 and oVPL1286 to insert the remaining genes encoding putative adhesion proteins. Each complementation plasmid was then amplified with oVPL309 and oVPL310 to omit the cat cassette from the pJP028 backbone, which would otherwise interfere with our ability to distinguish strains via their barcodes. Finally, each complementation plasmid was electroporated into L. reuteri in which the corresponding gene was inactivated.

### Adhesion Assay on HT-29 Human Colon Cancer Cells.

L. reuteri wild-type and its derivatives were tested for their ability to adhere to the human colorectal adenocarcinoma cell line, HT-29 (ATCC HTB-38). For bacterial cell preparation, overnight (˜16-hour) cultures were diluted to an OD600=0.1 in MRS and cultured to OD600=1.0. One ml of bacterial culture was harvested by centrifugation (1 min at 21,130×g). The cell pellet was washed once in 1 ml Dulbecco's phosphate buffered saline (DPBS), centrifuged as above, and resuspended in 1 ml PBS. To prepare the epithelial cells, cells were seeded at 2×104 cells/well (passages 4-9) and grown to 100% confluency in a 24-well plate (Biolite, Thermo Scientific™, 12-556-006). For the adhesion assay, the cell culture medium was removed, and the cells were washed with 1 ml PBS. In a volume of 250 μl, ˜5×106 bacterial cells were added to the monolayer (MOI=5:1). After 30 minutes of incubation at 37° C. and 5% CO2, the cell layer was gently washed with PBS five times to remove non-adherent bacteria. After the final wash, cells were lysed by adding ice-cold dH2O and the cell layer was disrupted with a 1 ml pipette tip. The remaining adhered bacteria in the suspension were vigorously vortexed, serial diluted and enumerated by standard plate counts. Adhesion to HT-29 cells was calculated as percent of adhered bacteria relative to the total bacteria added. We obtained six biological replicates with three technical replicates each.

### Adhesion Assay on Human Colon Cancer Enteroids Monolayers.

All single mutant strains, complemented strains, and barcoded controls were grown overnight (˜16 h) in MRS broth supplemented with erythromycin (5 μg/ml) as needed. One day prior to the assay, enteroids monolayers were washed once and media was replaced with antibiotic-free DMEM/F12. Overnight cultures were diluted to OD600=0.1, and grown to OD600=1.0 at which point 1 ml of cells were harvested by centrifugation (21,130×g for 1 min). Cells were resuspended in 1 ml pre-warmed DMEM/F12 (Thermo fisher) and cell concentration was quantified by plating serial dilutions. Mixtures of ˜1:1:1:1: . . . 1 ratios of single mutant strains, complemented strains, or barcoded controls were prepared by mixing equal volumes of each in a single tube. The resulting mixtures were then diluted to achieve a final MOI of 5, and plated to quantify total cell concentration. Next, 250 μL of each mixture was added to 6 wells each of enteroid monolayers for 3 technical replicates of washed wells and unwashed control wells per group. After 1 h incubation at 37° C. in a humidified atmosphere of 5% CO2, the bacterial suspensions were carefully aspirated, and three wells of each mixture were washed three times with pre-warmed PBS. The remaining three cells per mixture serve as unwashed controls. After the last wash, enteroid cells were lysed by adding 1 ml (or 750 μL for unwashed wells) of ice-cold dH2O. The monolayers were then disrupted with a pipette tip to create a suspension of enteroid cells and bacteria before transferring the suspension to a 1.5 ml tube. After vortexing vigorously for 40 seconds, adhered bacterial cells were quantified by plating serial dilutions. Total DNA from the cell suspensions was then extracted by bead beating. Briefly, 300 μL of zirconia glass beads (BioSpec) and the cell suspensions were added to 2 ml microvials before loading onto a bead beater (BioSpec, Mini-Beadbeater-16) and beat for 3 minutes at 30 s intervals, with 30 s on ice in between beating. Cell lysate was harvested by centrifugation at (21,130×g for 1 min). DNA was extracted from the cell lysates with the Qiagen DNeasy Blood and Tissue Kit according to the manufacturer's instructions. DNA samples were then prepared for NGS sequencing. Nonuple mutant was also competed with rifampicin-resistant VPL4216 (L. reuteri::rpoB(H488R)). Construction of VPL4216 was described previously (24, 36). The procedure was identical to the single mutant competition experiment except cells were plated on MRS with or without rifampicin supplementation (25 μg/ml) to differentiate between nonuple and VPL4126. The resulting ratio of adhered VPL4126 to nonuple cells was determined by comparing cell counts on MRS plates supplemented with rifampicin (VPL4126 count) and total cells on MRS plates. Nonuple cell counts were calculated by subtracting the rifampicin-resistant CFU from the total CFU count. Experiments were performed with 3 biological replicates with 3 technical replicates each.

### Library Preparation of Adhesion Competition DNA Samples.

DNA samples were prepared by PCR enrichment (Roche, KAPA HiFi) with oligonucleotides that add Illumina adapters (oVPL4155, oVPL4156, oVPL4157, and oVPL4158). Oligonucleotides oVPL4157 and oVPL4158 have six N's in between the annealing sequence and the Illumina adapters to add sequence diversity at the ends of each amplicon. After confirming the absence of primer-dimers via gel electrophoresis, enriched samples were purified with a GeneJet purification kit (ThermoFisher, FERK0701). Next, 10 μL of each enriched sample was used as template for index PCR with KAPA HiFi (8 cycles). Oligonucleotides used for sample enrichment and indexing are listed in Table 5, and were designed to distinguish samples from all biological and technical replicates. Indexed samples were then purified and quantified (Qubit fluorometric quantification; Life Technologies).

TABLE 5
Oligonucleotides used for library preparation.

| Oligo | | |
|---|---|---|
| Name | Sequence (5′-3′)^{†} | Target/comment^{#} |
| oVPL4155 | acactctttccctacacgacgctcttccgatctcctgctgt | Fwd, internal to cat gene with |
| | aataatgggtagaagg (SEQ ID NO: 135) | Illumina adapter |
| oVPL4156 | gtgactggagttcagacgtgtgctcttccgatcttggact | Rev, internal to cat gene with |
| | cctgtaaagaatgacttca (SEQ ID NO: 136) | Illumina adapter |
| oVPL4157 | acactctttccctacacgacgctcttccgatctNNNN | Fwd, internal to cat gene with |
| | NNcctgctgtaataatgggtagaagg (SEQ ID | Illumina adapter; “N′s” indicate |
| | NO: 137) | degenerate nucleotides |
| oVPL4158 | gtgactggagttcagacgtgtgctcttccgatctNNN | Rev, internal to cat gene with |
| | NNNtggactcctgtaaagaatgacttca (SEQ ID | Illumina adapter; “N′s” indicate |
| | NO: 138) | degenerate nucleotides |
| oVPL4210 | AATGATACGGCGACCACCGAGAT | Fwd, stem and index i5 for illumina |
| | CTACACcggaagaaACACTCTTTCCCT | sequencing_1 |
| | ACACGACGCT (SEQ ID NO: 139) | |
| oVPL4211 | CAAGCAGAAGACGGCATACGAGA | Rev, stem and index i7 for illumina |
| | TttcttccgGTGACTGGAGTTCAGACG | sequencing_1 |
| | TGTGC (SEQ ID NO: 140) | |
| oVPL4212 | AATGATACGGCGACCACCGAGAT | Fwd, stem and index i5 for illumina |
| | CTACACgacaccaaACACTCTTTCCCT | sequencing_2 |
| | ACACGACGCT (SEQ ID NO: 141) | |
| oVPL4213 | CAAGCAGAAGACGGCATACGAGA | Rev, stem and index i7 for illumina |
| | TttggtgtcGTGACTGGAGTTCAGACG | sequencing_2 |
| | TGTGC (SEQ ID NO: 142) | |
| oVPL4214 | AATGATACGGCGACCACCGAGAT | Fwd, stem and index i5 for illumina |
| | CTACACacaactggACACTCTTTCCCT | sequencing_3 |
| | ACACGACGCT (SEQ ID NO: 143) | |
| oVPL4215 | CAAGCAGAAGACGGCATACGAGA | Rev, stem and index i7 for illumina |
| | TccagttgtGTGACTGGAGTTCAGACG | sequencing_3 |
| | TGTGC (SEQ ID NO: 144) | |
| oVPL4216 | AATGATACGGCGACCACCGAGAT | Fwd, stem and index i5 for illumina |
| | CTACACcggtactaACACTCTTTCCCT | sequencing_4 |
| | ACACGACGCT (SEQ ID NO: 145) | |
| oVPL4217 | CAAGCAGAAGACGGCATACGAGA | Rev, stem and index i7 for illumina |
| | TtagtaccgGTGACTGGAGTTCAGACG | sequencing_4 |
| | TGTGC (SEQ ID NO: 146) | |
| oVPL4218 | AATGATACGGCGACCACCGAGAT | Fwd, stem and index i5 for illumina |
| | CTACACactcacacACACTCTTTCCCT | sequencing_5 |
| | ACACGACGCT (SEQ ID NO: 147) | |
| oVPL4219 | CAAGCAGAAGACGGCATACGAGA | Rev, stem and index i7 for illumina |
| | TgtgtgagtGTGACTGGAGTTCAGACG | sequencing_5 |
| | TGTGC (SEQ ID NO: 148) | |
| oVPL4220 | AATGATACGGCGACCACCGAGAT | Fwd, stem and index i5 for illumina |
| | CTACACctagacgaACACTCTTTCCCT | sequencing_6 |
| | ACACGACGCT (SEQ ID NO: 149) | |
| oVPL4221 | CAAGCAGAAGACGGCATACGAGA | Rev, stem and index i7 for illumina |
| | TtcgtctagGTGACTGGAGTTCAGACG | sequencing_6 |
| | TGTGC (SEQ ID NO: 150) | |
| oVPL4222 | AATGATACGGCGACCACCGAGAT | Fwd, stem and index i5 for illumina |
| | CTACACataggtcgACACTCTTTCCCT | sequencing_7 |
| | ACACGACGCT (SEQ ID NO: 151) | |
| oVPL4223 | CAAGCAGAAGACGGCATACGAGA | Rev, stem and index i7 for illumina |
| | TcgacctatGTGACTGGAGTTCAGACG | sequencing_7 |
| | TGTGC (SEQ ID NO: 152) | |
| oVPL4224 | AATGATACGGCGACCACCGAGAT | Fwd, stem and index i5 for illumina |
| | CTACACtagcagcaACACTCTTTCCCT | sequencing_8 |
| | ACACGACGCT (SEQ ID NO: 153) | |
| oVPL4225 | CAAGCAGAAGACGGCATACGAGA | Rev, stem and index i7 for illumina |
| | TtgctgctaGTGACTGGAGTTCAGACG | sequencing_8 |
| | TGTGC (SEQ ID NO: 154) | |

oVPL: Van Pijkeren Lab oligonucleotide identification number. Bold indicates recombineering oligonucleotide; uppercase bases indicate mismatches with wild-type sequence. All recombineering oligonucleotides target the lagging strand.
^{#}: Fwd: forward; Rev: reverse

Targeted Sequence Analysis from Competition Experiment on Human Enteroid Monolayers.

Sequencing was performed at the University of Wisconsin-Madison Biotechnology Center. Quality and quantity of the finished libraries were analyzed using Agilent Tapestation and Quantus Qubit dsDNA assay. Samples were diluted to 2 nM before sequencing. Paired-end, 150 bp sequencing was performed using the Illumina MiSeq Sequencer and a MiSeq 300 bp (v2) sequencing cartridge. Quality control images were analyzed using MultiQC v1.dev0. Paired-end Illumina sequencing reads were merged and filtered with PEAR (Paired-End reaD mergeR) using default settings (87). Phred quality scores (Q scores) were used to compute the total number of expected errors (E) for each merged read, and reads exceeding an Emax of 1 were removed. The ratios of strains before and after the assay were determined by the number of filtered reads that matched each barcode compared to the total filtered reads within each sample. Reads that corresponded to barcodes not used in in the present examples, which altogether constituted less than 4.1% of the total reads in each sample, were excluded from calculations.

Construction of NonupleΔthyA::rpoB(H488R) Harboring IL-22 Expression Vector.

We inactivated thyA in the Nonuple mutant with oVPL1670 by ssDNA recombineering as described previously (24, 33). NonupleΔthyA mutants were selected by plating serial dilutions onto modified MRS without beef extract supplemented with trimethoprim (40 μg/ml) and thymidine (50 μg/ml) (33, 88). NonupleΔthyA was confirmed by MAMA PCR (85, 89) with oVPL1671, oVPL1672, and oVPL1673, followed by Sanger sequencing. A rifampicin-resistant derivative was generated to quantify L. reuteri in fecal samples following GI transit. Briefly, a mutation in rpoB (H488R) was introduced into NonupleΔthyA with oVPL236 by ssDNA recombineering, as described previously, resulting in NonupleΔthyA::rpoB(H488R) (33). NonupleΔthyA::rpoB(H488R) was subsequently transformed with pVPL31134 (pCtl-ThyA) and pVPL31464 (pIL-22-ThyA) to assess biological functionality in a mouse model of diet-induced metabolic syndrome. Construction of pVPL31134 and pVPL31464 was described previously (33, 34). Henceforth, we will refer to NonupleΔthyA::rpoB(H488R) and LRΔthyA::rpoB(H488R) harboring pCtl-ThyA as Non-Ctl and WT-Ctl, respectively; and NonupleΔthyA::rpoB(H488R) and LRΔthyA::rpoB(H488R) harboring pIL-22-ThyA as Non-IL22 and LR-IL22, respectively. IL-22 production by LR-IL22 and Non-22 was detected by ELISA, as described previously (33).

### High-Fat and High-Sugar-Induced Fatty Liver Disease Model.

Sixty 6-week-old male C57BL/6J mice were purchased from The Jackson Laboratory (Bar Harbor, ME). The animals were housed (n=4 per cage) in an environmentally controlled facility with a 12 h light and 12 h dark cycle for 8 weeks on a high-fat diet (TD.08811 45% kcal diet (21% milk fat, 34% sucrose); Envigo) before treatment. Animals were provided with food and water ad libitum. Mice consumed the Western diet for 16 weeks; at week 8, mice (n=12 per group) were gavaged daily for the remaining 8 weeks with 100 μl PBS (sham group), or bacteria containing 1010 CFU/ml of one of the following: VPL31134 (LRΔthyA::rpoB(H488R) harboring pThyA control vector) (34), VPL31168 (LRΔthyA::rpoB(H488R) harboring pIL22-ThyA) (34), VPL31497 (NonupleΔthyA::rpoB(H488R) harboring pThyA control vector), or VPL31498 (NonupleΔthyA::rpoB(H488R) harboring pIL22-ThyA). Body weight was monitored, and fresh fecal samples were collected every other week. The fecal material was resuspended in PBS to 100 mg/ml and plated on MRS agar plates containing 25 μg/ml rifampicin or 5 μg/mL erythromycin. Plaque forming units (PFU) were quantified in fecal material at week 7 and the endpoint. Cell viability counts and PFU were normalized per 108 CFU. At the endpoint (16 weeks), mice were euthanized by CO2 prior to tissue sampling.

### Blood Plasma Isolation.

Fifteen hours after the last gavage and directly after the mice were euthanized, 500 μl blood per mouse was collected via cardiac puncture and mixed with EDTA at a final concentration of 5 mM. Plasma was isolated from the whole-blood sample by centrifugation at 4° C., 8,150×g for 7 min, and the plasma fraction was stored at −80° C. until use.

### Quantification of Alanine Transaminase (ALT) and Aspartate Transaminase (AST).

ALT and AST in blood plasma were quantified via colorimetric assays according to the manufacturer's instructions (Sigma-Aldrich, MAK052-1KT and MAK055-1KT). The final absorbances were measured in a microplate reader (SpectraMax Plus 384; Molecular Devices). A standard curve was generated using JMP software to calculate ALT and AST concentrations.

### Liver Triglyceride Analysis.

Liver triglycerides (TGs) were quantified following the Jouihan method (90). Total lipids were extracted from 100-300 mg of frozen liver tissue in ethanolic KOH at 55° C. for 16 h. Triglyceride content was determined by colorimetric analysis using free glycerol reagent (Sigma-Aldrich; F6428) and glycerol standard (Sigma-Aldrich; G7793). A standard curve was generated using JMP software to calculate liver triglyceride concentrations.

### Statistics.

Data representation was performed using DataGraph (version 4.3) software (Visual Data Tools, Inc., Chapel Hill, NC, USA). Statistical comparisons were performed using a paired t test, one-way analysis of variance, and Tukey's honestly significant difference test (HSD) (JMP Pro software, version 14.0.0). Three biological replicates were performed for all in vitro studies. All samples were included in the analyses, and experiments were performed without blinding.

### Results

To identify target genes for inactivation to yield a strain with reduced adhesive ability, we first focused on genes encoding sortase-dependent proteins (SDPs). We identified a single sortase gene and eight genes encoding sortase-dependent proteins (SDPs). We excluded pseudogenes (LAR_1193-1192 and LAR_0089) and genes that are unlikely to play a role in adhesion, which are LAR_0813 and LAR_0903. LAR_0903 has low homology (28% sequence identity) to YggS, a protein important for Vitamin B6 metabolism (1, 2), whereas LAR_0813 is annotated as an amidase.

To identify genes that encode non-sortase-dependent proteins that could play a role in adhesion, we used the genome of L. reuteri JCM1112 as a reference because it is a closed genome and only contains 3 SNPs when compared to L. reuteri 6475 (3). We searched the JCM1112 genome for adhesion proteins homologs that have been functionally characterized and determined to play a role in adhesion in other gram-positive bacteria. Our analyses revealed 5 genes: aggregation promoting factor (apf1) (4, 5), fibronectin binding protein (fbpA) (6), surface layer protein (slpA) (7), collagen binding protein (cnBp) (8, 9), and autolysin (10) (Table 2). Our analysis yielded a total of 10 genes with a putative role in cell and/or mucus attachment. (Table 2).

### Development of a Mutant Library Tagging Method.

To tag recombinant strains, we developed a barcoding system. A chromosomal barcoding system in a gut symbiont will open the door to multiplex the functional characterization of user-defined recombinants. To accomplish this, we first designed a barcoding target. Here, we chose to integrate into the L. reuteri chromosome a derivative of the gene encoding chloramphenicol resistance (cat) that contained an in-frame stop codon to yield VPL4011. To generate barcodes in the L. reuteri chromosome, we applied single-stranded DNA recombineering using a degenerate oligonucleotide (oVPL3848) that, when incorporated, repairs the stop codon in the cat gene and generates mutations at wobble base positions creating unique tags. To map the distribution of mutations generated, we sequenced the cat gene of 96 chloramphenicol-resistant colonies; we observed 81 unique barcodes and 15 repeated barcodes. Adenosines were overrepresented at positions 1 and 6 (which are the original bases at those positions), and position 3, which is within the codon that replaces the stop codon. Bases at positions 2 and 4 are evenly distributed, while guanine is underrepresented at position 5. Notably, only 8 out of 20 amino acids were represented in the 96 transformants as replacements for the stop codon. Thus, without the use of a purified recombineering oligonucleotide and optimization studies, our approach provides a robust means to create nearly 100 unique chromosomal tags in a single step.

We chose this barcoding method because it placed us in the position to screen for recombinants within a pool of cells that have successfully undergone a recombineering event, i.e. are chloramphenicol resistant. We hypothesized that this approach will recover recombinants at a higher frequency compared to a conventional recombineering approach that does not employ antibiotic selection. To test this, we simultaneously co-transformed strain VPL4011 with oligonucleotides oVPL3848 and oVPL236, which—upon successful incorporation—repair the stop codon in the cat gene and generate a mutation in the gene encoding RNA polymerase B, which renders the cells resistant to rifampicin. Plating of the dual-transformation on MRS agar supplemented with chloramphenicol or rifampicin revealed recombination efficiencies of 2.47±0.385% and 1.25±0.385% relative to total CFU, respectively. Subsequent patch plating of 100 dual-transformed colonies from chloramphenicol plates onto rifampicin plates resulted in 6% of colonies that were resistant to both antibiotics. Therefore, we recovered approximately 5-fold more recombinants when we screen a pool of cells that has successfully undergone a recombineering event. This placed us in the position to apply this approach to inactivate genes putatively encoding adhesins.

### Single-Mutant Library Construction and Growth Characterization.

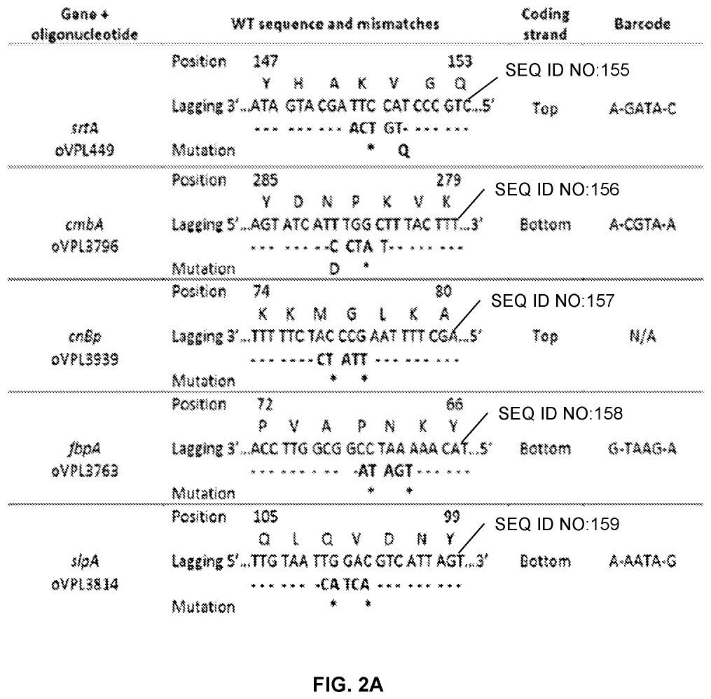

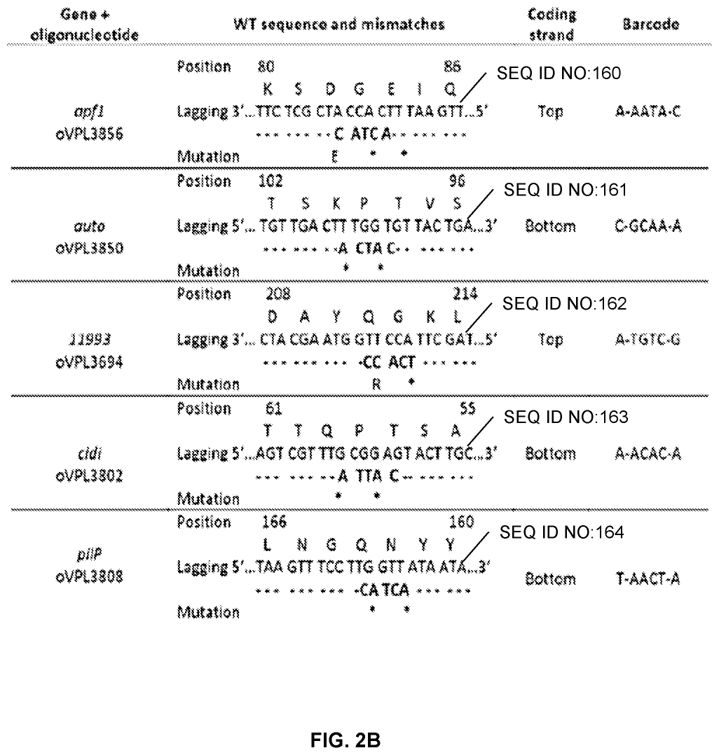

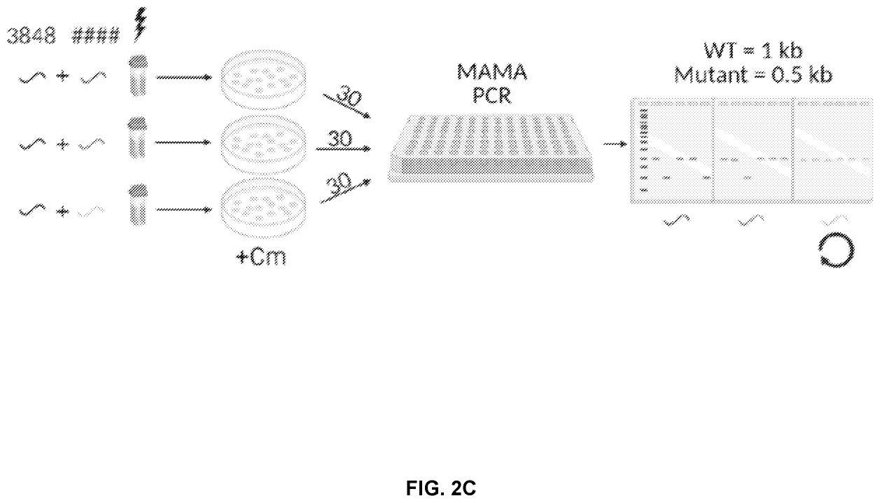

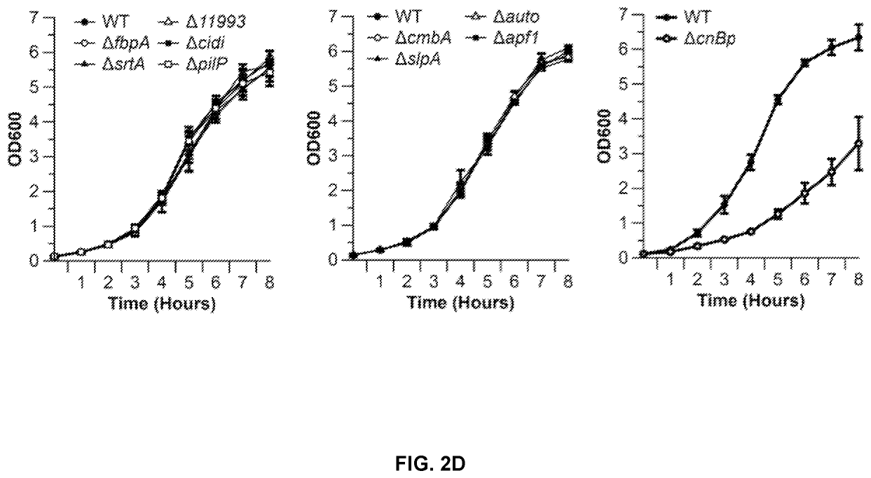

We applied the dual-recombineering concept to generate nine adhesion protein mutants, each with a unique barcode (FIGS. 2A and 2B, Table 4). To optimize the mutant screening process, we developed a scheme to efficiently identify adhesin mutants (FIG. 2C). Briefly, we dual-transformed the barcoding oligonucleotide oVPL3848 and one of three oligonucleotides targeting different adhesins into VPL4011.

After plating transformants on agar supplemented with chloramphenicol, we screened thirty colonies from each transformation via MAMA PCR in a 96-well plate. We observed that with a dual-recombineering method in which we transform VPL4011 with oVPL3848 and a recombineering oligonucleotide targeting a putative adhesion mutant, we were often able to recover at least one recombinant per adhesin target per 30 CFU screened. Recombineering transformations that did not result in mutant recovery after screening 30 CFU were carried over to the next round of transformations until the desired genotype was recovered (FIG. 2C). Nine adhesin mutants were recovered using this method, all with unique barcodes that restored cat*. One mutant, ΔcnBp, was not recoverable with this method and was instead achieved with CRISPR-Cas9-assisted recombineering (11, 12).

After confirming all mutants and identifying their barcodes, we performed basic characterization of each mutant to better inform the design of our biotherapeutic delivery vehicle. We tested each single mutant for growth or fitness defects that would impede the ability of a sequential mutant to deliver biotherapeutics. Any mutants that conferred a growth defect would be excluded from the sequential mutant. Growth analysis indicates that ΔcnBp exhibited a growth defect and was thus excluded from further analysis (FIG. 2D). Growth rates (data not shown, p>0.05) were calculated from the resulting growth curves, and no differences were observed between the remaining 9 mutants and the chloramphenicol-resistant wild-type control (VPL4052) (FIG. 2D). Next, we tested the adhesin mutants for gastrointestinal survival in mice.

Inactivation of Genes Encoding Surface Adhesins does not Impact Gastrointestinal Survival.

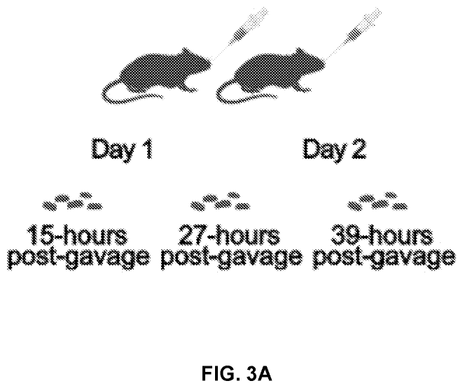

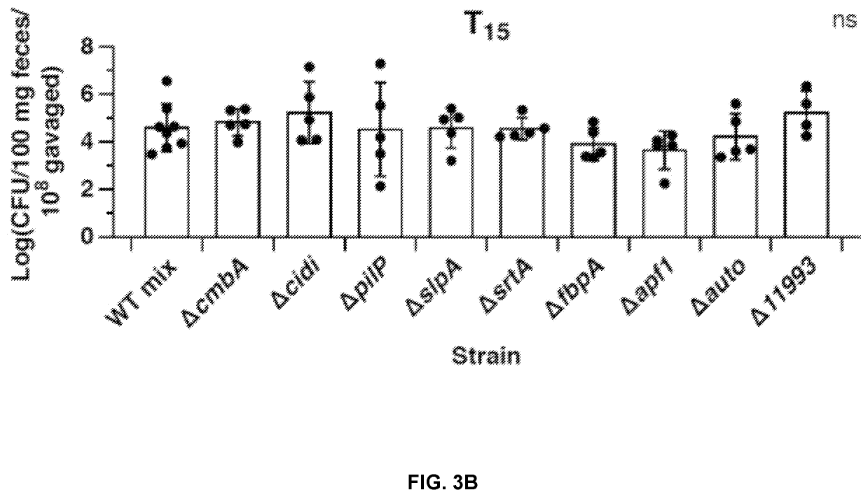

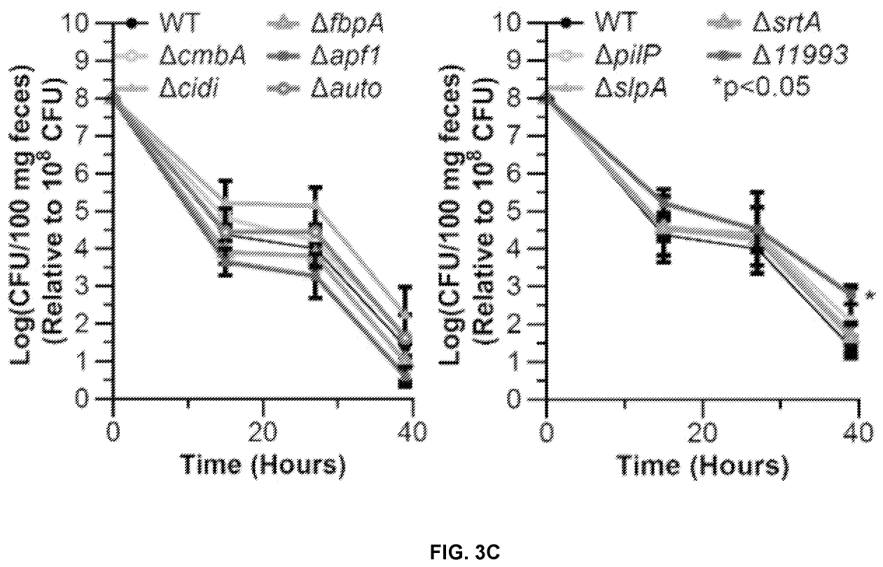

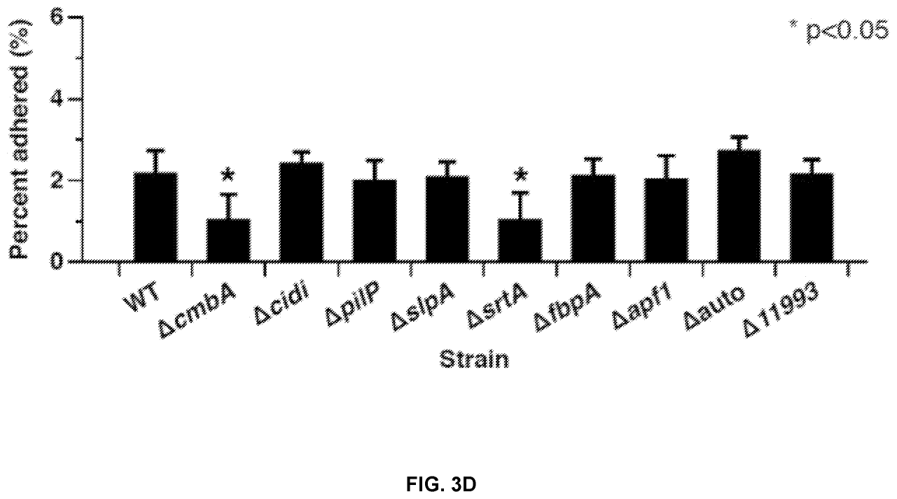

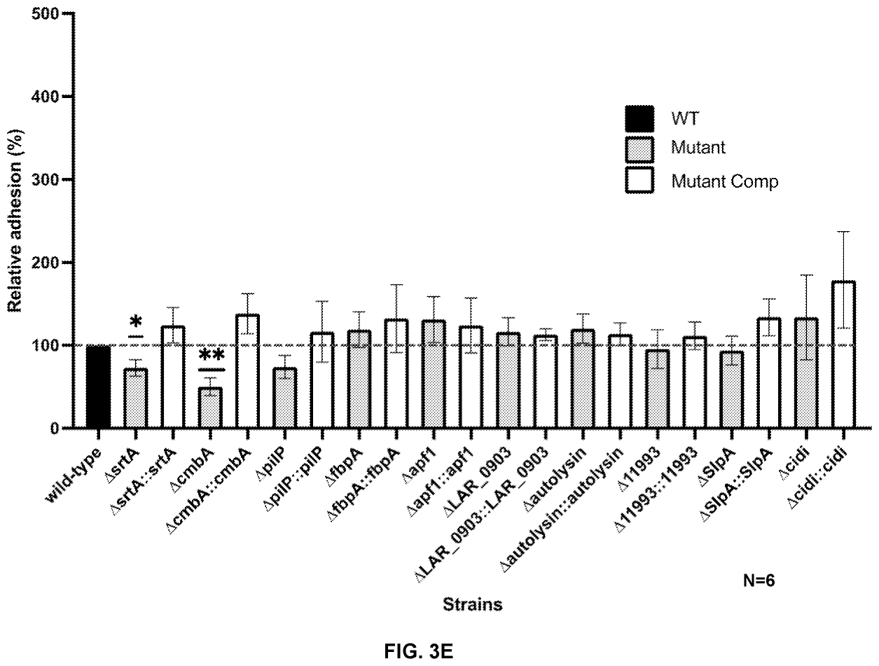

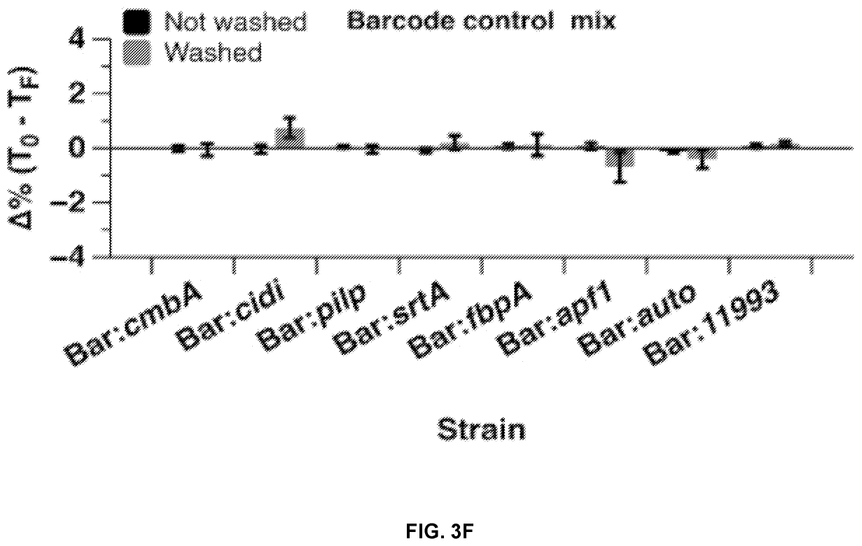

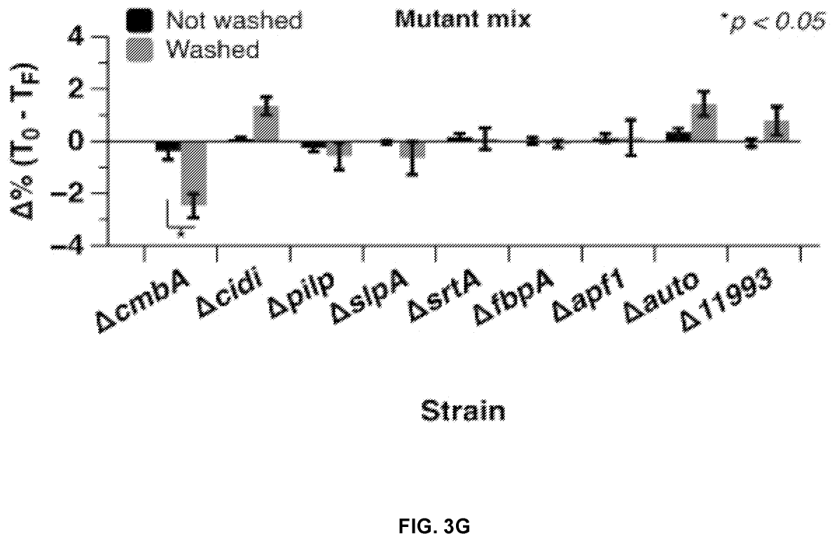

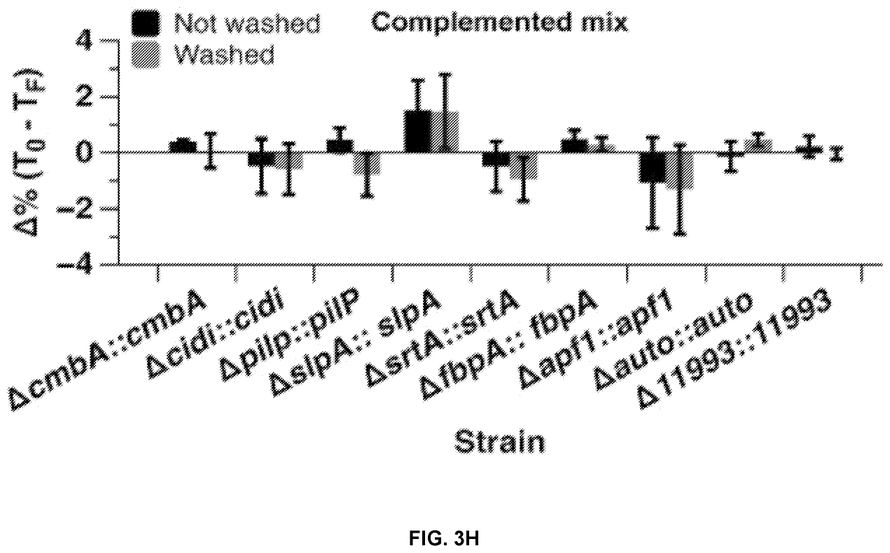

To determine gastrointestinal survival, we administered each mutant and VPL4052 to mice for two consecutive days with 108 CFU per day (n=5-8 mice/group). After the second gavage, we recovered L. reuteri and its mutants from the fecal material after 15 hours, 27 hours, and 39 hours (FIG. 3A). Average recovery at 15 h ranged from 104 CFU/100 mg feces (ΔfbpA and Δapf1) to 105 CFU/100 mg feces (ΔcmbA, Δcidi, and Δhmp_11993), with VPL4052 recovery at 105 (CFU/100 mg feces) (FIG. 3B). None of the differences in recovery compared to VPL4052 were significant (p>0.5).

We also tested the ability of each adhesin mutant to thrive in the gut by tracking persistence. Less than 1-log reduction in recovery was observed across all groups except for Δauto (which did not show a decrease) between the 15 h and 27 h time points (FIG. 3C). At 39 h, a 2-3-log decrease was observed from 27 h for each group, except for Δ11993, which decreased by just under 2-log (1.73±0.528), which was significantly different from the other groups (p<0.05). Overall, the persistence results combined with the survival results indicate that gut fitness is not negatively affected by individual adhesin mutations compared to the wild-type control. We next tested the mutants for their ability to adhere to HT-29 human colon cancer cells.

Inactivation of cmbA and srtA Causes Defective Adhesion to Human Colon Cancer Cells.

All nine single mutants and VPL4052 (chloramphenicol-resistant control) were grown to mid-log phase, and ˜5×106 CFU were added to HT-29 tissue culture monolayers to yield a MOI of 5:1. After 30 minutes of incubation, cells were washed with PBS to remove non-adherent bacteria and adherent cells were harvested and plated to calculate percent adhesion, as described previously (13). Strains ΔcmbA (an SDP) and ΔsrtA (sortase) adhered significantly less (1.06±0.244% and 1.06±0.337%, respectively) to HT-29 compared to VPL4052 (2.19±0.297%, p<0.02), while the remaining seven mutants adhered to HT-29 at levels comparable to VPL4052 (p>0.05; FIG. 3D) (FIG. 3E). However, we cannot eliminate the possibility that these seven mutants play a role in adhesion based solely on this model.

HT-29 cells produce little to no mucus (14). Human intestinal organoid-derived epithelial monolayers (HIODEM model), however, contain mucus-producing goblet cells in addition to enterocytes and antigen sampling M cells (15-18). To test the role of these adhesins in adhering to monolayers derived from human colon cancer enteroids, we designed a competition experiment. After growing each mutant to mid-log phase (OD600=1.0), we harvested cells and mixed them together in 1:1:1: . . . . :1 ratio. We used mixtures of complemented mutants and barcoded VPL4011 as controls. The mixtures (5×106 CFU total) were then added to monolayers (MOI=5:1) and co-incubated for 1 hour in DMEM/F12. After washing unadhered bacteria, cells were plated to determine percent adherence of the mixtures, which did not indicate a significant difference in adherence between the groups (1.14±0.66% barcode control vs 2.01±1.3% mutant mix vs. 1.34±0.13% complemented mix, p>0.3).

To gain insight into the adhesive ability of each strain, we determined the relative abundance of each mutant. First, samples were lysed by bead beating, and total DNA was harvested and prepared for next-generation sequencing targeting the cat gene barcodes. We compared the percentage of reads corresponding to each barcode in the mixtures just before adding them to the enteroid monolayers (T0) to the percentage of barcode reads recovered from the adhesion assay (TF) (FIG. 3F). A positive percentage change indicates that the relative proportion of a mutant increased in the population, while a negative percentage change indicates a decrease in relative proportion. Mixtures added to unwashed cells (not washed) served as controls to compare percent changes between TF and T0 of the washed cells. As expected, the ratio of strains in the barcode control mix (FIG. 3F) did not change during the experiment (percent changes were all between ±0.70%), indicating that each strain within the control mixture was equally capable of adhering to the monolayers. Importantly, the ratio of strains in the unwashed control also did not change (all percent changes between ±0.1%), which serves as an indication for the sensitivity and accuracy of our sequencing method (FIGS. 3F-3G). For the mutant mixture, only ΔcmbA exhibited a significant decrease in ratio compared to the ΔcmbA unwashed control (˜2.46±0.46% vs. 0.46±0.33%, respectively, p<0.05) (FIG. 3G). Two mutants, Δcidi (1.34±0.34%) and Δauto (1.43±0.46%) increased (though not significantly) in relative ratio following the adhesion assay, which corresponds to the results of the adhesion assay on HT-29 cells (FIG. 3D and FIG. 3G). Unexpectedly, ΔsrtA did not exhibit a decrease in relative proportion between TF and T0 (0.10±0.4%). The complemented mixture exhibited higher variance than the barcode and mutant mixes, which we expect is due to variation in the effect of constitutive expression of each complemented gene (FIG. 3H). Despite this, the ratios of each strain between the washed and not washed samples are very similar, and with the exceptions of Δapf1, ΔslpA and ΔsrtA, most TF ratios remained very similar to the respective T0 ratios (changed less than ±0.5%) (FIG. 3H). Altogether, these data confirmed that ΔcmbA is deficient in adhesion compared to the other mutants tested; and that our barcode detection method is sufficiently sensitive and consistent for this application.

Although seven out of nine adhesin mutants do not impact adhesion to in vitro cancer cell lines, it cannot be excluded that there is a role for bacterium-host interactions in the human GI tract. Thus, we continued to construct a nonuple mutant containing nine adhesion protein mutations to serve as our biotherapeutic delivery platform.

### HT-29 Adhesion Competition Assay.

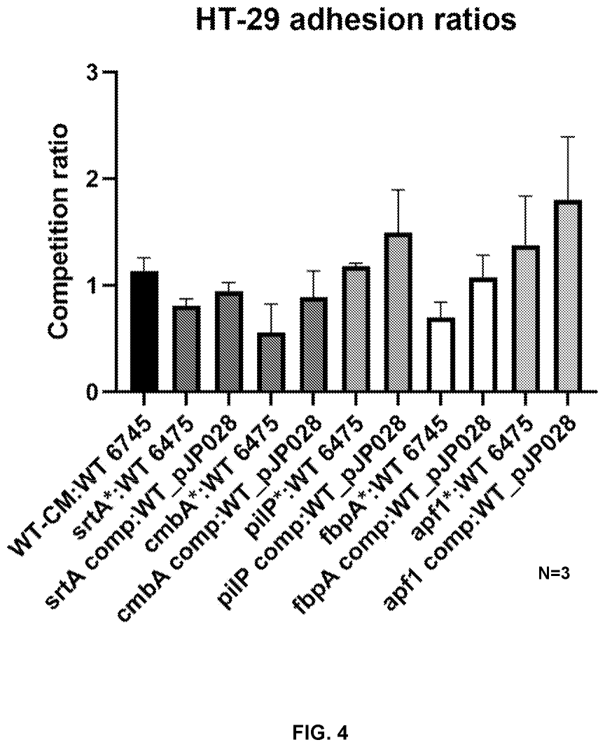

HT-29 cells (ATCC HTB-38) were cultivated in Dulbecco's modified Eagle's medium containing 4.5 g/L glucose with L-glutamine and sodium pyruvate (Corning, 10-013-CV) and supplemented with 10% (v/v) fetal bovine serum (Sigma-Aldrich, 12306C). Each cell line was propagated for no more than 20 passages. For each assay, cells were seeded into a 12-well plate (Corning, 3513) and grown at 37° C. in 5% CO₂ until reaching 100% confluence. Overnight (˜16-hr) cultures of L. reuteri 6475 wild-type and its derivatives were sub-cultured to OD₆₀₀=0.1 in MRS (supplemented with erythromycin 5 μg/mL as needed) and cultured to OD₆₀₀=1.0. For each strain, cultures were harvested by centrifugation (2 min, 15,900×g) and the cell pellet was resuspended in 1 mL Dulbecco's phosphate buffered saline (Gibco, 14190144). Bacterial mixtures were subsequently prepared by mixing two strains in a 1:1 ratio. The wild-type mix contained WT 6475 (VPL1014) mixed with a chloramphenicol-resistant mutant (VPL4052); the mutant mix contained WT 6475 mixed with an individual adhesion mutant; and the complement mix contained WT 6475 harboring a pJP028 control plasmid mixed with the complemented mutant strain. Each mixture was diluted 20-fold to achieve an approximate cell concentration of ˜2×10⁷ CFU/mL, which was verified by enumeration on MRS agar. After removing the medium from the HT-29 cells and washing once with pre-warmed PBS, 500 μL of the diluted bacterial suspensions was added to the surface of the monolayers (MOI=5:1). For each mixture, the assay was performed in technical triplicates. After 30 minutes of incubation at 37° C. and 5% CO2, the monolayers were gently washed with PBS five times to remove non-adherent bacteria. Subsequently, ice-cold dH₂O was added and the wells were scraped with a 1 mL pipette tip to lyse the epithelial cells and collect the cell mixtures. The remaining adhered bacteria in the suspension were vigorously vortexed for 1 minute and serially diluted in PBS for enumeration on MRS agar. Bacteria were plated on regular MRS and MRS supplemented with chloramphenicol 5 μg/mL (for complement mix, plates also contained erythromycin 5 μg/mL) to distinguish between strains in each mixture. Adhesion competition ratios were calculated as the ratio of the mutant or the complemented mutant (all chloramphenicol-resistant) to the wild-type strain. Three biological replicates were performed for each adhesion mutant and complemented mutant pair. Individual adhesion mutant strains, complementation plasmids, and combinatorial adhesion mutant strains are shown in Tables 6, 7, and 8, respectively. Results are shown in FIG. 4.

TABLE 6
Individual adhesion mutant strains.

| | Gene target | Locus | VPL number |
|---|---|---|---|
| 1 | srtA | LAR_0227 | VPL4363 |
| 2 | slpA | LAR_1193 | VPL4362 |
| 3 | cmbA | LAR_0958 | VPL4359 |
| 4 | autolysin | LAR_1284 | VPL4367 |
| 5 | apf1 | LAR_0410 | VPL4365 |
| 6 | pilP | LAR_0989 | VPL4361 |
| 7 | 11993 | LAR_0044 | VPL4368 |
| 8 | fbpA | LAR_0878 | VPL4364 |
| 9 | cidi (cyclic phosphodiesterase) | LAR_0983 | VPL4360 |
| 10 | LAR_0903 | LAR_0903 | VPL4382 |

TABLE 7
Complementation plasmids.

| | Plasmid | VPL number |
|---|---|---|
| 1 | pJP028 derivative, srtA complementation plasmid | pVPL31516 |
| 2 | pJP028 derivative, slpA complementation plasmid | pVPL31515 |
| 3 | pJP028 derivative, cmbA complementation plasmid | pVPL31467 |
| 4 | pJP028 derivative, autolysin complementation | pVPL31518 |
| | plasmid | |
| 5 | pJP028 derivative, apf1 complementation plasmid | pVPL31519 |
| 6 | pJP028 derivative, pilP complementation plasmid | pVPL31514 |
| 7 | pJP028 derivative, 11993 complementation plasmid | pVPL31520 |
| 8 | pJP028 derivative, fbpA complementation plasmid | pVPL31517 |
| 9 | pJP028 derivative, cidi complementation plasmid | pVPL31522 |
| 10 | pJP028 derivative, LAR_0903 complementation | pVPL31570 |
| | plasmid | |

TABLE 8
Combinatorial adhesion mutant strains.

| | | |
|---|---|---|
| VPL4366 | Nonuple | Mutant with genes 1-9 inactivated |
| VPL4386 | Decuple | Mutant with genes 1-10 inactivated |

### A Nonuple Mutant has a Reduced Capacity to Adhere to Enteroid Monolayers.

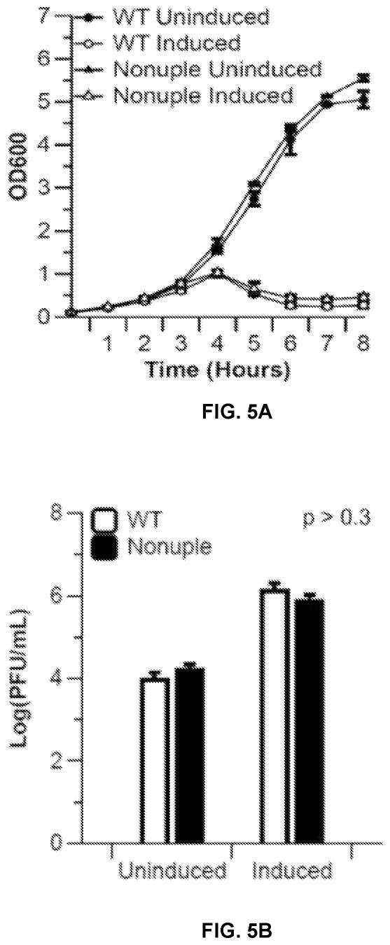

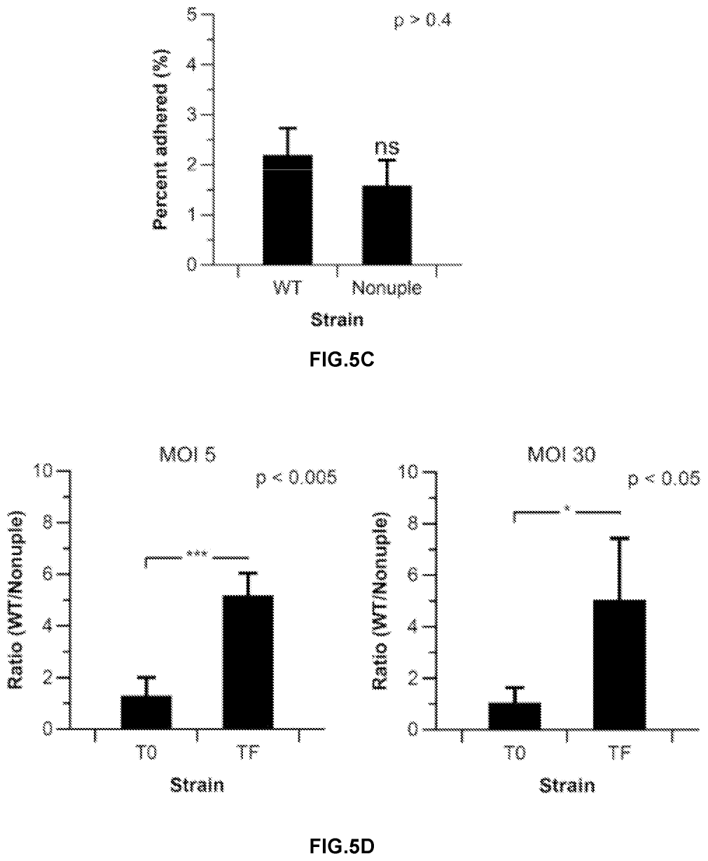

An untagged combinatorial mutant was obtained by sequentially transforming each adhesion mutant's recombineering oligonucleotide into a single strain. A double mutant, triple mutant, quadruple mutant, and so on were obtained until all nine mutations were in a single strain, resulting in the nonuple mutant (VPL4366). Because we aim to employ the nonuple as our delivery vehicle platform, we wanted to ensure that the combined mutations did not confer a growth defect or impact our delivery mechanism of phage-mediated lysis (19). We first characterized the growth of the nonuple mutant (FIG. 5A), which had a similar doubling-time compared to the wild-type (1.15±0.03 vs. 0.99±0.05 doublings/hr, respectively; p>0.05). Next, we characterized the phage production by the nonuple mutant. Mitomycin C induction of phage resulted in lysis of both wild-type and the nonuple mutant, and the lysis patterns overlapped (FIG. 5A). At the endpoint of the growth analysis (T8), supernatant of each culture was harvested, and phage were quantified (FIG. 5B). Phage levels of wild-type and the nonuple derivative were similar ((3.96±0.179 log(PFU/mL) vs 4.20±0.143 log(PFU/mL), respectively; p>0.3)). Also, basal level phage production was similar ((6.12±0.193 log(PFU/mL) and 5.86±0.152 log(PFU/mL), for wild-type and recombinant strain, respectively; (p>0.3)).

Next, we tested the ability of the nonuple derivative to adhere to HT-29 cells. Each strain (˜5×106 CFU) was co-incubated with a confluent monolayer of HT-29 (MOI 5:1). Our results revealed that the nonuple variant did not adhere significantly less to HT-29 monolayers compared to the wild-type (1.59±0.500% vs. 2.20±0.297%, respectively; p>0.4) (FIG. 5C). However, using the enteroid assay model we observed that the control strain significantly outcompeted the nonuple derivative (FIG. 5D). In this experimental setup, the rifampicin-resistant control strain VPL4216 and the nonuple-mutant were mixed in a 1:1 ratio and plated for quantification before the addition to the monolayers (FIG. 5D, T0). The nonuple mutant and VPL4216 mixture was then added to enteroid monolayers to yield a total MOI of 5:1 or 30:1, and co-incubated for 1 hour. After the unadhered bacteria were removed by washing, we determined that the control strain outcompeted the adhesive ability of the nonuple strain by 5-fold regardless of the MOI (FIG. 5B, TF). In conclusion, our data collectively placed us in the position to further develop the nonuple variant as a therapeutic platform. To test proof-of-principle, we engineered the nonuple mutant to produce murine Interleukin-22 (IL-22) for delivery to mice that developed fatty liver disease in response to a high-sugar high-fat diet.

### Nonuple Releases Intracellularly Accumulated IL-22 Via Phage-Mediated Lysis.

To test the potential of the nonuple mutant to deliver biotherapeutics compared to wild-type, we engineered the nonuple mutant to intracellularly accumulate Interleukin-22 (IL-22). We previously demonstrated that L. reuteri secreting IL-22 to mice fed a high-fat diet alleviated diet-induced metabolic syndrome and fatty-liver disease (20). In addition, we demonstrated the efficacy of phage-mediated delivery of IL-22 by wild-type L. reuteri in two different models: alcohol-induced liver disease and the increased survival of irradiated mice (21, 22). This collectively provided a foundation to test the impact of phage-mediated delivery of IL-22 by the nonuple derivative on markers of diet-induced metabolic syndrome, including fatty liver, in a mouse model of diet-induced obesity.

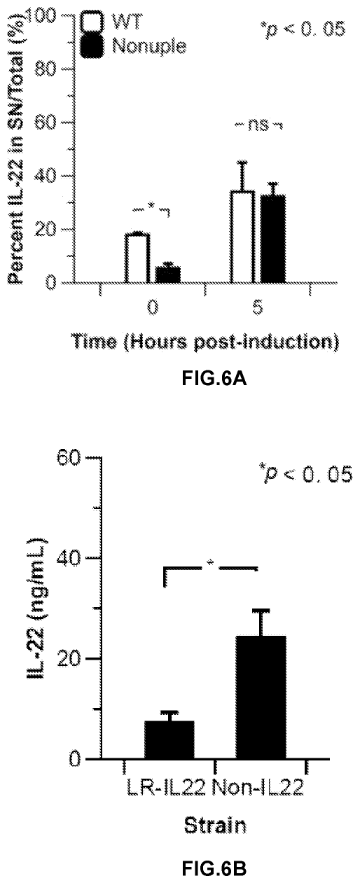

To stably maintain the IL-22 expression plasmid in the nonuple mutant, we disrupted the gene encoding thymidylate synthase (thyA), as described previously (19, 23), resulting in nonupleΔthyA. A plasmid encoding ThyA will be stably maintained in this genetic background without the need for antibiotic selection. To facilitate the recovery of L. reuteri from mouse fecal material, we modified the rpoB gene to confer rifampicin resistance, as described previously (11, 19), resulting in NonupleΔthyA::rpoB(H488R). We then transformed NonupleΔthyA::rpoB(H488R) with pVPL31134 (pCtl-ThyA) and pVPL31464 (pIL-22-ThyA) (19, 22). Wild-type L. reuteri (LRΔthyA::rpoB(H488R)) strains harboring pCtl-ThyA and pIL-22-ThyA were constructed in a previous study and were used as controls (22). Henceforth, we will refer to NonupleΔthyA::rpoB(H488R) and LRΔthyA::rpoB(H488R) harboring pCtl-ThyA as Non-Ctl and WT-Ctl, respectively; and NonupleΔthyA::rpoB(H488R) and LRΔthyA::rpoB(H488R) harboring pIL-22-ThyA as Non-IL22 and LR-IL22, respectively. Before testing the nonuple mutant in the animal disease model, phage-mediated release of IL-22 by LR-IL22 and Non-IL22 was compared. Phage-activation of LR-IL22 and Non-IL22 was achieved by mitomycin C induction, and total IL22 levels were determined in the supernatants at zero-hours post-induction (T0) and five-hours post-induction (T5). The percent of IL-22 released into the supernatant compared to the total IL-22 produced by LR-IL22 was higher than by Non-IL22 at T0 (18.0±0.61% vs. 5.5±1.7%, respectively, p<0.05), but IL-22 release at T5 between the two strains was very similar (34.07±11.00% vs. 32.2±4.8%, respectively, p>0.5). (FIG. 6A). Total IL-22 production at T5 by induced Non-IL22 (24.2±5.5 ng/mL) was higher than induced LR-IL22 (7.2±2.1 ng/mL, p<0.05) (FIG. 6B). No IL-22 was detected from LR-Ctl or Non-Ctl samples. These data indicate that both strains release IL22 at similar levels due to phage-mediated lysis, though Non-IL22 produces ˜3-fold more IL-22 than LR-IL22, which is possibly due to a difference in IL-22 plasmid stability. We next compared phage-mediated delivery of IL-22 by LR-IL22 and Non-IL22 in mice fed a high-fat, high-sugar diet.

Survival of NonupleΔthyA::rpoB(H488R) and LRΔthyA::rpoB(H488R)+/−IL-22 in Diet-Induced Metabolic Disease in Mice.

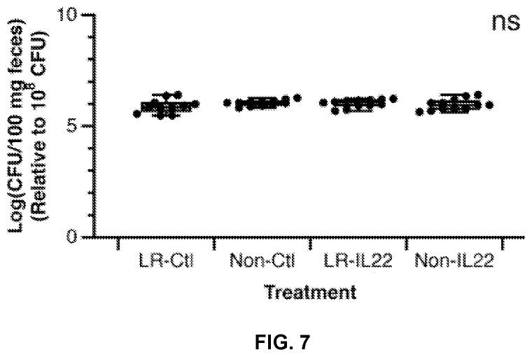

Mice were fed a high-sugar high-fat diet (Teklad, TD.08811) for eight weeks before the start of treatment. At week nine, treatment began by administering CFU of each strain to mice (n=11-12/group) daily and continued for 8 weeks. One group was administered phosphate-buffered saline (PBS) as a sham control. Fecal CFU and body weights were monitored every two weeks throughout the treatment portion of the experiment. Bacterial survival rates were nearly identical across the groups at 10⁶ CFU/100 mg feces expressed as averages of all samples (CFU was not detectable from the sham group) (FIG. 7).

### Discussion

As a novel biocontainment measure, we engineered L. reuteri to reduce the organism's ability to persist and colonize the intestinal tract. We constructed ten adhesin mutants which we tracked by a unique barcode in the chromosome. Nine out of ten selected adhesin mutants demonstrated no effect on growth or in vivo survival, and several mutants adhered significantly less to intestinal epithelial cells. These phenotypes informed the development of a nonuple mutant, which was significantly deficient in its ability to adhere to human colonoid monolayers.

To enable the direct comparison of the adhesion mutants in in vitro and in vivo settings, we devised a barcoding scheme that generates unique tags in the chloramphenicol acetyltransferase (cat) gene of each mutant. Our design can theoretically generate a total of 3,904 unique barcodes yielding a functional cat gene. However, in our initial screen, we observed that 81 out of 96 recombinant genotypes were unique, which suggests a bias towards either bases at the wobble positions or codons that replace the stop codon. One possibility is that recombination with the non-optimized oVPL3848 biased the results by failing to evade the DNA mismatch repair system (MMR) at the wobble base positions. At wobble base positions the degenerate oligonucleotide introduces a single mismatch that is most likely detected by MMR, as only five adjacent mismatches have demonstrated full avoidance of MMR in L. reuteri (24). It is also plausible that certain amino acids may not restore cat function, as we observed codons that replaced the stop codon translate to only 8/20 amino acids. If the limited number of amino acids at the stop codon position relates to the functionality or folding of Cat rather than recombination efficiency of oVPL3848, the total potential barcodes would decrease to 1,536. Despite the potential limitation of only a few amino acids restoring Cat, 85% of the 96 oVPL3848 transformants sequenced contained unique barcodes. Additionally, the barcode is within a gene conferring antibiotic resistance, and it is also possible that random mutations may arise in the barcode during growth, resulting in background sequencing reads in downstream experiments. However, we observed that less than 5% of total reads in our competition experiment were non-mutant barcode sequences, indicating that at least in L. reuteri, this is not a significant concern. Therefore, regardless of the amino acid determinants of cat function and without modification, our dual-recombineering method is more than capable of generating small, barcoded libraries in a quick and efficient manner.

We employed our dual-recombineering approach to generate the adhesin single mutant after demonstrating an increase in the recovery of recombinant colonies by ˜5-fold compared to the traditional, single-recombineering method (24). We expect that the increase in recombination efficiency is due to selecting from a population that already experienced one recombination event, which increases the likelihood for a second event. This is possibly due to a variation in RecT expression within the population, and we expect that this approach analogously increased the recovery rates of our adhesion mutants. This data provides proof-of-concept for our dual-recombineering approach that could be expanded for use in other non-domesticated gut symbiont strains in which the basal recombineering efficiency is too low to recover recombinants. With our approach, it may be possible to overcome low recombineering efficiencies via co-selection, perhaps by targeting rpoB (an essential and conserved gene in which mutations confer rifampicin resistance) (63) in place of cat*. Following mutant construction, we observed ΔcmbA and ΔsrtA exhibited lower adhesion to HT-29 cells compared to wild-type. The present examples indicate that not all the tested proteins affect adhesion to HT-29 cells, though they could still play a role in adhesion in vivo due to different conditions and unknown expression levels of these putative adhesins in vitro compared to in vivo. HT-29 cells are well characterized, form tight junctions, and have a typical apical brush border, and are useful for studying adhesion and host-microbe interactions (64, 65). However, a limitation of HT-29 cells is that they do not completely reflect the in vivo environment because they lack diversity in cell type and structure. Therefore, we predict these putative adhesins will play a role in adhesion in conditions that are more physiologically similar to those encountered in vivo.

An emerging model to study probiotic-host interactions is the human intestinal organoid-derived epithelial monolayer (HIODEM) model. Monolayers derived from enteroids are composed of a variety of cell types including mucus-producing goblet cells, Paneth cells, and hormone-secreting enteroendocrine cells, which create a more in vivo like environment than cell lines (66). However, the culturing of enteroid monolayers is more costly and laborious than traditional cell lines (67). We instead devised a competition experiment in which we assayed a mixture of all mutants for adherence in a manner similar to previous adhesin studies (68, 69). A pooled adhesion competition assay also allows for the direct comparison of the adhesion ability of each mutant relative to each other. In this competition experiment, the CmbA mutant was the only mutant significantly less able to adhere compared to the other mutants. We hypothesize this is due to the presence of the other mutants that may complement the adhesive ability of the sortase mutant and/or the other adhesins through unknown interactions between the strains. Importantly, variation across the replicates was generally very low in this experiment, validating our barcoding method to distinguish these strains with a high level of sensitivity in a pooled competition assay. We expect this method to be broadly applicable to study small mutant libraries in L. reuteri and other bacteria in which recombineering can be applied. Notably, we observed that the nonuple mutant is significantly deficient in adhering to enteroid monolayers in competition with wild-type, as are several individual mutants.

Lastly, we tested the delivery of the nonuple derivative compared to the wild-type in a model of high-fat high-sugar (HFHS) induced metabolic syndrome, which we recently used to demonstrate that secretion of IL-22 by L. reuteri alleviates fatty liver disease (32). We also had shown that phage-mediated delivery of IL-22 by LR-IL22 increased survival of mice exposed to total body irradiation (TBI) (34). We observed that the level of recovery of the strains used in the present examples was ˜2-log lower than that observed in a concurrent HFD study. One difference between our concurrent studies in the HFD model is the inclusion of the thymidylate synthase mutation (ΔthyA). Here, we employed thyA as an auxotrophic marker to maintain the IL-22 expression construct in the absence of antibiotics in the growth medium (33, 74). However, our survival results suggest that despite the complementation of thyA in the IL-22 expression plasmid, ΔthyA results in a fitness cost in the HFD mice. Studies comparing the survival of LRΔthyA harboring the thyA complementation plasmid in HFD mice to wild-type L. reuteri can be performed. Future studies we will evaluate the therapeutic potential of Non-IL22 in mice exposed to total body irradiation, in which LR-IL22 previously demonstrated success (34).

These examples provide proof-of-concept for the removal of adhesin proteins as a biocontainment measure in bacterial biotherapeutics.

### REFERENCES

1. Leventhal D S, Sokolovska A, Li N, Plescia C, Kolodziej S A, Gallant C W, Christmas R, Gao J-R, James M J, Abin-Fuentes A, Momin M, Bergeron C, Fisher A, Miller P F, West K A, Lora J M. 2020. Immunotherapy with engineered bacteria by targeting the STING pathway for anti-tumor immunity. Nature Communications 11(1):2739.
2. Yap Y A, Mariño E. 2021. Dietary SCFAs Immunotherapy: Reshaping the Gut Microbiota in Diabetes, p. 499-519. In Islam, MdS (ed.), Diabetes: from Research to Clinical Practice: Volume 4. Springer International Publishing, Cham.
3. Gniadek T J, Augustin L, Schottel J, Leonard A, Saltzman D, Greeno E, Batist G. 2020. A Phase I, Dose Escalation, Single dose trial of oral attenuated Salmonella typhimurium containing human IL-2 in patients with metastatic gastrointestinal cancers. J Immunother 43:217-221.
4. Pangilinan C R, Lee C-H. 2019. Salmonella-based targeted cancer therapy: Updates on a promising and innovative tumor immunotherapeutic strategy. Biomedicines 7.
5. Hassan R, Alley E, Kindler H, Antonia S, Jahan T, Honarmand S, Nair N, Whiting C C, Enstrom A, Lemmens E, Tsujikawa T, Kumar S, Choe G, Thomas A, McDougall K, Murphy A L, Jaffee E, Coussens L M, Brockstedt D G. 2019. Clinical response of live-attenuated, Listeria monocytogenes expressing mesothelin (CRS-207) with chemotherapy in patients with malignant pleural mesothelioma. Clin Cancer Res 25:5787-5798.
6. Park O, Wang H, Weng H, Feigenbaum L, Li H, Yin S, Ki S H, Yoo S H, Dooley S, Wang F-S, Young H A, Gao B. 2011. In vivo consequences of liver-specific interleukin-22 expression in mice: Implications for human liver disease progression. Hepatology 54:252-261.
7. Jiang R, Wang H, Deng L, Hou J, Shi R, Yao M, Gao Y, Yao A, Wang X, Yu L, Sun B. 2013. IL-22 is related to development of human colon cancer by activation of STAT3. BMC Cancer 13:59.
8. Jiang R, Tan Z, Deng L, Chen Y, Xia Y, Gao Y, Wang X, Sun B. 2011. Interleukin-22 promotes human hepatocellular carcinoma by activation of STAT3. Hepatology 54:900-909.
9. Zheng Y, Li T. 2018. Interleukin-22, a potent target for treatment of non-autoimmune diseases. Human Vaccines & Immunotherapeutics 14:2811-2819.
10. Herich R, Levkut M. 2012. Lactic acid bacteria, probiotics and immune system. Veterinarni Medicina 47:169-180.
11. Maldonado Galdeano C, Cazorla S I, Lemme Dumit J M, Vélez E, Perdigón G. 2019. Beneficial Effects of Probiotic Consumption on the Immune System. ANM 74:115-124.
12. Engevik M A, Ruan W, Esparza M, Fultz R, Shi Z, Engevik K A, Engevik A C, Ihekweazu F D, Visuthranukul C, Venable S, Schady D A, Versalovic J. 2021. Immunomodulation of dendritic cells by Lactobacillus reuteri surface components and metabolites. Physiological Reports 9:e14719.
13. Lee J W, Chan C T Y, Slomovic S, Collins J J. 2018. Next-generation biocontainment systems for engineered organisms. Nat Chem Biol 14:530-537.
14. Mandell D J, Lajoie M J, Mee M T, Takeuchi R, Kuznetsov G, Norville J E, Gregg C J, Stoddard B L, Church G M. 2015. Biocontainment of genetically modified organisms by synthetic protein design. Nature 518:55-60.
15. Chan C T Y, Lee J W, Cameron D E, Bashor C J, Collins J J. 2016. “Deadman” and “Passcode” microbial kill switches for bacterial containment. Nature Chemical Biology 12(2):82-86.
16. Schwartz D J, Langdon A E, Dantas G. 2020. Understanding the impact of antibiotic perturbation on the human microbiome. Genome Medicine 12:82.
17. Abbas Hilmi H T, Surakka A, Apajalahti J, Saris P E J. 2007. Identification of the most abundant Lactobacillus species in the crop of 1- and 5-week-old broiler chickens. Appl Environ Microbiol 73:7867-7873.
18. Dellaglio F, Arrizza S, Ledda A. 1981. Classification of citrate fermenting Lactobacilli isolated from lamb stomach, sheep milk and pecorino romano cheese. Zentralblatt für Bakteriologie Mikrobiologie und Hygiene: I Abt Originale C: Allgemeine, angewandte und ökologische Mikrobiologie 2:349-356.
19. Leser T D, Amenuvor J Z, Jensen T K, Lindecrona R H, Boye M, Møller K. 2002. Culture-independent analysis of gut bacteria: the pig gastrointestinal tract microbiota revisited. Appl Environ Microbiol 68:673-690.
20. Brooks S P J, McAllister M, Sandoz M, Kalmokoff M L. 2003. Culture-independent phylogenetic analysis of the faecal flora of the rat. Can J Microbiol 49:589-601.
21. Salzman N H, de Jong H, Paterson Y, Harmsen H J M, Welling G W, Bos N A. 2002. Analysis of 16S libraries of mouse gastrointestinal microflora reveals a large new group of mouse intestinal bacteria. Microbiology (Reading, Engl) 148:3651-3660.
22. Reuter G. 2001. The Lactobacillus and Bifidobacterium microflora of the human intestine: composition and succession. Curr Issues Intest Microbiol 2:43-53.
23. Frese S A, Benson A K, Tannock G W, Loach D M, Kim J, Zhang M, Oh P L, Heng N C K, Patil P B, Juge N, MacKenzie D A, Pearson B M, Lapidus A, Dalin E, Tice H, Goltsman E, Land M, Hauser L, Ivanova N, Kyrpides N C, Walter J. 2011. The evolution of host specialization in the vertebrate gut symbiont Lactobacillus reuteri. PLOS Genetics 7:e1001314.
24. van Pijkeren J-P, Britton R A. 2012. High efficiency recombineering in lactic acid bacteria. Nucleic Acids Res 40:e76.
25. Oh J-H, van Pijkeren J-P. 2014. CRISPR-Cas9-assisted recombineering in Lactobacillus reuteri. Nucleic Acids Res 42:e131.
26. Zhang S, Oh J-H, Alexander L M, Özçam M, van Pijkeren J-P. 2018. d-Alanyl-d-Alanine ligase as a broad-host-range counterselection marker in vancomycin-resistant lactic acid bacteria. J Bacteriol 200.
27. Sung V, D'Amico F, Cabana M D, Chau K, Koren G, Savino F, Szajewska H, Deshpande G, Dupont C, Indrio F, Mentula S, Partty A, Tancredi D. 2018. Lactobacillus reuteri to treat infant colic: A meta-analysis. Pediatrics 141.
28. Liu Y, Hoang T K, Taylor C M, Park E S, Freeborn J, Luo M, Roos S, Rhoads J M. 2021. Limosilactobacillus reuteri and Lacticaseibacillus rhamnosus GG differentially affect gut microbes and metabolites in mice with Treg-deficiency. American Journal of Physiology-Gastrointestinal and Liver Physiology https://doi.org/10.1152/ajpgi.00072.2021.
29. Wang W, Zijlstra R T, Ganzle M G. 2020. Feeding Limosilactobacillus fermentum K9-2 and Lacticaseibacillus casei K9-1, or Limosilactobacillus reuteri TMW1.656 reduces pathogen load in weanling pigs. Front Microbiol 11.
30. Al-Hadidi A, Navarro J, Goodman S D, Bailey M T, Besner G E. 2021. Lactobacillus reuteri in Its biofilm state improves protection from experimental necrotizing enterocolitis. 3. Nutrients 13:918.
31. Fåk F, Bäckhed F. 2012. Lactobacillus reuteri prevents diet-induced obesity, but not atherosclerosis, in a strain dependent fashion in Apoe−/− mice. PLOS ONE 7:e46837.
32. Oh J-H, Schueler K L, Stapleton D S, Alexander L M, Yen C-LE, Keller M P, Attie A D, Pijkeren J-P van. 2020. Secretion of recombinant interleukin-22 by engineered Lactobacillus reuteri reduces fatty liver disease in a mouse model of diet-induced obesity. mSphere 5.
33. Alexander L M, Oh J-H, Stapleton D S, Schueler K L, Keller M P, Attie A D, Pijkeren J-P van. 2019. Exploiting prophage-mediated lysis for biotherapeutic release by Lactobacillus reuteri. Appl Environ Microbiol 85:e02335-18.
34. Zhang X, Fisher R, Hou W, Shields D, Epperly M W, Wang H, Wei L, Leibowitz B J, Yu J, Alexander L M, van Pijkeren J-P, Watkins S, Wipf P, Greenberger J S. 2020. Second-generation probiotics producing IL-22 increase survival of mice after total body irradiation. In Vivo 34:39-50.
35. Hendrikx T, Duan Y, Wang Y, Oh J-H, Alexander L M, Huang W, Stärkel P, Ho S B, Gao B, Fiehn O, Emond P, Sokol H, Pijkeren J-P van, Schnabl B. 2018. Bacteria engineered to produce IL-22 in intestine induce expression of REG3G to reduce ethanol-induced liver disease in mice. Gut gutjnl-2018-317232.
36. Jee-Hwan Oh, Laura M. Alexander, Meichen Pan, Kathryn L. Schueler, Mark P. Keller, Alan D. Attie, Jens Walter, Jan-Peter van Pijkeren. Dietary fructose and short-chain fatty acids induce acetogenesis in the gut symbiont Lactobacillus reuteri that promotes bacteriophage production in a RecA-dependent manner. Cell Host & Microbe in press.
37. Lebeer S, Vanderleyden J, De Keersmaecker S C J. 2010. Host interactions of probiotic bacterial surface molecules: comparison with commensals and pathogens. Nature Reviews Microbiology 8(3):171-184.
38. Ganguli K, Collado M C, Rautava J, Lu L, Satokari R, von Ossowski I, Reunanen J, de Vos W M, Palva A, Isolauri E, Salminen S, Walker W A, Rautava S. 2015. Lactobacillus rhamnosus GG and its SpaC pilus adhesin modulate inflammatory responsiveness and TLR-related gene expression in the fetal human gut. Pediatric Research 77(4):528-535.
39. Bene K P, Kavanaugh D W, Leclaire C, Gunning A P, MacKenzie D A, Wittmann A, Young I D, Kawasaki N, Rajnavolgyi E, Juge N. 2017. Lactobacillus reuteri surface mucus adhesins upregulate inflammatory responses through interactions with innate c-type lectin receptors. Front Microbiol 8.
40. Call E, Klaenhammer T. 2013. Relevance and application of sortase and sortase-dependent proteins in lactic acid bacteria. Front Microbiol 4.
41. Ton-That H, Liu G, Mazmanian S K, Faull K F, Schneewind O. 1999. Purification and characterization of sortase, the transpeptidase that cleaves surface proteins of Staphylococcus aureus at the LPXTG motif. Proc Natl Acad Sci USA 96:12424-12429.
42. Bae T, Schneewind O. 2003. The YSIRK-G/S Motif of Staphylococcal Protein A and its role in efficiency of signal peptide processing. Journal of Bacteriology 185:2910-2919.
43. Jensen H, Roos S, Jonsson H, Rud I, Grimmer S, van Pijkeren J-P, Britton R A, Axelsson L. 2014. Role of Lactobacillus reuteri cell and mucus-binding protein A (CmbA) in adhesion to intestinal epithelial cells and mucus in vitro. Microbiology, 160:671-681.
44. Muñoz-Provencio D, Pérez-Martinez G, Monedero V. 2010. Characterization of a fibronectin-binding protein from Lactobacillus casei BL23. Journal of Applied Microbiology 108:1050-1059.
45. Hsueh H-Y, Yueh P-Y, Yu B, Zhao X, Liu J-R. 2010. Expression of Lactobacillus reuteri Pg4 collagen-binding protein gene in Lactobacillus casei ATCC 393 increases its adhesion ability to Caco-2 cells. J Agric Food Chem 58:12182-12191.
46. Miyoshi Y, Okada S, Uchimura T, Satoh E. 2006. A mucus adhesion promoting protein, MapA, mediates the adhesion of Lactobacillus reuteri to Caco-2 human intestinal epithelial cells. Bioscience, Biotechnology, and Biochemistry 70:1622-1628.
47. Ramiah K, van Reenen C A, Dicks L M T. 2007. Expression of the mucus adhesion genes Mub and MapA, adhesion-like factor EF-Tu and bacteriocin gene plaA of Lactobacillus plantarum 423, monitored with real-time PCR. International Journal of Food Microbiology 116:405-409.
48. Sahay B, Ge Y, Colliou N, Zadeh M, Weiner C, Mila A, Owen J L, Mohamadzadeh M. 2015. Advancing the use of Lactobacillus acidophilus surface layer protein A for the treatment of intestinal disorders in humans. Gut Microbes 6:392-397.
49. Juge N. 2012. Microbial adhesins to gastrointestinal mucus. Trends in Microbiology 20:30-39.
50. Milohanic E, Jonquières R, Cossart P, Berche P, Gaillard J-L. 2001. The autolysin Ami contributes to the adhesion of Listeria monocytogenes to eukaryotic cells via its cell wall anchor. Molecular Microbiology 39:1212-1224.
51. Ventura M, Jankovic I, Walker D C, Pridmore R D, Zink R. 2002. Identification and characterization of novel surface proteins in Lactobacillus johnsonii and Lactobacillus gasseri. Appl Environ Microbiol 68:6172-6181.
52. Hevia A, Martínez N, Ladero V, Alvarez M A, Margolles A, Sánchez B. 2013. An extracellular Serine/Threonine-rich protein from Lactobacillus plantarum NCIMB 8826 is a novel aggregation-promoting factor with affinity to mucin. Appl Environ Microbiol 79:6059-6066.
53. Veljović K, Popović N, Miljković M, Tolinački M, Terzić-Vidojević A, Kojić M. 2017. Novel aggregation promoting factor AggE contributes to the probiotic properties of Enterococcus faecium BGGO9-28. Front Microbiol 8.
54. Mulligan J H, Snell E E. 1977. Transport and metabolism of vitamin B6 in lactic acid bacteria. J Biol Chem 252:835-839.
55. Ito T, Yamamoto K, Hori R, Yamauchi A, Downs D M, Hemmi H, Yoshimura T. 2019. Conserved pyridoxal 5′-phosphate-binding protein YggS impacts amino acid metabolism through pyridoxine 5′-phosphate in Escherichia coli. Appl Environ Microbiol 85.
56. Walter J, Britton R A, Roos S. 2011. Host-microbial symbiosis in the vertebrate gastrointestinal tract and the Lactobacillus reuteri paradigm. PNAS 108:4645-4652.
57. Pijkeren J-P van, Canchaya C, Ryan K A, Li Y, Claesson M J, Sheil B, Steidler L, O'Mahony L, Fitzgerald G F, Sinderen D van, O'Toole P W. 2006. Comparative and functional analysis of sortase-dependent proteins in the predicted secretome of Lactobacillus salivarius UCC118. Appl Environ Microbiol 72:4143-4153.
58. Gagnon M, Zihler Berner A, Chervet N, Chassard C, Lacroix C. 2013. Comparison of the Caco-2, HT-29 and the mucus-secreting HT29-MTX intestinal cell models to investigate Salmonella adhesion and invasion. J Microbiol Methods 94:274-279.
59. Llanos-Chea A, Citorik R J, Nickerson K P, Ingano L, Serena G, Senger S, Lu T K, Fasano A, Faherty C S. 2019. Bacteriophage therapy testing against Shigella flexneri in a novel human intestinal organoid-derived infection model. J Pediatr Gastroenterol Nutr 68:509-516.
60. Nickerson K P, Senger S, Zhang Y, Lima R, Patel S, Ingano L, Flavahan W A, Kumar D K V, Fraser C M, Faherty C S, Sztein M B, Fiorentino M, Fasano A. 2018. Salmonella Typhi colonization provokes extensive transcriptional changes aimed at evading host mucosal immune defense during early infection of human intestinal tissue. EBioMedicine 31:92-109.
61. VanDussen K L, Marinshaw J M, Shaikh N, Miyoshi H, Moon C, Tarr P I, Ciorba M A, Stappenbeck T S. 2015. Development of an enhanced human gastrointestinal epithelial culture system to facilitate patient-based assays. Gut 64:911-920.
62. Senger S, Ingano L, Freire R, Anselmo A, Zhu W, Sadreyev R, Walker W A, Fasano A. 2018. Human fetal-derived enterospheres provide insights on intestinal development and a novel model to study necrotizing enterocolitis (NEC). Cellular and Molecular Gastroenterology and Hepatology 5:549-568.
63. Alifano P, Palumbo C, Pasanisi D, Talà A. 2015. Rifampicin-resistance, rpoB polymorphism and RNA polymerase genetic engineering. Journal of Biotechnology 202:60-77.
64. Bermudez-Brito M, Plaza-Díaz J, Fontana L, Muńoz-Quezada S, Gil A. 2013. In vitro cell and tissue models for studying host-microbe interactions: a review. British Journal of Nutrition 109:S27-S34.
65. Martínez-Maqueda D, Miralles B, Recio I. 2015. HT29 Cell Line, p. 113-124. In Verhoeckx, K, Cotter, P, López-Expósito, I, Kleiveland, C, Lea, T, Mackie, A, Requena, T, Swiatecka, D, Wichers, H (eds.), The Impact of Food Bioactives on Health: in vitro and ex vivo models. Springer International Publishing, Cham.
66. Altay G, Larrañaga E, Tosi S, Barriga F M, Batlle E, Fernández-Majada V, Martínez E. 2019. Self-organized intestinal epithelial monolayers in crypt and villus-like domains show effective barrier function. Scientific Reports 9(1):10140.
67. Yoo J-H, Donowitz M. 2019. Intestinal enteroids/organoids: A novel platform for drug discovery in inflammatory bowel diseases. World J Gastroenterol 25:4125-4147.
68. Walter J, Chagnaud P, Tannock G W, Loach D M, Bello F D, Jenkinson H F, Hammes W P, Hertel C. 2005. A high-molecular-mass surface protein (Lsp) and Methionine Sulfoxide Reductase B (MsrB) contribute to the ecological performance of Lactobacillus reuteri in the murine gut. Appl Environ Microbiol 71:979-986.
69. Maroncle N, Balestrino D, Rich C, Forestier C. 2002. Identification of Klebsiella pneumoniae genes involved in intestinal colonization and adhesion using signature-tagged mutagenesis. Infect Immun 70:4729-4734.
70. do Carmo F L R, Rabah H, De Oliveira Carvalho R D, Gaucher F, Cordeiro B F, da Silva S H, Le Loir Y, Azevedo V, Jan G. 2018. Extractable bacterial surface proteins in probiotic-host interaction. Front Microbiol 9.
71. Mayr U B, Walcher P, Azimpour C, Riedmann E, Haller C, Lubitz W. 2005. Bacterial ghosts as antigen delivery vehicles. Adv Drug Deliv Rev 57:1381-1391.
72. Doron S, Snydman D R. 2015. Risk and safety of probiotics. Clin Infect Dis 60:5129-S134.
73. Cristofori F, Dargenio V N, Dargenio C, Miniello V L, Barone M, Francavilla R. 2021. Anti-inflammatory and immunomodulatory effects of probiotics in gut inflammation: A door to the body. Front Immunol 12.
74. Fu X, Xu J-G. 2000. Development of a chromosome-plasmid balanced lethal system for Lactobacillus acidophilus with thyA gene as selective marker. Microbiology and Immunology 44:551-556.
75. Sambrook J, Russell D W. 2006. Transformation of E. coli by Electroporation. CSH Protoc 2006.
76. Holo H, Nes I F. 1989. High-frequency transformation, by electroporation, of Lactococcus lactis subsp. cremoris grown with glycine in osmotically stabilized media. Appl Environ Microbiol 55:3119-3123.
77. Pasch C A, Favreau P F, Yueh A E, Babiarz C P, Gillette A A, Sharick J T, Karim M R, Nickel K P, DeZeeuw A K, Sprackling C M, Emmerich P B, DeStefanis R A, Pitera R T, Payne S N, Korkos D P, Clipson L, Walsh C M, Miller D, Carchman E H, Burkard M E, Lemmon K K, Matkowskyj K A, Newton M A, Ong I M, Bassetti M F, Kimple R J, Skala M C, Deming D A. 2019. Patient-derived cancer organoid cultures to predict sensitivity to chemotherapy and radiation. Clin Cancer Res 25:5376-5387.
78. Saulnier D M, Santos F, Roos S, Mistretta T-A, Spinler J K, Molenaar D, Teusink B, Versalovic J. 2011. Exploring metabolic pathway reconstruction and genome-wide expression profiling in Lactobacillus reuteri to define functional probiotic features. PLoS One 6.
79. Blum, M., Chang, H. Y., Chuguransky, S., Grego, T., Kandasaamy, S., Mitchell, A., . . . & Finn, R. D. 2021. The InterPro protein families and domains database: 20 years on. Nucleic Acids Research, 49(D1), D344-D354.
80. Almagro Armenteros J J, Tsirigos K D, Sønderby C K, Petersen T N, Winther O, Brunak S, von Heijne G, Nielsen H. 2019. SignalP 5.0 improves signal peptide predictions using deep neural networks. 4. Nature Biotechnology 37:420-423.
81. Riedel C U, Monk I R, Casey P G, Morrissey D, O'Sullivan G C, Tangney M, Hill C, Gahan C G M. 2007. Improved luciferase tagging system for Listeria monocytogenes allows real-time monitoring in vivo and in vitro. Appl Environ Microbiol 73:3091-3094.
82. Oh J-H, Lin X B, Zhang S, Tollenaar S L, Özçam M, Dunphy C, Walter J, Pijkeren J-P van. 2019. Prophages in Lactobacillus reuteri are associated with fitness trade-offs but can increase competitiveness in the gut ecosystem. Appl Environ Microbiol 86.
83. Gibson, D. G., Young, L., Chuang, R. Y., Venter, J. C., Hutchison, C. A., & Smith, H. O. (2009). Enzymatic assembly of DNA molecules up to several hundred kilobases. Nature methods, 6(5), 343-345.
84. Alonso R, Mateo E, Churruca E, Martinez I, Girbau C, Fernández-Astorga A. 2005. MAMA-PCR assay for the detection of point mutations associated with high-level erythromycin resistance in Campylobacter jejuni and Campylobacter coli strains. J Microbiol Methods 63:99-103.
85. Cha R S, Zarbl H, Keohavong P, Thilly W G. 1992. Mismatch amplification mutation assay (MAMA): application to the c-H-ras gene. PCR Methods Appl 2:14-20.
86. Junop M S, Yang W, Funchain P, Clendenin W, Miller J H. 2003. In vitro and in vivo studies of MutS, MutL and MutH mutants: correlation of mismatch repair and DNA recombination. DNA Repair 2:387-405.
87. Zhang J, Kobert K, Flouri T, Stamatakis A. 2014. PEAR: a fast and accurate Illumina Paired-End reAd mergeR. Bioinformatics 30:614-620.
88. Kim E B, Son J S, Zhang Q K, Lee N K, Kim S H, Choi J H, Kang S K, Choi Y J. 2010. Generation and characterization of thymidine/D-alanine auxotrophic recombinant Lactococcus lactis subsp. lactis IL1403 expressing BmpB. Curr Microbiol 61:29-36.
89. Qiang Y Z, Qin T, Fu W, Cheng W P, Li Y S, Yi G. 2002. Use of a rapid mismatch PCR method to detect gyrA and parC mutations in ciprofloxacin-resistant clinical isolates of Escherichia coli. J Antimicrob Chemother 49:549-552.
90. Jouihan, H. 2012. Measurement of liver triglyceride content. Bio-protocol, 2(13), e223.
91. Leenhouts K, Buist G, Bolhuis A, ten Berge A, Kiel J, Mierau I, Dabrowska M, Venema G, Kok J. 1996. A general system for generating unlabelled gene replacements in bacterial chromosomes. Mol Gen Genet 253:217-224.
92. Wegmann U, O'Connell-Motherway M, Zomer A, Buist G, Shearman C, Canchaya C, Ventura M, Goesmann A, Gasson M J, Kuipers O P, Sinderen D van, Kok J. 2007. Complete genome sequence of the prototype lactic acid bacterium Lactococcus lactis subsp. cremoris MG1363. Journal of Bacteriology 189:3256-3270.

SEQUENCES

SEQ ID NO: 1
>srtA (LAR_0227)-Coding Sequence
5′/GTGAAAAAAGATAAAAAGCGATCATTTGAATGGTTACGGTGGACAGCGGTTGTTGTACTGTTACTGG
TATCAGTTGTCTTAATTTTTAACCAACAGATTAAGTCTTACTTAGTAGGGAGTTATAAACCTGAGATTACT
CGGCAAACGGTTCAAAGCAACCAAAAGAAAAAAGCAACCTATGATTTTCAAAGTGTCAAAGATCTTAAC
TTGCAAACAGCTGCCAAGGCTCGTGCAAATAAGCAATCGATTAATACCATTGGAGCAATTACGGTTCCG
GCTATTAATATGACGATTCCAATTGCTAATGGAGTTGATAATACAACTCTTGCGTTAGCAGCAGGAACCC
TTCGTCCAGACATGAAGATGGGGGAAGGCAACTATGCGTTAGCTGGTCATAATATGGCCCATGGGAGC
AAAATCCTCTTTTCTCCATTGTACTATCATGCTAAGGTAGGGCAGATGATTTATATCACCAACATGGATCG
CGTTTATGAATATAAGATTTATCAACGTGAATTCATTGCGGCAACCCGGGTTGATGTGGTAGACAATACG
CCGGAAAAGATTATAACTTTGATTACTTGTGACGCTACCGGGGCCAATCGGTTGATGATCCGCGGTAAA
TTTGTTAAATCAGAGCCATTTACGAAAGCACCACAAAATGTGCAAAAGAATTTTAGCGAAAAATATACG
ACAGGTCGTTAA/3′
SEQ ID NO: 2
>srtA (LAR_0227)-Protein Sequence
VKKDKKRSFEWLRWTAVVVLLLVSVVLIFNQQIKSYLVGSYKPEITRQTVQSNQKKKATYDFQSVKDLNLQTA
AKARANKQSINTIGAITVPAINMTIPIANGVDNTTLALAAGTLRPDMKMGEGNYALAGHNMAHGSKILFSPL
YYHAKVGQMIYITNMDRVYEYKIYQREFIAATRVDVVDNTPEKIITLITCDATGANRLMIRGKFVKSEPFTKAP
QNVQKNFSEKYTTGR
SEQ ID NO: 3
>cmbA (LAR_0958)-Coding Sequence
5′/ATGCTATCAAGAAAAAATTATAAGGAAACTATACGAAAACAGACACCTACAAAACAGTACTATACTA
TTAAGAAATTAACTGTTGGGGTTACTTCGGTATTAATTGGTCTATCCTTTATGGGAGAACTAGAAGGGGA
TAGCGTTCATGCGGACACGATGACAGCAAGCAGTGAGTCAACAAGTGTTACGTCGACGACTGCTCAGG
ATGGTTTAAAAAAATCTCCACAACTCTATTTGCAAGTTACTGATACAAATAACCCAAGTACACCATTAAGT
GCTTCATCCACAGGGACTAGTAAGAATGTTACCTCATCAGCTGCGGTACAAGTGAAGTCCGCTAGTGAT
GAAGAAGATAGTGATTCTACACTAGCTAAGGGAGAAAATAAATTTGCTCGGTCAGCAGTAAAAGATTCA
GTCACTGATGGGAAAACAAGTACAGCAGAAATTAATCCGGCAAAATTAAGCAGTCCTGCTTTAATAACG
CAACTCAACCAATCCTTAGCTAAGAGCAGTACGAGTGATGCAGCAAAAGCTAATGATGAGTTAGAAATT
AAAGCAACAGATCCGACTAATTATCCAAACTGTGGCGATGTGTATGGGCCATTATTTGAATTGGATGCTA
GCGGACAGCTTGTTAATAAAGATGAAGTTATATCTCTTAAAGATATGTATATTTTCCAAATATTGAAATTA
GTAAATACAAAAGATAGTGACTTTCAATATGTAATATTAACAATGAATCGTAAAGATACTGCAGATAGGT
CTGTATATCTTTTTGTAACTGGAAGCAATTATAGTAATGCTGTTGTTGTTAAAGTAAAGCCAAATGATACT
TATGAATTAAGTAAAACTGGATATAGTGTTACTTATACAGAACCAACAACTATAAATGGACATTATGTTG
ATGGAACTTTTTATGTTACAGGAAGTACTTACGATGATGGTTTTATAATGCCAGATTGGCAACTGCAGCA
CCTTCAGATTATATATAGTTTAGGAAATTATGATCCAAGCAATACTGACGCAACATCAGTTTGTGAAATA
ATGCCAAGTTATGAAAAGGTACCGGTAATTAAATATAGTGGAGTACCTTCAAATATTAGCCAACCTAAG
GTTTACATTACCGGGTTTACGGGTCAAGAGTTTAACGTTACAGATATTATTAACAATTATAAGAAAGTTT
TTAAGGGCTACTATCTTCAAAATCCTAATGTGGCGTCCATGGGAACTCTTTCCCAATTTGAGAATGGTGG
TTATTACTTAAAGACATATTATGATAATGATGGTAATGTTGACTTTAAGGGCTTGTATCATCAAATTGATG
ATCAGGGAACAATGAGTGTGAGTGTTCTTAATGCAGATAATAAAACAATTGTTGGACCTGAAAATATTC
TTGCTGGTAAATCGCATAACTTTAACTTTAATGGTCATAACTGGATTGCGCGGAATCCTTATGTCACTAGT
TCAGCTCACGAAGTCATATTAAAGTATGCTAAGTTAGGTTCAGTTATTCCTGTTGATGAAAACGGAAATA
AAATAAACGATGGATGGCAATATGTTAATGATCCAGATGATGCTTCCAAAGCCACTAGCCCATATGAAA
AAGCGCCAGTTATCGATGGTTATGTAGCTGTAAATCCAGATGAAACGATCGTTCTTCCTCATAACTTAAG
TAGTGACACAAAGATTTATTACCGAAAGAGGATTAAAGTTACCTATAGTGGTAGTGACAGCAAGACCTA
CGATGGTAACCCAGCTAACTTCGAGCCAACGACAGTTCAGTGGAGTGGCTTGAAAGGACTGAACACTTC
AACCTTAACGTCCGCTGACTTCACGTGGAATACTGCGGATAAGAAGGCACCAACGGATGCCGGTAAGTA
CACACTTAGTTTGAATACGACCGGAGAAGCAGCCTTACGTAAGGCTAACCCGAACTATGATCTCAAGAC
AATTAGCGGTAGTTACACCTACACGATTAATCCACTAGGGATTGATAAAGTTACCTATAGTGGTAGTGAC
AGCAAGACCTACGATGGTAACCCAGCTAACTTCGAGCCAACGACAGTTCAGTGGAGTGGCTTGAAAGG
ACTGAACACTTCAACCTTAACGTCCGCTGACTTCACGTGGAATACTGCGGATAAGAAGGCACCAACGGA
TGCCGGTAAGTACACACTTAGTTTGAATACGACCGGAGAAGCAGCCTTACGTAAGGCTAACCCGAACTA
TGATCTCAAGACAATTAGCGGTAGTTACACCTACACGATTAATCCACTAGGGATTGATAAAGTTACCTAT
AGTGGTAGTGACAGCAAGACCTACGATGGTAACCCAGCTAACTTCGAGCCAACGACAGTTCAGTGGAG
TGGCTTGAAAGGACTGAACACTTCAACCTTAACGTCCGCTGACTTCACGTGGAATACTGCGGATAAGAA
GGCACCAACGGATGCCGGTAAGTACACACTTAGTTTGAATACGACCGGAGAAGCAGCCTTACGTAAGG
CTAACCCGAACTATGATCTCAAGACAATTAGCGGTAGTTACACCTACACGATTAATCCACTAGGGATTGA
TAAAGTTACCTATAGTGGTAGTGACAGCAAGACCTACGATGGTAACCCAGCTAACTTCGAGCCAACGAC
AGTTCAGTGGAGTGGCTTGAAAGGACTGAACACTTCAACCTTAACGTCCGCTGACTTCACGTGGAATAC
TGCGGATAAGAAGGCACCAACGGATGCCGGTAAGTACACACTTAGTTTGAATACGACCGGAGAAGCAG
CCTTACGTAAGGCTAACCCGAACTATGATCTCAAGACAATTAGCGGTAGTTACACCTACACGATTAATCC
ACTAGGGATTGATAAAGTTACCTATAGTGGTAGTGACAGCAAGACCTACGATGGTAACCCAGCTAACTT
CGAGCCAACGACAGTTCAGTGGAGTGGCTTGAAAGGACTGAACACTTCAACCTTAACGTCCGCTGACTT
CACGTGGAATACTGCGGATAAGAAGGCACCAACGGATGCCGGTAAGTACACACTTAGTTTGAATACGAC
CGGAGAAGCAGCCTTACGTAAGGCTAACCCGAACTATGATCTCAAGACAATTAGCGGTAGTTACACCTA
CACGATTAATCCACTAGGGATTGTGACTGTAAATTACAAGGGCTATGATAAGAAAGTCTATGATGGTCA
ACCTGGAACGATTAATCCGGGTAAATTAACGTGGAGTAAGTTGCCAGATGGTACTTCATTGAAGATGCC
AACATGGAGTATAGATGATTTCGCTTGGGAAACAGCTGATGGCTTAGCACCAACGGCAGTAGGAACTTA
TCGGATTATCTTGACGGATGCTGGTAAGGCTGCACTAAAGAAGATTAATCCAAATTATGACTTAAGCAG
TATTACTGGTGTCTTTACTTATGAAATTAAGCCAGCACAGACACCAGAAATCTTAGGCCAAACACCTGAG
CAACAACCAGGCCAAAATACTAATCAATCAGGAGCTGAAAACGGCTTTGGTTCTTCTACAAGGCCTAAT
GCATCAACTAACTCCAATCTTAATCAACTTCCACAGACTGGTAATGAGCATTCTAATACTGCACTTGCTGG
TCTAGCATTGGCTTTCTTGACTGCTATGCTTGGTTTGGGCAAGAAGCGTAAACATGATTAG/3′
SEQ ID NO: 4
>cmbA (LAR_0958)-Protein Sequence
MLSRKNYKETIRKQTPTKQYYTIKKLTVGVTSVLIGLSFMGELEGDSVHADTMTASSESTSVTSTTAQDGLKKS
PQLYLQVTDTNNPSTPLSASSTGTSKNVTSSAAVQVKSASDEEDSDSTLAKGENKFARSAVKDSVTDGKTSTA
EINPAKLSSPALITQLNQSLAKSSTSDAAKANDELEIKATDPTNYPNCGDVYGPLFELDASGQLVNKDEVISLK
D
MYIFQILKLVNTKDSDFQYVILTMNRKDTADRSVYLFVTGSNYSNAVVVKVKPNDTYELSKTGYSVTYTEPTTI
NGHYVDGTFYVTGSTYDDGFIMPDWQLQHLQIIYSLGNYDPSNTDATSVCEIMPSYEKVPVIKYSGVPSNISQ
PKVYITGFTGQEFNVTDIINNYKKVFKGYYLQNPNVASMGTLSQFENGGYYLKTYYDNDGNVDFKGLYHQID
DQGTMSVSVLNADNKTIVGPENILAGKSHNFNFNGHNWIARNPYVTSSAHEVILKYAKLGSVIPVDENGNKI
NDGWQYVNDPDDASKATSPYEKAPVIDGYVAVNPDETIVLPHNLSSDTKIYYRKRIKVTYSGSDSKTYDGNPA
NFEPTTVQWSGLKGLNTSTLTSADFTWNTADKKAPTDAGKYTLSLNTTGEAALRKANPNYDLKTISGSYTYTI
NPLGIDKVTYSGSDSKTYDGNPANFEPTTVQWSGLKGLNTSTLTSADFTWNTADKKAPTDAGKYTLSLNTTG
EAALRKANPNYDLKTISGSYTYTINPLGIDKVTYSGSDSKTYDGNPANFEPTTVQWSGLKGLNTSTLTSADFT
WNTADKKAPTDAGKYTLSLNTTGEAALRKANPNYDLKTISGSYTYTINPLGIDKVTYSGSDSKTYDGNPANFE
PTTVQWSGLKGLNTSTLTSADFTWNTADKKAPTDAGKYTLSLNTTGEAALRKANPNYDLKTISGSYTYTINPL
GIDKVTYSGSDSKTYDGNPANFEPTTVQWSGLKGLNTSTLTSADFTWNTADKKAPTDAGKYTLSLNTTGEAA
LRKANPNYDLKTISGSYTYTINPLGIVTVNYKGYDKKVYDGQPGTINPGKLTWSKLPDGTSLKMPTWSIDDFA
WETADGLAPTAVGTYRIILTDAGKAALKKINPNYDLSSITGVFTYEIKPAQTPEILGQTPEQQPGQNTNQSGAE
NGFGSSTRPNASTNSNLNQLPQTGNEHSNTALAGLALAFLTAMLGLGKKRKHD
SEQ ID NO: 5
>11993 (LAR_0044)-Coding Sequence
5′/ATGAGAAATTCGAATACAAATAATTGGCGTTCAACTGCTTTGATTGCGGGGGCAATCTTGACAGCAG
GTGTCGTTACCACCACTTCAACGACAGATGTACACGCTGATAATAGTCAAAATAATGCACAAGTAAGTAA
TGACCAAGTTACTTACGACCAAGTTCGGGCAACTGCTGATCAGCAACTTGCACAATTGCAATCAGCTAAT
GATAGTAAAGAAGCTCAACAAGCTACGGCCAACGAGCAATCAAATGCTGCGGATCTTGCACAAATCAAC
GCACAAATTGATCAATTAAAAGCTAGTCATGCTGCTCTAGAACAAGAACAAGCCGGTGCTATTGCACAA
GCACAAGCTTCTACTGCTGCTTCTATTGAAGCACAAACTTCAGCTGCTAATGCTGAATACCAACAACAAA
TTAACGCACAACAACAAAACGAAGCCACTCAACGTGCAGCAAATGATCAACAGTACGCACAAGCTGTTA
GTCAAGCTGCTGAAAATCAACAAGCTATTGCTGATATCAATGCACAATATCAACAAGCTTTAAGTGATGC
AGCTAATACGCAGACAAGCCAAGCACAGGCTGCTGTCAATAATTACAACTCAGCTGTTGCTAAGGAAAA
TGATGCTTACCAAGGTAAGCTATCTGCTCAACAAGCAACTAATGCAGAAAATGTTAAAAATGCTGAAGC
ATCTCTTGCAACCGCAACTGCTAATGCTAACAAGCCTTATGAAGTTGCAACTACTAACGATGTTGCTGTT
ACTGGCCGAGTTGCTAAACCACAAACAGATGTTAAGGTTCCTGACTATGTAGCAAAATACGGAATTCAT
CTAGAAGTTCGTGGCTATAATGGTGCTATTCCTGTATCTGATTATACTTATCCTGAATTTGCTAATACTTA
CTATGGTGAATATCCTGATAGTGCAGTAATTACAAATAAGGCTGCTGACTCAACTCCACATGATATTTCA
GAGATGCCATTAGATTTCCAACCAGGTCTATTAAGTTACGATACTAAAAATGATCATTCAGAAAAAGTTT
CTGCTGATGGATTAACAGCTGCCCAAGTTCAAGTATTACGGGCATTAGCTCTTTCATGGGAAAATGGATT
CCGTAACAATGTTTTCCAAAACTACCGTGAATTTTACAATGCGGTTAATAAGAATGATGGCTTTGTGAAC
GTTGCTCCAGTTGATTTAGTAAGCACTGACTTTGCTGATAACATTGCTAACCAAGTTGTTGCTAACCGGA
CAAAGTACAATGTTGATAATAATAGTCATACTGTTATCAGCAGTGGAATGCCTACTGAAGCAACTTATAG
TAGCATTATTAATAATGCCACCCAAAACTTGCAAAAGAGTTTAACAAGTGAATTTAATGGTAAGACAGTT
TACACTGACGTTAATGAAAACTTAACTACTATGCAAGCTGGGACGCCAACTCTTCTAAATTACGCTATTA
ATCTCTACAACTCAATGCAGGGTATGTACTATGGTGAATTGGTAAACCCAACACACATCGGTGGACATG
CAGTTAATCTTCTTCGTGCTGGTGTTCGCACTGTGGGAATTGGCTTCCAAAAGCTAACTGATGAAATGAC
TGCTAAGAATGGTTCAGGTGTTTCTGATCGCAAGAACCCATCATATGCTGTAACATTTGATCATGTCGGA
TTTAACATTGATCACGATGTTAACTTAACTAAAGAAGTTCAAAATCGTAATGTATGGGGAACTTCAACTG
TAGATGCACAATTAAATGCTATTAAGACTGCTCAATATCATGTTAATAAAGTACCGGGGACAAAGACAG
TTACGCCAACTGCTACTGATGTGCAAAAAGCTACTGCTAGCGAACAACAAAAATTAGCTAATGTTAAGG
CACAAGCACAACAATCTCTTGATGCATTAGCTAGTGCCCACCAAGCTAACTTAAATAAGCTTGAACAACA
ATATAATGATGCGAAGGCTCAAGTAAAAGCTAACTATGATACTGCAGTTACTAAGGCAGCTAGTGTTCG
TGATGCTGCTCTTAAGGCTACTGGAGCTGTTGACCTTCAAGCTTACAAGGCTCAATTAGACAGTGCCTAC
CAAAACTTAGTAAAGGCTGATCAAGTAGCCGCACAAAAGCTTGCTGCTGATCGTGACGCTAAGATCGCT
AATATTAAGAAAACTGAAAATGCAAAGCTTGATGCTAAGATTAATGAACTTGTTCCAAGCATTGACCCAC
AAATCAAGCAATTGCAAGATCAACACCAAGCAGTTATTACCCATGGTGATGCTGTTCTTGCTGAATTAAA
GGCTGAAAATAAGGCTGCTTATAACAAACTTGCTGAACAATTCAACGTTCAATTAGCAGCTATTCAACAA
CGTGAAGATGCTAAGAGAGCTAATGAAGTTCGGTTAGCAAATAATACTGTTGTTCTTCCTACAGCAGTG
AAAGCAGCTAATGATGGCAATGTTGATACTGTTGCTTTCCCAACGGTTACAAACCGTCTACAAGCTGAAA
CTACTGGACGCGTTCAACCAGTTTCAGCACCATCGATTGTAAAGACAGCTACGCAAACGCCAATTGCAGT
TGCACCAGTTGTAACACAAACAGCTGCAGTTGCTGCTACTACAAATAATGGTACACAAACACGGGTTGC
TTCTACTGCTGATAATAAAGCCACTAAAGCAGACAACAATGAAAAATCAGTTGTAAAAGCTGACACTGTT
GCTAAGAAGCAAACTCCTGCTAAGAAGAGTGAATCATCACAAAAGGTTGATCAATCTTCTATGTCAATTG
TTGCTTTAGCTGCCACTGCATTACTTGGTACACTCGGTATCACTTACAGCTCAAAGAAGCGCCACAACTA
A/3′
SEQ ID NO: 6
>11993 (LAR_0044)-Protein Sequence
MRNSNTNNWRSTALIAGAILTAGVVTTTSTTDVHADNSQNNAQVSNDQVTYDQVRATADQQLAQLQSA
NDSKEAQQATANEQSNAADLAQINAQIDQLKASHAALEQEQAGAIAQAQASTAASIEAQTSAANAEYQQQ
INAQQQNEATQRAANDQQYAQAVSQAAENQQAIADINAQYQQALSDAANTQTSQAQAAVNNYNSAVA
KENDAYQGKLSAQQATNAENVKNAEASLATATANANKPYEVATTNDVAVTGRVAKPQTDVKVPDYVAKY
GIHLEVRGYNGAIPVSDYTYPEFANTYYGEYPDSAVITNKAADSTPHDISEMPLDFQPGLLSYDTKNDHSEKVS
ADGLTAAQVQVLRALALSWENGFRNNVFQNYREFYNAVNKNDGFVNVAPVDLVSTDFADNIANQVVANR
TKYNVDNNSHTVISSGMPTEATYSSIINNATQNLQKSLTSEFNGKTVYTDVNENLTTMQAGTPTLLNYAINLY
NSMQGMYYGELVNPTHIGGHAVNLLRAGVRTVGIGFQKLTDEMTAKNGSGVSDRKNPSYAVTFDHVGENI
DHDVNLTKEVQNRNVWGTSTVDAQLNAIKTAQYHVNKVPGTKTVTPTATDVQKATASEQQKLANVKAQA
QQSLDALASAHQANLNKLEQQYNDAKAQVKANYDTAVTKAASVRDAALKATGAVDLQAYKAQLDSAYQN
LVKADQVAAQKLAADRDAKIANIKKTENAKLDAKINELVPSIDPQIKQLQDQHQAVITHGDAVLAELKAENK
AAYNKLAEQFNVQLAAIQQREDAKRANEVRLANNTVVLPTAVKAANDGNVDTVAFPTVTNRLQAETTGRV
QPVSAPSIVKTATQTPIAVAPVVTQTAAVAATTNNGTQTRVASTADNKATKADNNEKSVVKADTVAKKQTP
AKKSESSQKVDQSSMSIVALAATALLGTLGITYSSKKRHN
SEQ ID NO: 7
>cidi (LAR_0983)-Coding Sequence
5′/ATGAAGAATAATAGTTCAAAATATTGTTTATTGTTAGGGACAGCATTGTTAGGATTATATTTCCAAGC
TAATAGTGTTCATGCGGATATGACTGGGACAACAGTTAATGGTGAAACTGCCCATAGCAATGTTACGCC
AATGGTCCAGACTAATAAGGATGAAGCAAGTACTCCGCAAACGACTACTGATTGGTCTGACCTAGCCAA
ATATCAAAATGACATTCCAGTTCAGATTTTAGGAATCAATGACCTGCATGGTGGGTTAGAAACCACTGG
ATCAGCTACGATTGGAGATAAGACGTATTCGAATGCCGGAACAGTTGCACGCCTAGCTGGTAACCTTGA
TGCGGCAGAGGAAAGTTTTAAGAACGCTAATCCGACAGGAACCTCAATTCGGGTAGAAGCCGGAGATA
TGGTTGGGGCTTCTCCAGCAAATTCTGCTCTTCTTCAAGACGAATCAACTATGCATGCTTTAGACGCAAT
GCATTTTGAAATAGGAACTTTGGGAAACCATGAGTTTGATGAAGGTTTAGCTGAGTATATGCGGATTGT
TAATGGTGGTGAACCTACTAAACAATATAATGAAGCTGAGATGGCCTATCCTCATGTGAAAACAGGGAT
TAATATCATTACTGCCAATGTTGTAAATAAATCTGATGGTCAAATCCCATTTGGAATGCAACCATACTTGA
TTAAAGAAATTCATACTAGTGATGGTAAAGTTGCTCGGATTGGCTTTATTGGGATTGAAATTACTTCCCT
ACCAATTTTAACCTTATACGATAATTACAAAGATTATGATGTTTTAGACGAGGCTGAAACAATTGCAAAA
TATGATCAAATTTTACGTAAAAAGGGTGTTAATGCAATTGTAGTTCTTGCCCATACAGGGGTTTCAACGG
ATAAAGATGGCAGCACTAAAGGTAATGCTGTTGATATCATTAAGAAGCTTTACCAAATTGACCCTGATAA
TTCTGTCGACCTTTATGTTGCCGGTCACTCCCACCAATATGCTAATGCTACTGTTGGTAGTGTAAAATTAG
TGCAAGCCATTTACACAGGTAAGGCTTACGATGATATTATCGGTTACATCGATCCAACAACTAATGATTT
TGCACCGAATAGTCTCGTTTCGCATGTCTTTCCTGTATTATCAGAAAAGGATGCGCCTAATATCAAAACA
GATGCAAATGTTACAGCAATTGTTGAAGATGCTAACAATAGAGTAGCACCAATTATTAATAAGAAAATA
GGGGAAGCTGCTACCACAGGCGATATTCTTGGACGACTACATAATACCCCTACTCGTGAAAATGCCGTT
GGTGAATTAGTTGTCGATGGTCAATTATATGCCGCTCATAAGGTGGGCTTACCAGCTGATTTTGCGATGA
CTAATACAGGCGGTGTTCGTGCAGATCTGCATGTTAATCCTGATCGTTCCATTACATGGGGAAGTGCCCA
AGCGGTTCAACCATTTGGTAATATTTTGCGGGTAGTTGAAATGACAGGGGCACAAATAGTTGAAGCCTT
GAATCAACAATATGACGAAGATCAAGCTTACTACTTACAAATTTCTGGACTTCATTATACTTATACTGACC
AAAACGATCCTAACCAACCATATAAGGTCGTTCAAGTTTATGACCAACATAATCAACCGCTTGATATGAA
TAAGACTTACAATGTTGTTATTAATGACTTTTTAGCAGGTGGCGGAGATGGCTTTTCTGCATTTAAGGGT
ACCAAAGTTGTCGGGATTGTCGGGCAAGATACAGACGCGTTTATTGATTATATTACTGATATGACTAATG
ATGGTAAACCAATTACCGCACCAACAATGAACCGTAAGATTTACTTGACTGCTGAACAATTAGCGAAGG
CTGACGCAGATTCACAGTCACAAACAGGAACTAATCAGAACACTCAAAACGATGCTAATTCCCAGACTG
AAGGAAATCAGCTTCAAGAAGTTCCGAGCCAACCGGTATCTCCAACAGTAACCTTGCCAACAACAGCTG
GTCAACCCGCCGAAACTGTTACATTACATGCTCAATCTAAGCAACAAACCGTAGCTGCTAATAATCAATT
AATTAATTTGACGCCTACATCAATTAATGGCCAAAAACAAAAAGCAACTGACCAGCAAGCAGCTTTACCA
CAAACTGGTAACGATGAAGATCTTGCATTACTTCTTCTCGGAAATTCATTAATGGCAGCAACCGGATTGA
CAATTATTGATCGCAAGCGTAAACATGCTTAA/3′
SEQ ID NO: 8
>cidi (LAR_0983)-Protein Sequence
MKNNSSKYCLLLGTALLGLYFQANSVHADMTGTTVNGETAHSNVTPMVQTNKDEASTPQTTTDWSDLAKY
QNDIPVQILGINDLHGGLETTGSATIGDKTYSNAGTVARLAGNLDAAEESFKNANPTGTSIRVEAGDMVGAS
PANSALLQDESTMHALDAMHFEIGTLGNHEFDEGLAEYMRIVNGGEPTKQYNEAEMAYPHVKTGINIITAN
VVNKSDGQIPFGMQPYLIKEIHTSDGKVARIGFIGIEITSLPILTLYDNYKDYDVLDEAETIAKYDQILRKKGV
NAI
VVLAHTGVSTDKDGSTKGNAVDIIKKLYQIDPDNSVDLYVAGHSHQYANATVGSVKLVQAIYTGKAYDDIIGY
IDPTTNDFAPNSLVSHVFPVLSEKDAPNIKTDANVTAIVEDANNRVAPIINKKIGEAATTGDILGRLHNTPTRE
NAVGELVVDGQLYAAHKVGLPADFAMTNTGGVRADLHVNPDRSITWGSAQAVQPFGNILRVVEMTGAQI
VEALNQQYDEDQAYYLQISGLHYTYTDQNDPNQPYKVVQVYDQHNQPLDMNKTYNVVINDFLAGGGDGF
SAFKGTKVVGIVGQDTDAFIDYITDMTNDGKPITAPTMNRKIYLTAEQLAKADADSQSQTGTNQNTQNDAN
SQTEGNQLQEVPSQPVSPTVTLPTTAGQPAETVTLHAQSKQQTVAANNQLINLTPTSINGQKQKATDQQAA
LPQTGNDEDLALLLLGNSLMAATGLTIIDRKRKHA
SEQ ID NO: 9
>LAR_0903-Coding Sequence
5′/ATGATAGCTTTTTTCGATTTAAAAAATGCGAATTGCTTCTTTTTACGTATATATATTAAAGAGTATATA
AAAGAGGAGCGAGATAATATGAACCATTTAAACCACCTTCATGTATTGCGAAACACTTGCTTAGTAGGC
TTAACGACAGCGTCAACGATCCTTTCGCTGTCGTTAAATATTCCACCAGTTTTAGCTGCAACTCAAGAGCC
AATTACGCATCCCGTATCTAACACAAATAATCAGAAAGAACGTGTCCAAATCAAACGTGTAATTTATTTTC
ATCTCTTAAATGAAGTTAAAAAAGTGGAACAAGTGTCTTATGCTCACCGTGAAGTACCGAATGATTCAAC
CAAAAAGGCAAATTGGATTATTGAACCTTTTGAAGAAGTTAATATCCCTGAGCAAGCTGGTTTTAGACCA
TCAATGGATAAGATTCCAACTGTTACAGAAATAGAAGATCTATCGCAATTAAAAAATACGATAAATGTTT
ATTATCAGCCCCTCAATAAAGATGAAGGGCCAAAAAAGGACAGCAAAGAGAATACGTTAGCAGAAAAG
ACTAGGGGTGAGCAAGTAAAGGAGAATAAAAAAGAGGAAAAACAAGATTCGCAGGATAATTACCCCGT
GGTAGAAGATCTGGGGGATGATAATCAGGTCACGGAAAATTATGGGGAGGTAAATAATACAAGAAAG
CAGCAAAATTTGCATCAACGGGTAACAGGGATTAATAATCGACTAAATCGTTCACATCGGGACTCACGT
AAAAACGATAACTCTGACCAACAAATACTTCCACAGACGGGAAATGAGACCGATAATTTAACGACTTTTC
TTGGTTTGGGGATAACGGTAATGGTCGCGGGGATGAGTCTTTTTTCGTTAAAGAAAGCGCACAAAAATA
AATAA/3′
SEQ ID NO: 10
>LAR_0903-Protein Sequence
MIAFFDLKNANCFFLRIYIKEYIKEERDNMNHLNHLHVLRNTCLVGLTTASTILSLSLNIPPVLAATQEPITHP
VS
NTNNQKERVQIKRVIYFHLLNEVKKVEQVSYAHREVPNDSTKKANWIIEPFEEVNIPEQAGFRPSMDKIPTVT
EIEDLSQLKNTINVYYQPLNKDEGPKKDSKENTLAEKTRGEQVKENKKEEKQDSQDNYPVVEDLGDDNQVTE
NYGEVNNTRKQQNLHQRVTGINNRLNRSHRDSRKNDNSDQQILPQTGNETDNLTTFLGLGITVMVAGMSL
FSLKKAHKNK
SEQ ID NO: 11
>pilP (LAR_0989)-Coding Sequence
5′/ATGAAGAAAAGAAAATTAAAGAAGAGTTTAGCAACAACTGCGACGGTGATGGCTGTTACAACGGGG
GTGGCGGCCATTAGTAATTCCGCCAAAGCTGATACAGTGCAAAATAATAAAAATACTATTCAACAAACG
CTGCCTGATGCGAACCAACAAGCTCAGCAGAATGTCTGTGCAGCCCAAGACGCGGTTAATAAAGCCAAT
TTTGATGTAGCAACCGCAAATAATGATCTTAATATTGCTAATCAAAATTTGGCAGACGCGGATCAAAATG
TCGATTCTAATAAGAATCAAGTTAAACAAACGAAAGAGCAATTATCATCTCTTGAGCAAATGAAAGAAA
ATGCTCAACAAGTTTTAACTAACGCTCATGATGAATATTATGAATTATTGAAGCAACCAGTTGATGTTTCA
TACACGGCATTTTCTGGTGGTGGATTGAGCGTTTATGAAAGATCAATGCTTGATTATATTACTTATTATAA
CCAAGGAAACTTAACTTCTAAGAATCAAGCGTATAAAAATTTCCTTGCGAATGACTATCAAGGAAACATT
GATTTATTGAAAAAGATTATTGCTGAAAACGAGAGAACCATTAATTATCTGAAAATGGGTCGACCATTTG
AAGATAATATTAGAAATCTTGAGCAAGCAAGATTAACAATAAAAGAAGAACTAGAAGAATTATATAAGG
TAGTGGAAGCCCAAAAAGACGGGAAATATTATAAACCAAAAGATGATATTAGGTGGGCGAATATCGAT
TCTTATGGTTTGTCATATTCTCCAGTAATGTCTAATGGATGGTATGAGGATAATCCAACTGACCAAATAA
GTGACTTAATTGAAAATCACGAGGAAGCTCTAAACAAAATTCAACAAAGATTAGCCGAATTACATAATA
AAATGCAGTCGGCTGTAGAAAAGGCAGAAAAAACATCCATTGCTGCTCAAGAATTACTTCAAAAGTTAG
AGGAGATTTCACAACCGCTTATTACTGCTACTCAACAAATTAAAGATGCCGAAAGTGCAATGTCACAAGC
TCAACAAAATCTGACACTTCAAGAAGAACTACTTACTCAGGCCAAAGATAAGCGCAATGTGATGCAATCT
CAAGCAATCAATGCGCAAACCAAACTCTTGCAAGCACTTCTCAATAAAACAAATTCACAAGAAAAGTTAG
CGTCAGCAAAAGAGAAACTTCCGGCACAAACTGATACCTTAAAGTATAGTTCGCTAATCAACCTTGAACC
ACTAACGGTTGAGCCAGGAGTAACTCCTACTCCTAAAGTTACGACAGCGATTGCGGTTGAAAATGATGA
TCATCAGAATAAAGCTGTTATTACTTTGCCTGGCGATGAACAAGAAAATAAGCTGCCTCAGGGAACAAA
GGTAGTGTGGAAAGATGATGCTAAAGTTGCGACCGACCTTCAACGTCCCGGAAAGCATACTGAAGATG
TCTTAATTGTTTTTCCTGATGGATCGGTGATTTTAACTAGCGAGCAAGTTACTGTTACCGCGACAGAAAA
ACAGGCAGACGCTAATAAAGTGACTTCGCCTGTACAAGAAACGGTTGATAGTACCCAAGAAAATAATAT
GCGTGTGAACGATCTTTCTACCCAGGTAATAGAAGTTAATGATAATAATCAAACCTCAGCACCAGAAATA
TTGGTGACTCCGCAAACCAAGCTAAATACCGTCCAACCAAACGGAGTAATAAAAGGGCAGACAGCTCAG
AATAAGATTACCCAGCCTTCATTAGCTAAGTCTTTTGAGGATAAGACAAGAAATAATGTTTTGCCACGAA
CCGGAGATGAATCTTCACTTCCGATTATTGTCCTTGGCGCTGTTATTTGGCTTGCAGGAATTGGGGCAAC
ACTTAAACGGTACGAATAA/3′
SEQ ID NO: 12
>pilP (LAR_0989)-Protein Sequence
MKKRKLKKSLATTATVMAVTTGVAAISNSAKADTVQNNKNTIQQTLPDANQQAQQNVCAAQDAVNKANF
DVATANNDLNIANQNLADADQNVDSNKNQVKQTKEQLSSLEQMKENAQQVLTNAHDEYYELLKQPVDVS
YTAFSGGGLSVYERSMLDYITYYNQGNLTSKNQAYKNFLANDYQGNIDLLKKIIAENERTINYLKMGRPFEDNI
RNLEQARLTIKEELEELYKVVEAQKDGKYYKPKDDIRWANIDSYGLSYSPVMSNGWYEDNPTDQISDLIENHE
EALNKIQQRLAELHNKMQSAVEKAEKTSIAAQELLQKLEEISQPLITATQQIKDAESAMSQAQQNLTLQEELLT
QAKDKRNVMQSQAINAQTKLLQALLNKTNSQEKLASAKEKLPAQTDTLKYSSLINLEPLTVEPGVTPTPKVTT
AIAVENDDHQNKAVITLPGDEQENKLPQGTKVVWKDDAKVATDLQRPGKHTEDVLIVFPDGSVILTSEQVT
VTATEKQADANKVTSPVQETVDSTQENNMRVNDLSTQVIEVNDNNQTSAPEILVTPQTKLNTVQPNGVIKG
QTAQNKITQPSLAKSFEDKTRNNVLPRTGDESSLPIIVLGAVIWLAGIGATLKRYE
SEQ ID NO: 13
>fbpA (LAR_0878)-Coding Sequence
5′/ATGTCTTTTGACGGCTTGTTTACTCATGCAATTGTCCATGAGCTAGATCAAAAATTAACTACTGGTCG
GGTAGCCAAAGTTAGTCAACCTTACCCTGCAGAATTAATCATTACGATCCGTGCTCACCGGCACAACTAC
CCATTACTCATTTCTGCTAATCCAACATACCCACGAATCCAAATTACTGAGATCCCATACAAAAATCCGGC
GGTTCCAACTAATTTTACAATGACAATGCGGAAATACCTTGAAGGTGCAATCGTCAATAAAATTGAACAA
GTTGATAATGATCGAATTATCAAAATTACCTTTGACACTCGTGATGAATTAGGAGATAGTCAACAATTAG
TTCTTGTGAGTGAGATTATGGCCCGGCATAGTAATATTTCACTCGTTAATCTCAAGACGGGTAAAATCAT
TGACACCATTAAGCATGTTGGCTCAGATCAAAATCGGGTTCGCCTGTTATTACCTGGTGCTACCTTTGTCA
TGCCACCAAAGCAAGATAAAGTTAACCCTTACTTGCCAAACCAGGTCTACTCTGATCTTGTAAGACAAAC
CGATGACACAGTAGAATTAAGTCATCAGTTACAGGAGCATTATCAAGGATTTGGCAAAGACTCCGCACG
GGAATTAGCTGCAGAATTACTGCAAAGTGATAATTTACCAGCTACCTATCAGCACTTTCTTAAGCACTTT
GAAAATCCAGAGCCAGTATTAATCACCCACTCTAATGGAAAAACTCAATTTGCTGTGTTCCCTCCATTAAA
TATTGATGGTGAGTTGCAACATTTCGACTCCCTCTCTGCCCTTCTCGATGCCTTCTATGCTAATAAAGCAG
AGCAAGATCGTTCCAAAGAATTAGCTGGGCAAGTGTTAAAGGTATTGAAGAATGAGCTAAAAAAAGAT
CGGCGAAAAGTAAAAAAACTGCAACAACAATTACAAGATGCAGCAACTGCAGATCAGTACCGAATTTGT
GGTGAGATCCTTACTACCTATCTTAGTAAATTAACTCCAGGAATGAAAGAAATCGAACTTCCTAACTTTTA
TGATGATAATAAGCCACTAAAAATCAAGCTTGCTCCAGAATTATCACCATCGCGCAACGCCCAAAAGTAC
TTTACTAAGTATAATAAGCTCAAAACCTCTGTTGAGTATGTAAAGGAACAGCTAAAACTGACAAATGATG
AAATCAAATACTTTGAAAACATTGAAAACCAGATTAAATTAGCTGCTCCCGCTGATATTCAAGAGATCAA
ACTGGAACTGCAAGAACAGGGTTATATTAAAAAGAAGAAAAGCGGGAAAAAGCAACGAAAAGTTAAG
GTAAGTGCTCCAGAAGAATTCCATACTAGTGATGGCACTACGGTTTTAGTAGGTAAAAACAACCTGCAA
AATGACCGTCTTAGCTTTAAAATTGCTAATAAAAATGAAATTTGGTTACATGTTAAAGATATCCCAGGTT
CCCACGTGGTAATCCGCTCAACAAACCCATCTGAAGATACGATTCTAGAAGCCGCACAGTTAGCAGCAT
ACTTCTCTAAAGGTCGCGATTCAGATAATGTGCCCGTTGATTATCTTCCTGTCAAGCGACTTCATAAGCCA
AACGGAGCTAAACCTGGCTTTGTTATTTTTACAGGTCAGAAAACACTTTATGTAACACCGCATAAACTTTC
TAACTAA/3′
SEQ ID NO:14
>fbpA (LAR_0878)-Protein Sequence
MS FDGLFTHAIVHELDQKLTTGRVAKVSQPYPAELIITIRAHRHNYPLLISANPTYPRIQITEIPYKNPAVPTN
FT
MTMRKYLEGAIVNKIEQVDNDRIIKITFDTRDELGDSQQLVLVSEIMARHSNISLVNLKTGKIIDTIKHVGSDQ
NRVRLLLPGATFVMPPKQDKVNPYLPNQVYSDLVRQTDDTVELSHQLQEHYQGFGKDSARELAAELLQSDN
LPATYQHFLKHFENPEPVLITHSNGKTQFAVFPPLNIDGELQHFDSLSALLDAFYANKAEQDRSKELAGQVLK
VLKNELKKDRRKVKKLQQQLQDAATADQYRICGEILTTYLSKLTPGMKEIELPNFYDDNKPLKIKLAPELSPSR
N
AQKYFTKYNKLKTSVEYVKEQLKLTNDEIKYFENIENQIKLAAPADIQEIKLELQEQGYIKKKKSGKKQRKVKV
SA
PEEFHTSDGTTVLVGKNNLQNDRLSFKIANKNEIWLHVKDIPGSHVVIRSTNPSEDTILEAAQLAAYFSKGRDS
DNVPVDYLPVKRLHKPNGAKPGFVIFTGQKTLYVTPHKLSN
SEQ ID NO: 15
>autolysin (LAR_1284)-Coding Sequence
5′/GTGACTAATAAAAAGCATTATAAATTATATAAGTCGGGGAAAAATTGGTGTGTCATGGCAATTACTG
CACTAGCATTAACAGTTAGTTTAACTGGGGTTGCTAGTGCTGATACAACAGTTGATACAGCACAGGCAG
AAACGAGTTTAGCACAGAGTTCAGCATCTGATGCTACTCAAATAGAATCAACCGATGCTAAAATTGGCG
AAAGTGAGACTAATCAGTCTAATCAAAATAATCAACAGGGAAATATTAGTACTCAACAAGCCACGGATC
AAAAAGCAAACTCAGTAACACCAAAGTCAACAGAGATATCAACACCAGTAAAAGATGGTTGGGTACAA
GAATCTAATGGCTGGACTTTTTATAAAAATGGTAAAACTGATTTAGGTCGTACTTATTCATATTTACCAAC
AATTACAAGTAATGGTAAAGGCAGCGGAAGCAACTGGTACTTAACGGATAATGGTGTAATTCAGACAG
GTGTTCAAAAATGGGCCGATACCTATTACTATTTTGATCCAAGCACTTACCTTCGCGTGGATAATGACTA
CCGTCAATCCCAATGGGGCGATTGGTATATGTTTGGTAAAGATGGTAGAATTGCTACCAAAGTTTATCAA
TGGGCTAGTACGTACTATTATTTTGATCCAAGTACTTACCTTCGCGTGGATAATGATTACCGTCAATCCCA
ATGGGGTGACTGGTATCTCTTTGGCAATGATGGTCGTATTCAAACCGGAGTCCAAAAATGGTATGATAC
TTATTATTACTTTGATCCAAGTACTTACCTTCGAGTAGACAATGATTACCGTCAATCCCAATGGGGGGATT
GGTATATGTTTGGTCCAGATGGTCGAATTGTTTCAGGACTTTATGGCTGGAAGGAAAGTTTGTATTACTT
TACTCCTTACTTATATACCAAGGCAACTAACCAGTGGGTTTCAGCGAATGGTAAAAGTTATTGGGCAAGT
GGCAGTGGAATTATTACTTCTGGATTGAATTCAATTAACAACTATATTCTTAATAATAATCTTGGTCATGC
TAATATTACATTTTATGATAATGGGCAGGCCATTCCTTTGAATATTACTGGAAAATATAGTGGTACGGGT
AATGGATTACCAAATATAGTTATCGTCCATGAAACTGCTAATCCTAATGATAGTATTTGGGGTGAAATAA
ATTACGAAAAGAATCATTATAATGATGCATTCGTTCATGCCTTTGTAGATAATAACAATATAATTCAGATC
TCAAATACTGACCATGAGGCTTGGGGTGCAGGTTATCCTGCTAATGGTCGGGCCGTTCAATTTGAACAA
GTTGAGGTACATAATGCAGATGCGTTTGCTCGGGAACTTTCTAACGCTGCTTACTACACTGCATATATTA
TGCACAAGTATGGGTTTGCGCCTTCGTTAGTATCTAATGGTAACGGAACTCTATGGTCTCATCATAATGT
ATCTCAATATTTAGGTGGTACTGATCATACAGATCCGGATGGTTATTGGTACACAAATGCCCACAATTTC
TATGGTACTGATTATACAATGCGTGATTTTTATGAGTTGGTAAGCCTATATTATGGTGAATTCTAA/3′
SEQ ID NO: 16
>autolysin (LAR_1284)-Protein Sequence
VTNKKHYKLYKSGKNWCVMAITALALTVSLTGVASADTTVDTAQAETSLAQSSASDATQIESTDAKIGESETN
QSNQNNQQGNISTQQATDQKANSVTPKSTEISTPVKDGWVQESNGWTFYKNGKTDLGRTYSYLPTITSNG
KGSGSNWYLTDNGVIQTGVQKWADTYYYFDPSTYLRVDNDYRQSQWGDWYMFGKDGRIATKVYQWAST
YYYFDPSTYLRVDNDYRQSQWGDWYLFGNDGRIQTGVQKWYDTYYYFDPSTYLRVDNDYRQSQWGDWY
MFGPDGRIVSGLYGWKESLYYFTPYLYTKATNQWVSANGKSYWASGSGIITSGLNSINNYILNNNLGHANITF
YDNGQAIPLNITGKYSGTGNGLPNIVIVHETANPNDSIWGEINYEKNHYNDAFVHAFVDNNNIIQISNTDHEA
WGAGYPANGRAVQFEQVEVHNADAFARELSNAAYYTAYIMHKYGFAPSLVSNGNGTLWSHHNVSQYLGG
TDHTDPDGYWYTNAHNFYGTDYTMRDFYELVSLYYGEF
SEQ ID NO: 17
>slpA (LAR_1193)-Coding Sequence
5′/ATGTCGAAGAACAATGCACAAGAATATGTACGCAAAATGGAGCCGCAACGGCAACGATTTGGATTA
AGAAAACTCAGTGTTGGTGTTGCGTCTGTGTTACTAGGAACTACTTTTATGGTCGGAGGTACAGTAGCA
CACGCTAATACTGATAGTACACCGGCGCCAACCGCAGCAGAATCAGTAAGCACTAATGTCGTTAGTCAA
AATGATGCTCAATCACAGGCAGCTATTAGTTCACAAACAAGTGGAAGTCAAGTCGAAATGGTTACTAAC
AGAAATAAAAATGTAATTAAAACTAATGACGTCCAATTACAAAATTTAAATACACCAATTGTAACTTCAC
TTTCAGCTACTATTAATTTAAACTGGACTAATGAAAACGGAGAGCCAGAAAATCTTCCAAGCATTACTTA
TGAAGGTAAAACTGGTGATACTCTTCAAGATGTAGGTAAATACATTCAAGGTTTAATAACTACAGATAAT
TCAAAGTATGAAGTTTCCCCATTAGTATCAACGCCTAAAGATACAGAAGATTATTTAAGTGGAAATACTG
CAGACAGTATCACTGATCCTAGTATGAAATTAGCCTATGACGATTGGCGTAAAAATAAAGAAAAGAATT
ATCCTTTTGCGGGTTACACTGTAGAGATTGATTCATTATCGAATGCTCCATTAACAAATGGAGGTACCTA
CACTATCAATTTGGGAACCGAAACTCGCCTTTATAATGAACCATATTGGGTAATAACTTCACGCACTATTC
ATTATGTAAAATATGGTCTAACGGGTTCTGATAGTGTTGCTTCTCCAGATGTGATCCAGGAAGGTTATTC
AAATGTAACCAATTCTAAAAATAATCCAGTGGTAAAGAACTTTAATTTAGAAAAAGATGGTCATCACTAT
GTGAGTTATGAAACGGTTCAAAGATCGTATAATGTAGCTTCTGGTATTCCTGATGGGATGACAGATAAA
CCCAACTATTACTGA/3′
SEQ ID NO: 18
>slpA (LAR_1193)-Protein Sequence
MSKNNAQEYVRKMEPQRQRFGLRKLSVGVASVLLGTTFMVGGTVAHANTDSTPAPTAAESVSTNVVSQN
DAQSQAAISSQTSGSQVEMVTNRNKNVIKTNDVQLQNLNTPIVTSLSATINLNWTNENGEPENLPSITYEGK
TGDTLQDVGKYIQGLITTDNSKYEVSPLVSTPKDTEDYLSGNTADSITDPSMKLAYDDWRKNKEKNYPFAGYT
VEIDSLSNAPLTNGGTYTINLGTETRLYNEPYWVITSRTIHYVKYGLTGSDSVASPDVIQEGYSNVTNSKNNPV
VKNFNLEKDGHHYVSYETVQRSYNVASGIPDGMTDKPNYY
SEQ ID NO: 19
>apf1 (LAR_0410)-Coding Sequence
5′/ATGATTTCTAAGAAAAACTTTGCTAAAGTATCTGCTACTCTTGGTGCAGTGGCCTTAGGTGTTAGTGC
AACGGCTACTGCTGCTAATGCCGACACTATCTACACCGTGCAAAGTGGTGACACACTTTCAGGTATTTCT
TACAAATTCGCAAAAGACAACAGTATGGTCAATGATCTTGCTAAGAAGAACAATATTCAAGATATTAACA
AGATTTACGTTGGTCAAAAGTTAATCATCAAGAGCGATGGTGAAATTCAAGAATACAACGCTCAAAACG
CAGCTAATGCAAATGTAGCTGACAATAATACTCAAGCTACTCAACAACAAACTGCACAACCTCAACAAGC
TCAAAGTCAAGCTAGCCAAAGTTATACTTCAAATGCTTCAGGTTCAGAAGCTGCTGCTAAGGCTTGGATT
GCCGCTCGTGAATCTGGTGGTAACTACGGTGCTACTAACGGTCAATACATTGGTAAGTACCAATTATCAG
CATCATACCTTAACGGTGACTACTCGGCAGCTAACCAAGAACGGGTTGCTGACCAATACGTTGCAAGTC
GTTACGGTTCATGGCAAAATGCTCAGGCTCACTGGCAAGCTAATGGCTGGTACTAA/3′
SEQ ID NO: 20
>apf1 (LAR_0410)-Protein Sequence
MISKKNFAKVSATLGAVALGVSATATAANADTIYTVQSGDTLSGISYKFAKDNSMVNDLAKKNNIQDINKIYV
GQKLIIKSDGEIQEYNAQNAANANVADNNTQATQQQTAQPQQAQSQASQSYTSNASGSEAAAKAWIAAR
ESGGNYGATNGQYIGKYQLSASYLNGDYSAANQERVADQYVASRYGSWQNAQAHWQANGWY
SEQ ID NO: 21
>cnbP (LAR_0284)-Coding Sequence
ATGAAATTTTGGAAGAAAGCACTATTAACAATTGCAGCCTTAACAGTCGGCACCTCCGCAGGAATTACAA
GCGTTTCTGCCGCTTCATCAGCTGTTAATTCAGAATTAGTTCATAAGGGAGAATTGACAATTGGTCTTGA
GGGAACGTACTCTCCGTACTCTTATCGTAAAAATAACAAATTAACTGGCTTTGAAGTAGATCTTGGTAAA
GCAGTTGCTAAAAAGATGGGCTTAAAAGCTAACTTTGTACCAACTAAATGGGATTCGCTAATTGCCGGTC
TTGGTTCAGGCAAGTTTGATGTAGTAATGAACAACATTACACAGACACCTGAACGGGCCAAGCAATATAA
TTTCTCTACCCCATATATCAAGTCCCGGTTTGCATTAATTGTTCCTACTGATAGTAACATCAAAAGCTTG
AAGGATATTAAAGGCAAGAAGATTATTGCTGGTACGGGAACTAATAATGCGAATGTGGTTAAAAAATATA
AGGGTAACCTTACACCAAATGGCGATTTTGCTAGTTCCTTAGATATGATCAAGCAAGGTCGGGCTGCCGG
GACAGTTAACTCCCGTGAAGCTTGGTACGCTTACAGCAAGAAGAACAGTACTAAGGGTCTCAAGATGATT
GATGTTTCTAGTGAACAAGATCCAGCTAAGATTTCAGCACTTTTTAACAAGAAAGATACTGCTATTCAAT
CTTCCTACAACAAGGCACTGAAGGAACTTCAACAAGATGGAACAGTCAAGAAGCTATCTGAAAAGTACTT
CGGTGCAGATATTACTGAATAA
SEQ ID NO: 22
>cnbP (LAR_0284)-Protein Sequence
MKFWKKALLTIAALTVGTSAGITSVSAASSAVNSELVHKGELTIGLEGTYSPYSYRKNNKLTGFEVDLGKAVAK
KMGLKANFVPTKWDSLIAGLGSGKFDVVMNNITQTPERAKQYNFSTPYIKSRFALIVPTDSNIKSLKDIKGKKI
IAGTGTNNANVVKKYKGNLTPNGDFASSLDMIKQGRAAGTVNSREAWYAYSKKNSTKGLKMIDVSSEQDPAKIS
ALFNKKDTAIQSSYNKALKELQQDGTVKKLSEKYFGADITE

### EXEMPLARY EMBODIMENTS

1. A recombinant microorganism comprising one or more modifications with respect to a corresponding microorganism not comprising the one or more modifications, wherein the one or more modifications reduce, in the recombinant microorganism with respect to the corresponding microorganism, expression and/or activity of one or more proteins expressed by the corresponding microorganism, wherein the one or more proteins comprise any one or more, any two or more, any three or more, any four or more, any five or more, any six or more, or each of a sortase, a sortase-dependent protein, a fibronectin-binding protein, an autolysin, a surface-layer protein, an aggregation-promoting factor, and a collagen-binding protein.

2. The recombinant microorganism of exemplary embodiment 1, wherein the one or more modifications comprise a modification that reduces, in the recombinant microorganism with respect to the corresponding microorganism, expression and/or activity of a sortase expressed by the corresponding microorganism.

3. The recombinant microorganism of any prior exemplary embodiment, wherein the sortase comprises a sequence at least 80% identical to SEQ ID NO:2.

4. The recombinant microorganism of any prior exemplary embodiment, wherein the one or more modifications comprise a modification that reduces, in the recombinant microorganism with respect to the corresponding microorganism, expression and/or activity of a sortase-dependent protein expressed by the corresponding microorganism.

5. The recombinant microorganism of any prior exemplary embodiment, wherein the sortase-dependent protein comprises a sequence at least 80% identical to a sequence selected from the group consisting of SEQ ID NOS:4, 6, 8, 10, and 12.

6. The recombinant microorganism of any prior exemplary embodiment, wherein the one or more modifications comprise a modification that reduces, in the recombinant microorganism with respect to the corresponding microorganism, expression and/or activity of a sortase-dependent protein expressed by the corresponding microorganism that comprises a sequence at least 80% identical to SEQ ID NO:4.

7. The recombinant microorganism of any prior exemplary embodiment, wherein the one or more modifications comprise a modification that reduces, in the recombinant microorganism with respect to the corresponding microorganism, expression and/or activity of a sortase-dependent protein expressed by the corresponding microorganism that comprises a sequence at least 80% identical to SEQ ID NO:6.

8. The recombinant microorganism of any prior exemplary embodiment, wherein the one or more modifications comprise a modification that reduces, in the recombinant microorganism with respect to the corresponding microorganism, expression and/or activity of a sortase-dependent protein expressed by the corresponding microorganism that comprises a sequence at least 80% identical to SEQ ID NO:8.

9. The recombinant microorganism of any prior exemplary embodiment, wherein the one or more modifications comprise a modification that reduces, in the recombinant microorganism with respect to the corresponding microorganism, expression and/or activity of a sortase-dependent protein expressed by the corresponding microorganism that comprises a sequence at least 80% identical to SEQ ID NO:10.

10. The recombinant microorganism of any prior exemplary embodiment, wherein the one or more modifications comprise a modification that reduces, in the recombinant microorganism with respect to the corresponding microorganism, expression and/or activity of a sortase-dependent protein expressed by the corresponding microorganism that comprises a sequence at least 80% identical to SEQ ID NO:12.

11. The recombinant microorganism of any prior exemplary embodiment, wherein the one or more modifications comprise a modification that reduces, in the recombinant microorganism with respect to the corresponding microorganism, expression and/or activity of a fibronectin-binding protein expressed by the corresponding microorganism.

12. The recombinant microorganism of any prior exemplary embodiment, wherein the fibronectin-binding protein comprises a sequence at least 80% identical to SEQ ID NO:14.

13. The recombinant microorganism of any prior exemplary embodiment, wherein the one or more modifications comprise a modification that reduces, in the recombinant microorganism with respect to the corresponding microorganism, expression and/or activity of an autolysin expressed by the corresponding microorganism.

14. The recombinant microorganism of any prior exemplary embodiment, wherein the autolysin comprises a sequence at least 80% identical to SEQ ID NO:16.

15. The recombinant microorganism of any prior exemplary embodiment, wherein the one or more modifications comprise a modification that reduces, in the recombinant microorganism with respect to the corresponding microorganism, expression and/or activity of a surface-layer protein expressed by the corresponding microorganism.

16. The recombinant microorganism of any prior exemplary embodiment, wherein the surface-layer protein comprises a sequence at least 80% identical to SEQ ID NO:18.

17. The recombinant microorganism of any prior exemplary embodiment, wherein the one or more modifications comprise a modification that reduces, in the recombinant microorganism with respect to the corresponding microorganism, expression and/or activity of an aggregation-promoting factor expressed by the corresponding microorganism.

18. The recombinant microorganism of any prior exemplary embodiment, wherein the aggregation-promoting factor comprises a sequence at least 80% identical to SEQ ID NO:20.

19. The recombinant microorganism of any prior exemplary embodiment, wherein the one or more modifications comprise a modification that reduces, in the recombinant microorganism with respect to the corresponding microorganism, expression and/or activity of a collagen-binding protein expressed by the corresponding microorganism.

20. The recombinant microorganism of any prior exemplary embodiment, wherein the collagen-binding protein comprises a sequence at least 80% identical to SEQ ID NO:22.

21. The recombinant microorganism of any prior exemplary embodiment, wherein the recombinant microorganism comprises a recombinant gene configured to express a biologic.

22. The recombinant microorganism of any prior exemplary embodiment, wherein the recombinant microorganism is a member of Lactobacillales (a lactic acid bacterium).

23. The recombinant microorganism of any prior exemplary embodiment, the recombinant microorganism is a member of Limosilactobacillus or Lactobacillus.

24. The recombinant microorganism of any prior exemplary embodiment, wherein the recombinant microorganism is an L. reuteri.

25. The recombinant of any prior exemplary embodiment, wherein the recombinant microorganism exhibits a growth rate during exponential phase of growth no less than 90% of a growth rate exhibited by the corresponding microorganism during exponential phase of growth.

26. A method of administration comprising administering the recombinant microorganism of any prior exemplary embodiment to a subject.

27. The method of exemplary embodiment 26, wherein the administering comprises orally administering the recombinant microorganism to the subject.

28. The method of any one of exemplary embodiments 26-27, wherein the administering introduces the recombinant microorganism to a gastrointestinal tract of the subject.

29. The method of any one of exemplary embodiments 26-28, wherein the recombinant microorganism comprises a recombinant gene configured to express a biologic.

30. The method of exemplary embodiment 29, wherein the administering introduces the biologic to a gastrointestinal tract of the subject.

What is claimed is:

1. A recombinant microorganism comprising one or more modifications with respect to a corresponding microorganism not comprising the one or more modifications, wherein the one or more modifications reduce, in the recombinant microorganism with respect to the corresponding microorganism, expression and/or activity of one or more proteins expressed by the corresponding microorganism, wherein the one or more proteins comprise any one or more, any two or more, any three or more, any four or more, any five or more, any six or more, or each of a sortase, a sortase-dependent protein, a fibronectin-binding protein, an autolysin, a surface-layer protein, an aggregation-promoting factor, and a collagen-binding protein.

2. The recombinant microorganism of claim 1, wherein:
the sortase comprises a sequence at least 80% identical to SEQ ID NO:2;
the sortase-dependent protein comprises a sequence at least 80% identical to a sequence selected from the group consisting of SEQ ID NOS:4, 6, 8, 10, and 12;
the fibronectin-binding protein comprises a sequence at least 80% identical to SEQ ID NO:14;
the autolysin comprises a sequence at least 80% identical to SEQ ID NO:16;
the surface-layer protein comprises a sequence at least 80% identical to SEQ ID NO:18;
the aggregation-promoting factor comprises a sequence at least 80% identical to SEQ ID NO:20; and
the collagen-binding protein comprises a sequence at least 80% identical to SEQ ID NO:22.

3. The recombinant microorganism of claim 1, wherein:
the sortase comprises a sequence at least 95% identical to SEQ ID NO:2;
the sortase-dependent protein comprises a sequence at least 95% identical to a sequence selected from the group consisting of SEQ ID NOS:4, 6, 8, 10, and 12;
the fibronectin-binding protein comprises a sequence at least 95% identical to SEQ ID NO:14;
the autolysin comprises a sequence at least 95% identical to SEQ ID NO:16;
the surface-layer protein comprises a sequence at least 95% identical to SEQ ID NO:18;
the aggregation-promoting factor comprises a sequence at least 95% identical to SEQ ID NO:20; and
the collagen-binding protein comprises a sequence at least 95% identical to SEQ ID NO:22.

4. The recombinant microorganism of claim 1, wherein the recombinant microorganism comprises a recombinant gene configured to express a biologic.

5. The recombinant microorganism of claim 1, wherein the recombinant microorganism is a member of Lactobacillales (a lactic acid bacterium).

6. The recombinant microorganism of claim 1, the recombinant microorganism is a member of Limosilactobacillus or Lactobacillus.

7. The recombinant microorganism of claim 1, wherein the recombinant microorganism is an L. reuteri.

8. The recombinant of claim 1, wherein the recombinant microorganism exhibits a growth rate during exponential phase of growth no less than 90% of a growth rate exhibited by the corresponding microorganism during exponential phase of growth.

9. The recombinant microorganism of claim 1, wherein the one or more proteins comprise any one or more, any two or more, any three or more, any four or more, any five or more, or each of a sortase, a sortase-dependent protein, a fibronectin-binding protein, an autolysin, a surface-layer protein, and an aggregation-promoting factor.

10. The recombinant microorganism of claim 9, wherein:
the sortase comprises a sequence at least 95% identical to SEQ ID NO:2;
the sortase-dependent protein comprises a sequence at least 95% identical to a sequence selected from the group consisting of SEQ ID NOS:4, 6, 8, 10, and 12;
the fibronectin-binding protein comprises a sequence at least 95% identical to SEQ ID NO:14;
the autolysin comprises a sequence at least 95% identical to SEQ ID NO:16;
the surface-layer protein comprises a sequence at least 95% identical to SEQ ID NO:18; and
the aggregation-promoting factor comprises a sequence at least 95% identical to SEQ ID NO:20.

11. The recombinant microorganism of claim 1, wherein the one or more proteins comprise any three or more of a sortase, a sortase-dependent protein, a fibronectin-binding protein, an autolysin, a surface-layer protein, and an aggregation-promoting factor.

12. The recombinant microorganism of claim 11, wherein:
the sortase comprises a sequence at least 95% identical to SEQ ID NO:2;
the sortase-dependent protein comprises a sequence at least 95% identical to a sequence selected from the group consisting of SEQ ID NOS:4, 6, 8, 10, and 12;
the fibronectin-binding protein comprises a sequence at least 95% identical to SEQ ID NO:14;
the autolysin comprises a sequence at least 95% identical to SEQ ID NO:16;
the surface-layer protein comprises a sequence at least 95% identical to SEQ ID NO:18; and
the aggregation-promoting factor comprises a sequence at least 95% identical to SEQ ID NO:20.

13. The recombinant microorganism of claim 1, wherein the one or more proteins comprise each of a sortase, a sortase-dependent protein, a fibronectin-binding protein, an autolysin, a surface-layer protein, and an aggregation-promoting factor.

14. The recombinant microorganism of claim 13, wherein:
the sortase comprises a sequence at least 95% identical to SEQ ID NO:2;
the sortase-dependent protein comprises a sequence at least 95% identical to a sequence selected from the group consisting of SEQ ID NOS:4, 6, 8, 10, and 12;
the fibronectin-binding protein comprises a sequence at least 95% identical to SEQ ID NO:14;
the autolysin comprises a sequence at least 95% identical to SEQ ID NO:16;
the surface-layer protein comprises a sequence at least 95% identical to SEQ ID NO:18; and
the aggregation-promoting factor comprises a sequence at least 95% identical to SEQ ID NO:20.

15. The recombinant microorganism of claim 14, wherein:
the recombinant microorganism is an L. reuteri;
the recombinant microorganism exhibits a growth rate during exponential phase of growth no less than 90% of a growth rate exhibited by the corresponding microorganism during exponential phase of growth; and
the recombinant microorganism comprises a recombinant gene configured to express a biologic.

16. A method of administration comprising administering the recombinant microorganism of any prior claim to a subject.

17. The method of claim 16, wherein the administering comprises orally administering the recombinant microorganism to the subject.

18. The method of claim 16, wherein the administering introduces the recombinant microorganism to a gastrointestinal tract of the subject.

19. The method of claim 16, wherein the recombinant microorganism comprises a recombinant gene configured to express a biologic.

20. The method of claim 16, wherein:
the administering comprises orally administering the recombinant microorganism to the subject;
the administering introduces the recombinant microorganism to a gastrointestinal tract of the subject;
the recombinant microorganism comprises a recombinant gene configured to express a biologic; and
the administering introduces the biologic to a gastrointestinal tract of the subject.


SEQUENCE LISTING (164 sequences)

SEQ ID NO: 1 - DNA, 705 bp, Limosilactobacillus reuteri
  source 1..705: mol_type = genomic DNA
gtgaaaaaagataaaaagcgatcatttgaatggttacggtggacagcggttgttgtactgttactggtatcagttgtcttaatttttaaccaacagattaagtcttacttagtagggagttataaacctgagattactcggcaaacggttcaaagcaaccaaaagaaaaaagcaacctatgattttcaaagtgtcaaagatcttaacttgcaaacagctgccaaggctcgtgcaaataagcaatcgattaataccattggagcaattacggttccggctattaatatgacgattccaattgctaatggagttgataatacaactcttgcgttagcagcaggaacccttcgtccagacatgaagatgggggaaggcaactatgcgttagctggtcataatatggcccatgggagcaaaatcctcttttctccattgtactatcatgctaaggtagggcagatgatttatatcaccaacatggatcgcgtttatgaatataagatttatcaacgtgaattcattgcggcaacccgggttgatgtggtagacaatacgccggaaaagattataactttgattacttgtgacgctaccggggccaatcggttgatgatccgcggtaaatttgttaaatcagagccatttacgaaagcaccacaaaatgtgcaaaagaattttagcgaaaaatatacgacaggtcgttaa

SEQ ID NO: 2 - protein, 234 aa, Limosilactobacillus reuteri
VKKDKKRSFEWLRWTAVVVLLLVSVVLIFNQQIKSYLVGSYKPEITRQTVQSNQKKKATYDFQSVKDLNLQTAAKARANKQSINTIGAITVPAINMTIPIANGVDNTTLALAAGTLRPDMKMGEGNYALAGHNMAHGSKILFSPLYYHAKVGQMIYITNMDRVYEYKIYQREFIAATRVDVVDNTPEKIITLITCDATGANRLMIRGKFVKSEPFTKAPQNVQKNFSEKYTTGR

SEQ ID NO: 3 - DNA, 3669 bp, Limosilactobacillus reuteri
  source 1..3669: mol_type = genomic DNA
atgctatcaagaaaaaattataaggaaactatacgaaaacagacacctacaaaacagtactatactattaagaaattaactgttggggttacttcggtattaattggtctatcctttatgggagaactagaaggggatagcgttcatgcggacacgatgacagcaagcagtgagtcaacaagtgttacgtcgacgactgctcaggatggtttaaaaaaatctccacaactctatttgcaagttactgatacaaataacccaagtacaccattaagtgcttcatccacagggactagtaagaatgttacctcatcagctgcggtacaagtgaagtccgctagtgatgaagaagatagtgattctacactagctaagggagaaaataaatttgctcggtcagcagtaaaagattcagtcactgatgggaaaacaagtacagcagaaattaatccggcaaaattaagcagtcctgctttaataacgcaactcaaccaatccttagctaagagcagtacgagtgatgcagcaaaagctaatgatgagttagaaattaaagcaacagatccgactaattatccaaactgtggcgatgtgtatgggccattatttgaattggatgctagcggacagcttgttaataaagatgaagttatatctcttaaagatatgtatattttccaaatattgaaattagtaaatacaaaagatagtgactttcaatatgtaatattaacaatgaatcgtaaagatactgcagataggtctgtatatctttttgtaactggaagcaattatagtaatgctgttgttgttaaagtaaagccaaatgatacttatgaattaagtaaaactggatatagtgttacttatacagaaccaacaactataaatggacattatgttgatggaactttttatgttacaggaagtacttacgatgatggttttataatgccagattggcaactgcagcaccttcagattatatatagtttaggaaattatgatccaagcaatactgacgcaacatcagtttgtgaaataatgccaagttatgaaaaggtaccggtaattaaatatagtggagtaccttcaaatattagccaacctaaggtttacattaccgggtttacgggtcaagagtttaacgttacagatattattaacaattataagaaagtttttaagggctactatcttcaaaatcctaatgtggcgtccatgggaactctttcccaatttgagaatggtggttattacttaaagacatattatgataatgatggtaatgttgactttaagggcttgtatcatcaaattgatgatcagggaacaatgagtgtgagtgttcttaatgcagataataaaacaattgttggacctgaaaatattcttgctggtaaatcgcataactttaactttaatggtcataactggattgcgcggaatccttatgtcactagttcagctcacgaagtcatattaaagtatgctaagttaggttcagttattcctgttgatgaaaacggaaataaaataaacgatggatggcaatatgttaatgatccagatgatgcttccaaagccactagcccatatgaaaaagcgccagttatcgatggttatgtagctgtaaatccagatgaaacgatcgttcttcctcataacttaagtagtgacacaaagatttattaccgaaagaggattaaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgataaagttacctatagtggtagtgacagcaagacctacgatggtaacccagctaacttcgagccaacgacagttcagtggagtggcttgaaaggactgaacacttcaaccttaacgtccgctgacttcacgtggaatactgcggataagaaggcaccaacggatgccggtaagtacacacttagtttgaatacgaccggagaagcagccttacgtaaggctaacccgaactatgatctcaagacaattagcggtagttacacctacacgattaatccactagggattgtgactgtaaattacaagggctatgataagaaagtctatgatggtcaacctggaacgattaatccgggtaaattaacgtggagtaagttgccagatggtacttcattgaagatgccaacatggagtatagatgatttcgcttgggaaacagctgatggcttagcaccaacggcagtaggaacttatcggattatcttgacggatgctggtaaggctgcactaaagaagattaatccaaattatgacttaagcagtattactggtgtctttacttatgaaattaagccagcacagacaccagaaatcttaggccaaacacctgagcaacaaccaggccaaaatactaatcaatcaggagctgaaaacggctttggttcttctacaaggcctaatgcatcaactaactccaatcttaatcaacttccacagactggtaatgagcattctaatactgcacttgctggtctagcattggctttcttgactgctatgcttggtttgggcaagaagcgtaaacatgattag

SEQ ID NO: 4 - protein, 1222 aa, Limosilactobacillus reuteri
MLSRKNYKETIRKQTPTKQYYTIKKLTVGVTSVLIGLSFMGELEGDSVHADTMTASSESTSVTSTTAQDGLKKSPQLYLQVTDTNNPSTPLSASSTGTSKNVTSSAAVQVKSASDEEDSDSTLAKGENKFARSAVKDSVTDGKTSTAEINPAKLSSPALITQLNQSLAKSSTSDAAKANDELEIKATDPTNYPNCGDVYGPLFELDASGQLVNKDEVISLKDMYIFQILKLVNTKDSDFQYVILTMNRKDTADRSVYLFVTGSNYSNAVVVKVKPNDTYELSKTGYSVTYTEPTTINGHYVDGTFYVTGSTYDDGFIMPDWQLQHLQIIYSLGNYDPSNTDATSVCEIMPSYEKVPVIKYSGVPSNISQPKVYITGFTGQEFNVTDIINNYKKVFKGYYLQNPNVASMGTLSQFENGGYYLKTYYDNDGNVDFKGLYHQIDDQGTMSVSVLNADNKTIVGPENILAGKSHNFNFNGHNWIARNPYVTSSAHEVILKYAKLGSVIPVDENGNKINDGWQYVNDPDDASKATSPYEKAPVIDGYVAVNPDETIVLPHNLSSDTKIYYRKRIKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIDKVTYSGSDSKTYDGNPANFEPTTVQWSGLKGLNTSTLTSADFTWNTADKKAPTDAGKYTLSLNTTGEAALRKANPNYDLKTISGSYTYTINPLGIVTVNYKGYDKKVYDGQPGTINPGKLTWSKLPDGTSLKMPTWSIDDFAWETADGLAPTAVGTYRIILTDAGKAALKKINPNYDLSSITGVFTYEIKPAQTPEILGQTPEQQPGQNTNQSGAENGFGSSTRPNASTNSNLNQLPQTGNEHSNTALAGLALAFLTAMLGLGKKRKHD

SEQ ID NO: 5 - DNA, 2856 bp, Limosilactobacillus reuteri
  source 1..2856: mol_type = genomic DNA
atgagaaattcgaatacaaataattggcgttcaactgctttgattgcgggggcaatcttgacagcaggtgtcgttaccaccacttcaacgacagatgtacacgctgataatagtcaaaataatgcacaagtaagtaatgaccaagttacttacgaccaagttcgggcaactgctgatcagcaacttgcacaattgcaatcagctaatgatagtaaagaagctcaacaagctacggccaacgagcaatcaaatgctgcggatcttgcacaaatcaacgcacaaattgatcaattaaaagctagtcatgctgctctagaacaagaacaagccggtgctattgcacaagcacaagcttctactgctgcttctattgaagcacaaacttcagctgctaatgctgaataccaacaacaaattaacgcacaacaacaaaacgaagccactcaacgtgcagcaaatgatcaacagtacgcacaagctgttagtcaagctgctgaaaatcaacaagctattgctgatatcaatgcacaatatcaacaagctttaagtgatgcagctaatacgcagacaagccaagcacaggctgctgtcaataattacaactcagctgttgctaaggaaaatgatgcttaccaaggtaagctatctgctcaacaagcaactaatgcagaaaatgttaaaaatgctgaagcatctcttgcaaccgcaactgctaatgctaacaagccttatgaagttgcaactactaacgatgttgctgttactggccgagttgctaaaccacaaacagatgttaaggttcctgactatgtagcaaaatacggaattcatctagaagttcgtggctataatggtgctattcctgtatctgattatacttatcctgaatttgctaatacttactatggtgaatatcctgatagtgcagtaattacaaataaggctgctgactcaactccacatgatatttcagagatgccattagatttccaaccaggtctattaagttacgatactaaaaatgatcattcagaaaaagtttctgctgatggattaacagctgcccaagttcaagtattacgggcattagctctttcatgggaaaatggattccgtaacaatgttttccaaaactaccgtgaattttacaatgcggttaataagaatgatggctttgtgaacgttgctccagttgatttagtaagcactgactttgctgataacattgctaaccaagttgttgctaaccggacaaagtacaatgttgataataatagtcatactgttatcagcagtggaatgcctactgaagcaacttatagtagcattattaataatgccacccaaaacttgcaaaagagtttaacaagtgaatttaatggtaagacagtttacactgacgttaatgaaaacttaactactatgcaagctgggacgccaactcttctaaattacgctattaatctctacaactcaatgcagggtatgtactatggtgaattggtaaacccaacacacatcggtggacatgcagttaatcttcttcgtgctggtgttcgcactgtgggaattggcttccaaaagctaactgatgaaatgactgctaagaatggttcaggtgtttctgatcgcaagaacccatcatatgctgtaacatttgatcatgtcggatttaacattgatcacgatgttaacttaactaaagaagttcaaaatcgtaatgtatggggaacttcaactgtagatgcacaattaaatgctattaagactgctcaatatcatgttaataaagtaccggggacaaagacagttacgccaactgctactgatgtgcaaaaagctactgctagcgaacaacaaaaattagctaatgttaaggcacaagcacaacaatctcttgatgcattagctagtgcccaccaagctaacttaaataagcttgaacaacaatataatgatgcgaaggctcaagtaaaagctaactatgatactgcagttactaaggcagctagtgttcgtgatgctgctcttaaggctactggagctgttgaccttcaagcttacaaggctcaattagacagtgcctaccaaaacttagtaaaggctgatcaagtagccgcacaaaagcttgctgctgatcgtgacgctaagatcgctaatattaagaaaactgaaaatgcaaagcttgatgctaagattaatgaacttgttccaagcattgacccacaaatcaagcaattgcaagatcaacaccaagcagttattacccatggtgatgctgttcttgctgaattaaaggctgaaaataaggctgcttataacaaacttgctgaacaattcaacgttcaattagcagctattcaacaacgtgaagatgctaagagagctaatgaagttcggttagcaaataatactgttgttcttcctacagcagtgaaagcagctaatgatggcaatgttgatactgttgctttcccaacggttacaaaccgtctacaagctgaaactactggacgcgttcaaccagtttcagcaccatcgattgtaaagacagctacgcaaacgccaattgcagttgcaccagttgtaacacaaacagctgcagttgctgctactacaaataatggtacacaaacacgggttgcttctactgctgataataaagccactaaagcagacaacaatgaaaaatcagttgtaaaagctgacactgttgctaagaagcaaactcctgctaagaagagtgaatcatcacaaaaggttgatcaatcttctatgtcaattgttgctttagctgccactgcattacttggtacactcggtatcacttacagctcaaagaagcgccacaactaa

SEQ ID NO: 6 - protein, 951 aa, Limosilactobacillus reuteri
MRNSNTNNWRSTALIAGAILTAGVVTTTSTTDVHADNSQNNAQVSNDQVTYDQVRATADQQLAQLQSANDSKEAQQATANEQSNAADLAQINAQIDQLKASHAALEQEQAGAIAQAQASTAASIEAQTSAANAEYQQQINAQQQNEATQRAANDQQYAQAVSQAAENQQAIADINAQYQQALSDAANTQTSQAQAAVNNYNSAVAKENDAYQGKLSAQQATNAENVKNAEASLATATANANKPYEVATTNDVAVTGRVAKPQTDVKVPDYVAKYGIHLEVRGYNGAIPVSDYTYPEFANTYYGEYPDSAVITNKAADSTPHDISEMPLDFQPGLLSYDTKNDHSEKVSADGLTAAQVQVLRALALSWENGFRNNVFQNYREFYNAVNKNDGFVNVAPVDLVSTDFADNIANQVVANRTKYNVDNNSHTVISSGMPTEATYSSIINNATQNLQKSLTSEFNGKTVYTDVNENLTTMQAGTPTLLNYAINLYNSMQGMYYGELVNPTHIGGHAVNLLRAGVRTVGIGFQKLTDEMTAKNGSGVSDRKNPSYAVTFDHVGFNIDHDVNLTKEVQNRNVWGTSTVDAQLNAIKTAQYHVNKVPGTKTVTPTATDVQKATASEQQKLANVKAQAQQSLDALASAHQANLNKLEQQYNDAKAQVKANYDTAVTKAASVRDAALKATGAVDLQAYKAQLDSAYQNLVKADQVAAQKLAADRDAKIANIKKTENAKLDAKINELVPSIDPQIKQLQDQHQAVITHGDAVLAELKAENKAAYNKLAEQFNVQLAAIQQREDAKRANEVRLANNTVVLPTAVKAANDGNVDTVAFPTVTNRLQAETTGRVQPVSAPSIVKTATQTPIAVAPVVTQTAAVAATTNNGTQTRVASTADNKATKADNNEKSVVKADTVAKKQTPAKKSESSQKVDQSSMSIVALAATALLGTLGITYSSKKRHN

SEQ ID NO: 7 - DNA, 2259 bp, Limosilactobacillus reuteri
  source 1..2259: mol_type = genomic DNA
atgaagaataatagttcaaaatattgtttattgttagggacagcattgttaggattatatttccaagctaatagtgttcatgcggatatgactgggacaacagttaatggtgaaactgcccatagcaatgttacgccaatggtccagactaataaggatgaagcaagtactccgcaaacgactactgattggtctgacctagccaaatatcaaaatgacattccagttcagattttaggaatcaatgacctgcatggtgggttagaaaccactggatcagctacgattggagataagacgtattcgaatgccggaacagttgcacgcctagctggtaaccttgatgcggcagaggaaagttttaagaacgctaatccgacaggaacctcaattcgggtagaagccggagatatggttggggcttctccagcaaattctgctcttcttcaagacgaatcaactatgcatgctttagacgcaatgcattttgaaataggaactttgggaaaccatgagtttgatgaaggtttagctgagtatatgcggattgttaatggtggtgaacctactaaacaatataatgaagctgagatggcctatcctcatgtgaaaacagggattaatatcattactgccaatgttgtaaataaatctgatggtcaaatcccatttggaatgcaaccatacttgattaaagaaattcatactagtgatggtaaagttgctcggattggctttattgggattgaaattacttccctaccaattttaaccttatacgataattacaaagattatgatgttttagacgaggctgaaacaattgcaaaatatgatcaaattttacgtaaaaagggtgttaatgcaattgtagttcttgcccatacaggggtttcaacggataaagatggcagcactaaaggtaatgctgttgatatcattaagaagctttaccaaattgaccctgataattctgtcgacctttatgttgccggtcactcccaccaatatgctaatgctactgttggtagtgtaaaattagtgcaagccatttacacaggtaaggcttacgatgatattatcggttacatcgatccaacaactaatgattttgcaccgaatagtctcgtttcgcatgtctttcctgtattatcagaaaaggatgcgcctaatatcaaaacagatgcaaatgttacagcaattgttgaagatgctaacaatagagtagcaccaattattaataagaaaataggggaagctgctaccacaggcgatattcttggacgactacataatacccctactcgtgaaaatgccgttggtgaattagttgtcgatggtcaattatatgccgctcataaggtgggcttaccagctgattttgcgatgactaatacaggcggtgttcgtgcagatctgcatgttaatcctgatcgttccattacatggggaagtgcccaagcggttcaaccatttggtaatattttgcgggtagttgaaatgacaggggcacaaatagttgaagccttgaatcaacaatatgacgaagatcaagcttactacttacaaatttctggacttcattatacttatactgaccaaaacgatcctaaccaaccatataaggtcgttcaagtttatgaccaacataatcaaccgcttgatatgaataagacttacaatgttgttattaatgactttttagcaggtggcggagatggcttttctgcatttaagggtaccaaagttgtcgggattgtcgggcaagatacagacgcgtttattgattatattactgatatgactaatgatggtaaaccaattaccgcaccaacaatgaaccgtaagatttacttgactgctgaacaattagcgaaggctgacgcagattcacagtcacaaacaggaactaatcagaacactcaaaacgatgctaattcccagactgaaggaaatcagcttcaagaagttccgagccaaccggtatctccaacagtaaccttgccaacaacagctggtcaacccgccgaaactgttacattacatgctcaatctaagcaacaaaccgtagctgctaataatcaattaattaatttgacgcctacatcaattaatggccaaaaacaaaaagcaactgaccagcaagcagctttaccacaaactggtaacgatgaagatcttgcattacttcttctcggaaattcattaatggcagcaaccggattgacaattattgatcgcaagcgtaaacatgcttaa

SEQ ID NO: 8 - protein, 752 aa, Limosilactobacillus reuteri
MKNNSSKYCLLLGTALLGLYFQANSVHADMTGTTVNGETAHSNVTPMVQTNKDEASTPQTTTDWSDLAKYQNDIPVQILGINDLHGGLETTGSATIGDKTYSNAGTVARLAGNLDAAEESFKNANPTGTSIRVEAGDMVGASPANSALLQDESTMHALDAMHFEIGTLGNHEFDEGLAEYMRIVNGGEPTKQYNEAEMAYPHVKTGINIITANVVNKSDGQIPFGMQPYLIKEIHTSDGKVARIGFIGIEITSLPILTLYDNYKDYDVLDEAETIAKYDQILRKKGVNAIVVLAHTGVSTDKDGSTKGNAVDIIKKLYQIDPDNSVDLYVAGHSHQYANATVGSVKLVQAIYTGKAYDDIIGYIDPTTNDFAPNSLVSHVFPVLSEKDAPNIKTDANVTAIVEDANNRVAPIINKKIGEAATTGDILGRLHNTPTRENAVGELVVDGQLYAAHKVGLPADFAMTNTGGVRADLHVNPDRSITWGSAQAVQPFGNILRVVEMTGAQIVEALNQQYDEDQAYYLQISGLHYTYTDQNDPNQPYKVVQVYDQHNQPLDMNKTYNVVINDFLAGGGDGFSAFKGTKVVGIVGQDTDAFIDYITDMTNDGKPITAPTMNRKIYLTAEQLAKADADSQSQTGTNQNTQNDANSQTEGNQLQEVPSQPVSPTVTLPTTAGQPAETVTLHAQSKQQTVAANNQLINLTPTSINGQKQKATDQQAALPQTGNDEDLALLLLGNSLMAATGLTIIDRKRKHA

SEQ ID NO: 9 - DNA, 906 bp, Limosilactobacillus reuteri
  source 1..906: mol_type = genomic DNA
atgatagcttttttcgatttaaaaaatgcgaattgcttctttttacgtatatatattaaagagtatataaaagaggagcgagataatatgaaccatttaaaccaccttcatgtattgcgaaacacttgcttagtaggcttaacgacagcgtcaacgatcctttcgctgtcgttaaatattccaccagttttagctgcaactcaagagccaattacgcatcccgtatctaacacaaataatcagaaagaacgtgtccaaatcaaacgtgtaatttattttcatctcttaaatgaagttaaaaaagtggaacaagtgtcttatgctcaccgtgaagtaccgaatgattcaaccaaaaaggcaaattggattattgaaccttttgaagaagttaatatccctgagcaagctggttttagaccatcaatggataagattccaactgttacagaaatagaagatctatcgcaattaaaaaatacgataaatgtttattatcagcccctcaataaagatgaagggccaaaaaaggacagcaaagagaatacgttagcagaaaagactaggggtgagcaagtaaaggagaataaaaaagaggaaaaacaagattcgcaggataattaccccgtggtagaagatctgggggatgataatcaggtcacggaaaattatggggaggtaaataatacaagaaagcagcaaaatttgcatcaacgggtaacagggattaataatcgactaaatcgttcacatcgggactcacgtaaaaacgataactctgaccaacaaatacttccacagacgggaaatgagaccgataatttaacgacttttcttggtttggggataacggtaatggtcgcggggatgagtcttttttcgttaaagaaagcgcacaaaaataaataa

SEQ ID NO: 10 - protein, 301 aa, Limosilactobacillus reuteri
MIAFFDLKNANCFFLRIYIKEYIKEERDNMNHLNHLHVLRNTCLVGLTTASTILSLSLNIPPVLAATQEPITHPVSNTNNQKERVQIKRVIYFHLLNEVKKVEQVSYAHREVPNDSTKKANWIIEPFEEVNIPEQAGFRPSMDKIPTVTEIEDLSQLKNTINVYYQPLNKDEGPKKDSKENTLAEKTRGEQVKENKKEEKQDSQDNYPVVEDLGDDNQVTENYGEVNNTRKQQNLHQRVTGINNRLNRSHRDSRKNDNSDQQILPQTGNETDNLTTFLGLGITVMVAGMSLFSLKKAHKNK

SEQ ID NO: 11 - DNA, 1893 bp, Limosilactobacillus reuteri
  source 1..1893: mol_type = genomic DNA
atgaagaaaagaaaattaaagaagagtttagcaacaactgcgacggtgatggctgttacaacgggggtggcggccattagtaattccgccaaagctgatacagtgcaaaataataaaaatactattcaacaaacgctgcctgatgcgaaccaacaagctcagcagaatgtctgtgcagcccaagacgcggttaataaagccaattttgatgtagcaaccgcaaataatgatcttaatattgctaatcaaaatttggcagacgcggatcaaaatgtcgattctaataagaatcaagttaaacaaacgaaagagcaattatcatctcttgagcaaatgaaagaaaatgctcaacaagttttaactaacgctcatgatgaatattatgaattattgaagcaaccagttgatgtttcatacacggcattttctggtggtggattgagcgtttatgaaagatcaatgcttgattatattacttattataaccaaggaaacttaacttctaagaatcaagcgtataaaaatttccttgcgaatgactatcaaggaaacattgatttattgaaaaagattattgctgaaaacgagagaaccattaattatctgaaaatgggtcgaccatttgaagataatattagaaatcttgagcaagcaagattaacaataaaagaagaactagaagaattatataaggtagtggaagcccaaaaagacgggaaatattataaaccaaaagatgatattaggtgggcgaatatcgattcttatggtttgtcatattctccagtaatgtctaatggatggtatgaggataatccaactgaccaaataagtgacttaattgaaaatcacgaggaagctctaaacaaaattcaacaaagattagccgaattacataataaaatgcagtcggctgtagaaaaggcagaaaaaacatccattgctgctcaagaattacttcaaaagttagaggagatttcacaaccgcttattactgctactcaacaaattaaagatgccgaaagtgcaatgtcacaagctcaacaaaatctgacacttcaagaagaactacttactcaggccaaagataagcgcaatgtgatgcaatctcaagcaatcaatgcgcaaaccaaactcttgcaagcacttctcaataaaacaaattcacaagaaaagttagcgtcagcaaaagagaaacttccggcacaaactgataccttaaagtatagttcgctaatcaaccttgaaccactaacggttgagccaggagtaactcctactcctaaagttacgacagcgattgcggttgaaaatgatgatcatcagaataaagctgttattactttgcctggcgatgaacaagaaaataagctgcctcagggaacaaaggtagtgtggaaagatgatgctaaagttgcgaccgaccttcaacgtcccggaaagcatactgaagatgtcttaattgtttttcctgatggatcggtgattttaactagcgagcaagttactgttaccgcgacagaaaaacaggcagacgctaataaagtgacttcgcctgtacaagaaacggttgatagtacccaagaaaataatatgcgtgtgaacgatctttctacccaggtaatagaagttaatgataataatcaaacctcagcaccagaaatattggtgactccgcaaaccaagctaaataccgtccaaccaaacggagtaataaaagggcagacagctcagaataagattacccagccttcattagctaagtcttttgaggataagacaagaaataatgttttgccacgaaccggagatgaatcttcacttccgattattgtccttggcgctgttatttggcttgcaggaattggggcaacacttaaacggtacgaataa

SEQ ID NO: 12 - protein, 630 aa, Limosilactobacillus reuteri
MKKRKLKKSLATTATVMAVTTGVAAISNSAKADTVQNNKNTIQQTLPDANQQAQQNVCAAQDAVNKANFDVATANNDLNIANQNLADADQNVDSNKNQVKQTKEQLSSLEQMKENAQQVLTNAHDEYYELLKQPVDVSYTAFSGGGLSVYERSMLDYITYYNQGNLTSKNQAYKNFLANDYQGNIDLLKKIIAENERTINYLKMGRPFEDNIRNLEQARLTIKEELEELYKVVEAQKDGKYYKPKDDIRWANIDSYGLSYSPVMSNGWYEDNPTDQISDLIENHEEALNKIQQRLAELHNKMQSAVEKAEKTSIAAQELLQKLEEISQPLITATQQIKDAESAMSQAQQNLTLQEELLTQAKDKRNVMQSQAINAQTKLLQALLNKTNSQEKLASAKEKLPAQTDTLKYSSLINLEPLTVEPGVTPTPKVTTAIAVENDDHQNKAVITLPGDEQENKLPQGTKVVWKDDAKVATDLQRPGKHTEDVLIVFPDGSVILTSEQVTVTATEKQADANKVTSPVQETVDSTQENNMRVNDLSTQVIEVNDNNQTSAPEILVTPQTKLNTVQPNGVIKGQTAQNKITQPSLAKSFEDKTRNNVLPRTGDESSLPIIVLGAVIWLAGIGATLKRYE

SEQ ID NO: 13 - DNA, 1683 bp, Limosilactobacillus reuteri
  source 1..1683: mol_type = genomic DNA
atgtcttttgacggcttgtttactcatgcaattgtccatgagctagatcaaaaattaactactggtcgggtagccaaagttagtcaaccttaccctgcagaattaatcattacgatccgtgctcaccggcacaactacccattactcatttctgctaatccaacatacccacgaatccaaattactgagatcccatacaaaaatccggcggttccaactaattttacaatgacaatgcggaaataccttgaaggtgcaatcgtcaataaaattgaacaagttgataatgatcgaattatcaaaattacctttgacactcgtgatgaattaggagatagtcaacaattagttcttgtgagtgagattatggcccggcatagtaatatttcactcgttaatctcaagacgggtaaaatcattgacaccattaagcatgttggctcagatcaaaatcgggttcgcctgttattacctggtgctacctttgtcatgccaccaaagcaagataaagttaacccttacttgccaaaccaggtctactctgatcttgtaagacaaaccgatgacacagtagaattaagtcatcagttacaggagcattatcaaggatttggcaaagactccgcacgggaattagctgcagaattactgcaaagtgataatttaccagctacctatcagcactttcttaagcactttgaaaatccagagccagtattaatcacccactctaatggaaaaactcaatttgctgtgttccctccattaaatattgatggtgagttgcaacatttcgactccctctctgcccttctcgatgccttctatgctaataaagcagagcaagatcgttccaaagaattagctgggcaagtgttaaaggtattgaagaatgagctaaaaaaagatcggcgaaaagtaaaaaaactgcaacaacaattacaagatgcagcaactgcagatcagtaccgaatttgtggtgagatccttactacctatcttagtaaattaactccaggaatgaaagaaatcgaacttcctaacttttatgatgataataagccactaaaaatcaagcttgctccagaattatcaccatcgcgcaacgcccaaaagtactttactaagtataataagctcaaaacctctgttgagtatgtaaaggaacagctaaaactgacaaatgatgaaatcaaatactttgaaaacattgaaaaccagattaaattagctgctcccgctgatattcaagagatcaaactggaactgcaagaacagggttatattaaaaagaagaaaagcgggaaaaagcaacgaaaagttaaggtaagtgctccagaagaattccatactagtgatggcactacggttttagtaggtaaaaacaacctgcaaaatgaccgtcttagctttaaaattgctaataaaaatgaaatttggttacatgttaaagatatcccaggttcccacgtggtaatccgctcaacaaacccatctgaagatacgattctagaagccgcacagttagcagcatacttctctaaaggtcgcgattcagataatgtgcccgttgattatcttcctgtcaagcgacttcataagccaaacggagctaaacctggctttgttatttttacaggtcagaaaacactttatgtaacaccgcataaactttctaactaa

SEQ ID NO: 14 - protein, 560 aa, Limosilactobacillus reuteri
MSFDGLFTHAIVHELDQKLTTGRVAKVSQPYPAELIITIRAHRHNYPLLISANPTYPRIQITEIPYKNPAVPTNFTMTMRKYLEGAIVNKIEQVDNDRIIKITFDTRDELGDSQQLVLVSEIMARHSNISLVNLKTGKIIDTIKHVGSDQNRVRLLLPGATFVMPPKQDKVNPYLPNQVYSDLVRQTDDTVELSHQLQEHYQGFGKDSARELAAELLQSDNLPATYQHFLKHFENPEPVLITHSNGKTQFAVFPPLNIDGELQHFDSLSALLDAFYANKAEQDRSKELAGQVLKVLKNELKKDRRKVKKLQQQLQDAATADQYRICGEILTTYLSKLTPGMKEIELPNFYDDNKPLKIKLAPELSPSRNAQKYFTKYNKLKTSVEYVKEQLKLTNDEIKYFENIENQIKLAAPADIQEIKLELQEQGYIKKKKSGKKQRKVKVSAPEEFHTSDGTTVLVGKNNLQNDRLSFKIANKNEIWLHVKDIPGSHVVIRSTNPSEDTILEAAQLAAYFSKGRDSDNVPVDYLPVKRLHKPNGAKPGFVIFTGQKTLYVTPHKLSN

SEQ ID NO: 15 - DNA, 1599 bp, Limosilactobacillus reuteri
  source 1..1599: mol_type = genomic DNA
gtgactaataaaaagcattataaattatataagtcggggaaaaattggtgtgtcatggcaattactgcactagcattaacagttagtttaactggggttgctagtgctgatacaacagttgatacagcacaggcagaaacgagtttagcacagagttcagcatctgatgctactcaaatagaatcaaccgatgctaaaattggcgaaagtgagactaatcagtctaatcaaaataatcaacagggaaatattagtactcaacaagccacggatcaaaaagcaaactcagtaacaccaaagtcaacagagatatcaacaccagtaaaagatggttgggtacaagaatctaatggctggactttttataaaaatggtaaaactgatttaggtcgtacttattcatatttaccaacaattacaagtaatggtaaaggcagcggaagcaactggtacttaacggataatggtgtaattcagacaggtgttcaaaaatgggccgatacctattactattttgatccaagcacttaccttcgcgtggataatgactaccgtcaatcccaatggggcgattggtatatgtttggtaaagatggtagaattgctaccaaagtttatcaatgggctagtacgtactattattttgatccaagtacttaccttcgcgtggataatgattaccgtcaatcccaatggggtgactggtatctctttggcaatgatggtcgtattcaaaccggagtccaaaaatggtatgatacttattattactttgatccaagtacttaccttcgagtagacaatgattaccgtcaatcccaatggggggattggtatatgtttggtccagatggtcgaattgtttcaggactttatggctggaaggaaagtttgtattactttactccttacttatataccaaggcaactaaccagtgggtttcagcgaatggtaaaagttattgggcaagtggcagtggaattattacttctggattgaattcaattaacaactatattcttaataataatcttggtcatgctaatattacattttatgataatgggcaggccattcctttgaatattactggaaaatatagtggtacgggtaatggattaccaaatatagttatcgtccatgaaactgctaatcctaatgatagtatttggggtgaaataaattacgaaaagaatcattataatgatgcattcgttcatgcctttgtagataataacaatataattcagatctcaaatactgaccatgaggcttggggtgcaggttatcctgctaatggtcgggccgttcaatttgaacaagttgaggtacataatgcagatgcgtttgctcgggaactttctaacgctgcttactacactgcatatattatgcacaagtatgggtttgcgccttcgttagtatctaatggtaacggaactctatggtctcatcataatgtatctcaatatttaggtggtactgatcatacagatccggatggttattggtacacaaatgcccacaatttctatggtactgattatacaatgcgtgatttttatgagttggtaagcctatattatggtgaattctaa

SEQ ID NO: 16 - protein, 532 aa, Limosilactobacillus reuteri
VTNKKHYKLYKSGKNWCVMAITALALTVSLTGVASADTTVDTAQAETSLAQSSASDATQIESTDAKIGESETNQSNQNNQQGNISTQQATDQKANSVTPKSTEISTPVKDGWVQESNGWTFYKNGKTDLGRTYSYLPTITSNGKGSGSNWYLTDNGVIQTGVQKWADTYYYFDPSTYLRVDNDYRQSQWGDWYMFGKDGRIATKVYQWASTYYYFDPSTYLRVDNDYRQSQWGDWYLFGNDGRIQTGVQKWYDTYYYFDPSTYLRVDNDYRQSQWGDWYMFGPDGRIVSGLYGWKESLYYFTPYLYTKATNQWVSANGKSYWASGSGIITSGLNSINNYILNNNLGHANITFYDNGQAIPLNITGKYSGTGNGLPNIVIVHETANPNDSIWGEINYEKNHYNDAFVHAFVDNNNIIQISNTDHEAWGAGYPANGRAVQFEQVEVHNADAFARELSNAAYYTAYIMHKYGFAPSLVSNGNGTLWSHHNVSQYLGGTDHTDPDGYWYTNAHNFYGTDYTMRDFYELVSLYYGEF

SEQ ID NO: 17 - DNA, 987 bp, Limosilactobacillus reuteri
  source 1..987: mol_type = genomic DNA
atgtcgaagaacaatgcacaagaatatgtacgcaaaatggagccgcaacggcaacgatttggattaagaaaactcagtgttggtgttgcgtctgtgttactaggaactacttttatggtcggaggtacagtagcacacgctaatactgatagtacaccggcgccaaccgcagcagaatcagtaagcactaatgtcgttagtcaaaatgatgctcaatcacaggcagctattagttcacaaacaagtggaagtcaagtcgaaatggttactaacagaaataaaaatgtaattaaaactaatgacgtccaattacaaaatttaaatacaccaattgtaacttcactttcagctactattaatttaaactggactaatgaaaacggagagccagaaaatcttccaagcattacttatgaaggtaaaactggtgatactcttcaagatgtaggtaaatacattcaaggtttaataactacagataattcaaagtatgaagtttccccattagtatcaacgcctaaagatacagaagattatttaagtggaaatactgcagacagtatcactgatcctagtatgaaattagcctatgacgattggcgtaaaaataaagaaaagaattatccttttgcgggttacactgtagagattgattcattatcgaatgctccattaacaaatggaggtacctacactatcaatttgggaaccgaaactcgcctttataatgaaccatattgggtaataacttcacgcactattcattatgtaaaatatggtctaacgggttctgatagtgttgcttctccagatgtgatccaggaaggttattcaaatgtaaccaattctaaaaataatccagtggtaaagaactttaatttagaaaaagatggtcatcactatgtgagttatgaaacggttcaaagatcgtataatgtagcttctggtattcctgatgggatgacagataaacccaactattactga

SEQ ID NO: 18 - protein, 328 aa, Limosilactobacillus reuteri
MSKNNAQEYVRKMEPQRQRFGLRKLSVGVASVLLGTTFMVGGTVAHANTDSTPAPTAAESVSTNVVSQNDAQSQAAISSQTSGSQVEMVTNRNKNVIKTNDVQLQNLNTPIVTSLSATINLNWTNENGEPENLPSITYEGKTGDTLQDVGKYIQGLITTDNSKYEVSPLVSTPKDTEDYLSGNTADSITDPSMKLAYDDWRKNKEKNYPFAGYTVEIDSLSNAPLTNGGTYTINLGTETRLYNEPYWVITSRTIHYVKYGLTGSDSVASPDVIQEGYSNVTNSKNNPVVKNFNLEKDGHHYVSYETVQRSYNVASGIPDGMTDKPNYY

SEQ ID NO: 19 - DNA, 612 bp, Limosilactobacillus reuteri
  source 1..612: mol_type = genomic DNA
atgatttctaagaaaaactttgctaaagtatctgctactcttggtgcagtggccttaggtgttagtgcaacggctactgctgctaatgccgacactatctacaccgtgcaaagtggtgacacactttcaggtatttcttacaaattcgcaaaagacaacagtatggtcaatgatcttgctaagaagaacaatattcaagatattaacaagatttacgttggtcaaaagttaatcatcaagagcgatggtgaaattcaagaatacaacgctcaaaacgcagctaatgcaaatgtagctgacaataatactcaagctactcaacaacaaactgcacaacctcaacaagctcaaagtcaagctagccaaagttatacttcaaatgcttcaggttcagaagctgctgctaaggcttggattgccgctcgtgaatctggtggtaactacggtgctactaacggtcaatacattggtaagtaccaattatcagcatcataccttaacggtgactactcggcagctaaccaagaacgggttgctgaccaatacgttgcaagtcgttacggttcatggcaaaatgctcaggctcactggcaagctaatggctggtactaa

SEQ ID NO: 20 - protein, 203 aa, Limosilactobacillus reuteri
MISKKNFAKVSATLGAVALGVSATATAANADTIYTVQSGDTLSGISYKFAKDNSMVNDLAKKNNIQDINKIYVGQKLIIKSDGEIQEYNAQNAANANVADNNTQATQQQTAQPQQAQSQASQSYTSNASGSEAAAKAWIAARESGGNYGATNGQYIGKYQLSASYLNGDYSAANQERVADQYVASRYGSWQNAQAHWQANGWY

SEQ ID NO: 21 - DNA, 792 bp, Limosilactobacillus reuteri
  source 1..792: mol_type = genomic DNA
atgaaattttggaagaaagcactattaacaattgcagccttaacagtcggcacctccgcaggaattacaagcgtttctgccgcttcatcagctgttaattcagaattagttcataagggagaattgacaattggtcttgagggaacgtactctccgtactcttatcgtaaaaataacaaattaactggctttgaagtagatcttggtaaagcagttgctaaaaagatgggcttaaaagctaactttgtaccaactaaatgggattcgctaattgccggtcttggttcaggcaagtttgatgtagtaatgaacaacattacacagacacctgaacgggccaagcaatataatttctctaccccatatatcaagtcccggtttgcattaattgttcctactgatagtaacatcaaaagcttgaaggatattaaaggcaagaagattattgctggtacgggaactaataatgcgaatgtggttaaaaaatataagggtaaccttacaccaaatggcgattttgctagttccttagatatgatcaagcaaggtcgggctgccgggacagttaactcccgtgaagcttggtacgcttacagcaagaagaacagtactaagggtctcaagatgattgatgtttctagtgaacaagatccagctaagatttcagcactttttaacaagaaagatactgctattcaatcttcctacaacaaggcactgaaggaacttcaacaagatggaacagtcaagaagctatctgaaaagtacttcggtgcagatattactgaataa

SEQ ID NO: 22 - protein, 263 aa, Limosilactobacillus reuteri
MKFWKKALLTIAALTVGTSAGITSVSAASSAVNSELVHKGELTIGLEGTYSPYSYRKNNKLTGFEVDLGKAVAKKMGLKANFVPTKWDSLIAGLGSGKFDVVMNNITQTPERAKQYNFSTPYIKSRFALIVPTDSNIKSLKDIKGKKIIAGTGTNNANVVKKYKGNLTPNGDFASSLDMIKQGRAAGTVNSREAWYAYSKKNSTKGLKMIDVSSEQDPAKISALFNKKDTAIQSSYNKALKELQQDGTVKKLSEKYFGADITE

SEQ ID NO: 23 - DNA, 27 bp, synthetic construct
  source 1..27: mol_type = other DNA
atgaactttaataaaattgatttagac

SEQ ID NO: 24 - DNA, 23 bp, synthetic construct
  source 1..23: mol_type = other DNA
ttataaaagccagtcattaggcc

SEQ ID NO: 25 - DNA, 27 bp, synthetic construct
  source 1..27: mol_type = other DNA
tcaagaaaaagcattttcaggtatagg

SEQ ID NO: 26 - DNA, 24 bp, synthetic construct
  source 1..24: mol_type = other DNA
tctattattccttggacttcattt

SEQ ID NO: 27 - DNA, 34 bp, synthetic construct
  source 1..34: mol_type = other DNA
ttaaaaattaatctttccagtaataatcaacatc

SEQ ID NO: 28 - DNA, 27 bp, synthetic construct
  source 1..27: mol_type = other DNA
ttaaaatgtaggtttaatttttagggc

SEQ ID NO: 29 - DNA, 71 bp, synthetic construct
  source 1..71: mol_type = other DNA
aagcagtcaaaaagccctaaaaattaaacctacattttaacattatgctttggcagtttattcttgacatg

SEQ ID NO: 30 - DNA, 66 bp, synthetic construct
  source 1..66: mol_type = other DNA
tgcgctgatgttgattattactggaaagattaatttttaatttgattgatagccaaaaagcagcag

SEQ ID NO: 31 - DNA, 26 bp, synthetic construct
  source 1..26: mol_type = other DNA
tctcgctttgattgttctatcgaaag

SEQ ID NO: 32 - DNA, 26 bp, synthetic construct
  source 1..26: mol_type = other DNA
ataaggaagataaatcccataagggc

SEQ ID NO: 33 - DNA, 28 bp, synthetic construct
  source 1..28: mol_type = other DNA
aactttcgccattaatgtgttttatcgg

SEQ ID NO: 34 - DNA, 23 bp, synthetic construct
  source 1..23: mol_type = other DNA
agacagatgacaagccctttagc

SEQ ID NO: 35 - DNA, 25 bp, synthetic construct
  source 1..25: mol_type = other DNA
taatatgagataatgccgactgtac

SEQ ID NO: 36 - DNA, 21 bp, synthetic construct
  source 1..21: mol_type = other DNA
ttcattacatccatgggtgtc

SEQ ID NO: 37 - DNA, 20 bp, synthetic construct
  source 1..20: mol_type = other DNA
tgaatgagtgagtcaacttg

SEQ ID NO: 38 - DNA, 20 bp, synthetic construct
  source 1..20: mol_type = other DNA
taaatatcaccttatttcaa

SEQ ID NO: 39 - DNA, 21 bp, synthetic construct
  source 1..21: mol_type = other DNA
tgatctttgaaccaaaattag

SEQ ID NO: 40 - DNA, 25 bp, synthetic construct
  source 1..25: mol_type = other DNA
agaaaaccgactgtaaaaagtacag

SEQ ID NO: 41 - DNA, 26 bp, synthetic construct
  source 1..26: mol_type = other DNA
atgctatcaagaaaaaattataagga

SEQ ID NO: 42 - DNA, 24 bp, synthetic construct
  source 1..24: mol_type = other DNA
ctaatcatgtttacgcttcttgcc

SEQ ID NO: 43 - DNA, 60 bp, synthetic construct
  source 1..60: mol_type = other DNA
gcagcagaaattgaaataaggtgatatttaatgctatcaagaaaaaattataaggaaact

SEQ ID NO: 44 - DNA, 60 bp, synthetic construct
  source 1..60: mol_type = other DNA
gccgactgtactttttacagtcggttttcttgaatgagtgagtcaacttgaattatttgc

SEQ ID NO: 45 - DNA, 60 bp, synthetic construct
  source 1..60: mol_type = other DNA
ggtttgggcaagaagcgtaaacatgattagtgatctttgaaccaaaattagaaaaccaag

SEQ ID NO: 46 - DNA, 21 bp, synthetic construct
  source 1..21: mol_type = other DNA
gtgaaaaaagataaaaagcga

SEQ ID NO: 47 - DNA, 20 bp, synthetic construct
  source 1..20: mol_type = other DNA
ttaacgacctgtcgtatatt

SEQ ID NO: 48 - DNA, 60 bp, synthetic construct
  source 1..60: mol_type = other DNA
gcagcagaaattgaaataaggtgatatttagtgaaaaaagataaaaagcgatcatttgaa

SEQ ID NO: 49 - DNA, 60 bp, synthetic construct
  source 1..60: mol_type = other DNA
tttagcgaaaaatatacgacaggtcgttaatgatctttgaaccaaaattagaaaaccaag

SEQ ID NO: 50 - DNA, 18 bp, synthetic construct
  source 1..18: mol_type = other DNA
atgtcttttgacggcttg

SEQ ID NO: 51 - DNA, 24 bp, synthetic construct
  source 1..24: mol_type = other DNA
ttagttagaaagtttatgcggtgt

SEQ ID NO: 52 - DNA, 60 bp, synthetic construct
  source 1..60: mol_type = other DNA
tgcatgagtaaacaagccgtcaaaagacattaaatatcaccttatttcaatttctgctgc

SEQ ID NO: 53 - DNA, 60 bp, synthetic construct
  source 1..60: mol_type = other DNA
tatgtaacaccgcataaactttctaactaatgatctttgaaccaaaattagaaaaccaag

SEQ ID NO: 54 - DNA, 26 bp, synthetic construct
  source 1..26: mol_type = other DNA
atgaagaataatagttcaaaatattg

SEQ ID NO: 55 - DNA, 18 bp, synthetic construct
  source 1..18: mol_type = other DNA
ttaagcatgtttacgctt

SEQ ID NO: 56 - DNA, 60 bp, synthetic construct
  source 1..60: mol_type = other DNA
gcagcagaaattgaaataaggtgatatttaatgaagaataatagttcaaaatattgttta

SEQ ID NO: 57 - DNA, 60 bp, synthetic construct
  source 1..60: mol_type = other DNA
attattgatcgcaagcgtaaacatgcttaatgatctttgaaccaaaattagaaaaccaag

SEQ ID NO: 58 - DNA, 18 bp, synthetic construct
  source 1..18: mol_type = other DNA
atgaagaaaagaaaatta

SEQ ID NO: 59 - DNA, 17 bp, synthetic construct
  source 1..17: mol_type = other DNA
ttattcgtaccgtttaa

SEQ ID NO: 60 - DNA, 60 bp, synthetic construct
  source 1..60: mol_type = other DNA
gcagcagaaattgaaataaggtgatatttaatgaagaaaagaaaattaaagaagagttta

SEQ ID NO: 61 - DNA, 60 bp, synthetic construct
  source 1..60: mol_type = other DNA
attggggcaacacttaaacggtacgaataatgatctttgaaccaaaattagaaaaccaag

SEQ ID NO: 62 - DNA, 19 bp, synthetic construct
  source 1..19: mol_type = other DNA
atgtcgaagaacaatgcac

SEQ ID NO: 63 - DNA, 24 bp, synthetic construct
  source 1..24: mol_type = other DNA
tcagtaatagttgggtttatctgt

SEQ ID NO: 64 - DNA, 60 bp, synthetic construct
  source 1..60: mol_type = other DNA
gcagcagaaattgaaataaggtgatatttaatgtcgaagaacaatgcacaagaatatgta

SEQ ID NO: 65 - DNA, 60 bp, synthetic construct
  source 1..60: mol_type = other DNA
gggatgacagataaacccaactattactgatgatctttgaaccaaaattagaaaaccaag

SEQ ID NO: 66 - DNA, 20 bp, synthetic construct
  source 1..20: mol_type = other DNA
gtgactaataaaaagcatta

SEQ ID NO: 67 - DNA, 19 bp, synthetic construct
  source 1..19: mol_type = other DNA
ttagaattcaccataatat

SEQ ID NO: 68 - DNA, 60 bp, synthetic construct
  source 1..60: mol_type = other DNA
gcagcagaaattgaaataaggtgatatttagtgactaataaaaagcattataaattatat

SEQ ID NO: 69 - DNA, 60 bp, synthetic construct
  source 1..60: mol_type = other DNA
ttggtaagcctatattatggtgaattctaatgatctttgaaccaaaattagaaaaccaag

SEQ ID NO: 70 - DNA, 22 bp, synthetic construct
  source 1..22: mol_type = other DNA
atgatttctaagaaaaactttg

SEQ ID NO: 71 - DNA, 19 bp, synthetic construct
  source 1..19: mol_type = other DNA
ttagtaccagccattagct

SEQ ID NO: 72 - DNA, 60 bp, synthetic construct
  source 1..60: mol_type = other DNA
gcagcagaaattgaaataaggtgatatttaatgatttctaagaaaaactttgctaaagta

SEQ ID NO: 73 - DNA, 60 bp, synthetic construct
  source 1..60: mol_type = other DNA
gctcactggcaagctaatggctggtactaatgatctttgaaccaaaattagaaaaccaag

SEQ ID NO: 74 - DNA, 26 bp, synthetic construct
  source 1..26: mol_type = other DNA
atgagaaattcgaatacaaataattg

SEQ ID NO: 75 - DNA, 20 bp, synthetic construct
  source 1..20: mol_type = other DNA
ttagttgtggcgcttctttg

SEQ ID NO: 76 - DNA, 60 bp, synthetic construct
  source 1..60: mol_type = other DNA
gcagcagaaattgaaataaggtgatatttaatgagaaattcgaatacaaataattggcgt

SEQ ID NO: 77 - DNA, 60 bp, synthetic construct
  source 1..60: mol_type = other DNA
acttacagctcaaagaagcgccacaactaatgatctttgaaccaaaattagaaaaccaag

SEQ ID NO: 78 - DNA, 80 bp, synthetic construct
  source 1..80: mol_type = other DNA
tcaaaccaccaggaccaagcgctgaaagacgacgctttctgcttaattcacctaatgggttggtttgatccatgaactgg

SEQ ID NO: 79 - DNA, 81 bp, synthetic construct
  source 1..81: mol_type = other DNA
aaacgcgatccatgttggtgatataaatcatctgcccttgtcaagcatgatagtacaatggagaaaagaggattttgctcc

SEQ ID NO: 80 - DNA, 25 bp, synthetic construct
  source 1..25: mol_type = other DNA
tcctaattcgcaaaataagcagagg

SEQ ID NO: 81 - DNA, 27 bp, synthetic construct
  source 1..27: mol_type = other DNA
aatggattacaaatacaggcaaaatcc

SEQ ID NO: 82 - DNA, 28 bp, synthetic construct
  source 1..28: mol_type = other DNA
ttggtgatataaatcatctgcccttgtc

SEQ ID NO: 83 - DNA, 80 bp, synthetic construct
  source 1..80: mol_type = other DNA
cgttaaaataggaaaacctttgcttaggtcaaatcgcaagctttatccgaaaacagatttagtacctgttcctgtccgat

SEQ ID NO: 84 - DNA, 26 bp, synthetic construct
  source 1..26: mol_type = other DNA
gctatttcttagataaagtggctgac

SEQ ID NO: 85 - DNA, 25 bp, synthetic construct
  source 1..25: mol_type = other DNA
tttgcttaggtcaaatcgcaagctt

SEQ ID NO: 86 - DNA, 26 bp, synthetic construct
  source 1..26: mol_type = other DNA
aaaattggaacatggtgtgacatgga

SEQ ID NO: 87 - DNA, 81 bp, synthetic construct
  source 1..81: mol_type = other DNA
acattttctgcattagttgcttgttgagcagatagctttcaccggtaagcatcattttccttagcaacagctgagttgtaa

SEQ ID NO: 88 - DNA, 20 bp, synthetic construct
  source 1..20: mol_type = other DNA
agttcgggcaactgctgatc

SEQ ID NO: 89 - DNA, 25 bp, synthetic construct
  source 1..25: mol_type = other DNA
gcttgttgagcagatagctttcacc

SEQ ID NO: 90 - DNA, 27 bp, synthetic construct
  source 1..27: mol_type = other DNA
taaccgcattgtaaaattcacggtagt

SEQ ID NO: 91 - DNA, 81 bp, synthetic construct
  source 1..81: mol_type = other DNA
catacccacgaatccaaattactgagatcccatacaaatgataggcggttccaactaattttacaatgacaatgcggaaat

SEQ ID NO: 92 - DNA, 27 bp, synthetic construct
  source 1..27: mol_type = other DNA
ggtgattaatactggctctggattttc

SEQ ID NO: 93 - DNA, 26 bp, synthetic construct
  source 1..26: mol_type = other DNA
gacaacatgaatattattagccgccg

SEQ ID NO: 94 - DNA, 15 bp, synthetic construct
  source 1..15: mol_type = other DNA
tggaaccgcctatca

SEQ ID NO: 95 - DNA, 81 bp, synthetic construct
  source 1..81: mol_type = other DNA
aacactatatccagttttacttaattcataagtatcatcctattttactttaacaacaacagcattactataattgcttcc

SEQ ID NO: 96 - DNA, 18 bp, synthetic construct
  source 1..18: mol_type = other DNA
tacaagcccttaaagtca

SEQ ID NO: 97 - DNA, 21 bp, synthetic construct
  source 1..21: mol_type = other DNA
ttgttgttaaagtaaaatagg

SEQ ID NO: 98 - DNA, 17 bp, synthetic construct
  source 1..17: mol_type = other DNA
atgttacctcatcagct

SEQ ID NO: 99 - DNA, 81 bp, synthetic construct
  source 1..81: mol_type = other DNA
ttgatatttggctaggtcagaccaatcagtagtcgtttattacgtacttgcttcatccttattagtctggaccattggcgt

SEQ ID NO: 100 - DNA, 24 bp, synthetic construct
  source 1..24: mol_type = other DNA
tggtagggaagtaatttcaatccc

SEQ ID NO: 101 - DNA, 24 bp, synthetic construct
  source 1..24: mol_type = other DNA
tcactggcaagtactgaatgttgg

SEQ ID NO: 102 - DNA, 28 bp, synthetic construct
  source 1..28: mol_type = other DNA
gtcagaccaatcagtagtcgtttattac

SEQ ID NO: 103 - DNA, 81 bp, synthetic construct
  source 1..81: mol_type = other DNA
aatttttatacgcttgattcttagaagttaagtttcctcatcaataataagtaatataatcaagcattgatctttcataaa

SEQ ID NO: 104 - DNA, 20 bp, synthetic construct
  source 1..20: mol_type = other DNA
tctaacttttgaagtaattc

SEQ ID NO: 105 - DNA, 20 bp, synthetic construct
  source 1..20: mol_type = other DNA
gaagttaagtttcctcatca

SEQ ID NO: 106 - DNA, 18 bp, synthetic construct
  source 1..18: mol_type = other DNA
gactggccttttgtaatt

SEQ ID NO: 107 - DNA, 81 bp, synthetic construct
  source 1..81: mol_type = other DNA
aaagtgaagttacaattggtgtatttaaattttgtaatcatcagtcattagttttaattacatttttatttctgttagtaa

SEQ ID NO: 108 - DNA, 18 bp, synthetic construct
  source 1..18: mol_type = other DNA
actatcagaacccgttag

SEQ ID NO: 109 - DNA, 20 bp, synthetic construct
  source 1..20: mol_type = other DNA
attaaaactaatgactgatg

SEQ ID NO: 110 - DNA, 18 bp, synthetic construct
  source 1..18: mol_type = other DNA
gaatacttgctgactagt

SEQ ID NO: 111 - DNA, 81 bp, synthetic construct
  source 1..81: mol_type = other DNA
ccaaccatcttttactggtgttgatatctctgttgactactacgttactgagtttgctttttgatccgtggcttgttgagt

SEQ ID NO: 112 - DNA, 24 bp, synthetic construct
  source 1..24: mol_type = other DNA
ggattgacggtaatcattgtctac

SEQ ID NO: 113 - DNA, 25 bp, synthetic construct
  source 1..25: mol_type = other DNA
tgttgatatctctgttgactactac

SEQ ID NO: 114 - DNA, 19 bp, synthetic construct
  source 1..19: mol_type = other DNA
ggcttatagccgatgtgca

SEQ ID NO: 115 - DNA, 81 bp, synthetic construct
  source 1..81: mol_type = other DNA
tgcattagctgcgttttgagcgttgtattcttgaatttactactcgctcttgatgattaacttttgaccaacgtaaatctt

SEQ ID NO: 116 - DNA, 20 bp, synthetic construct
  source 1..20: mol_type = other DNA
ggtgctgttacagcttagta

SEQ ID NO: 117 - DNA, 23 bp, synthetic construct
  source 1..23: mol_type = other DNA
gttaatcatcaagagcgagtagt

SEQ ID NO: 118 - DNA, 20 bp, synthetic construct
  source 1..20: mol_type = other DNA
ctcgacctatacctgtcgaa

SEQ ID NO: 119 - DNA, 81 bp, synthetic construct
  source 1..81: mol_type = other DNA
agcgaatcccatttagttggtacaaagttagcttttaattatctctttttagcaactgctttaccaagatctacttcaaag

SEQ ID NO: 120 - DNA, 23 bp, synthetic construct
  source 1..23: mol_type = other DNA
tccgaatgaattatctggcggac

SEQ ID NO: 121 - DNA, 25 bp, synthetic construct
  source 1..25: mol_type = other DNA
gctggatcttgttcactagaaacat

SEQ ID NO: 122 - DNA, 25 bp, synthetic construct
  source 1..25: mol_type = other DNA
taaagcagttgctaaaaagagataa

SEQ ID NO: 123 - DNA, 35 bp, synthetic construct
  source 1..35: mol_type = other DNA
aaacagatcttggtaaagcagttgctaaaaagatg

SEQ ID NO: 124 - DNA, 35 bp, synthetic construct
  source 1..35: mol_type = other DNA
aaaacatctttttagcaactgctttaccaagatct

SEQ ID NO: 125 - DNA, 80 bp, synthetic construct
  source 1..80: mol_type = other DNA
gtttcccaaaacacctatacctgaaaatgcnttttcnnnntcnattattccttggacttcatttactgggtttaacttaa

SEQ ID NO: 126 - DNA, 80 bp, synthetic construct
  source 1..80: mol_type = other DNA
gtttcccaaaacacctatacctgaaaatgctttttcgacgtctattattccttggacttcatttactgggtttaacttaa

SEQ ID NO: 127 - DNA, 80 bp, synthetic construct
  source 1..80: mol_type = other DNA
gtttcccaaaacacctatacctgaaaatgctttttcttgctcgattattccttggacttcatttactgggtttaacttaa

SEQ ID NO: 128 - DNA, 80 bp, synthetic construct
  source 1..80: mol_type = other DNA
gtttcccaaaacacctatacctgaaaatgcgttttctatttctattattccttggacttcatttactgggtttaacttaa

SEQ ID NO: 129 - DNA, 80 bp, synthetic construct
  source 1..80: mol_type = other DNA
gtttcccaaaacacctatacctgaaaatgccttttcgacatctattattccttggacttcatttactgggtttaacttaa

SEQ ID NO: 130 - DNA, 80 bp, synthetic construct
  source 1..80: mol_type = other DNA
gtttcccaaaacacctatacctgaaaatgctttttccttatccattattccttggacttcatttactgggtttaacttaa

SEQ ID NO: 131 - DNA, 80 bp, synthetic construct
  source 1..80: mol_type = other DNA
gtttcccaaaacacctatacctgaaaatgcgttttctatctctattattccttggacttcatttactgggtttaacttaa

SEQ ID NO: 132 - DNA, 80 bp, synthetic construct
  source 1..80: mol_type = other DNA
gtttcccaaaacacctatacctgaaaatgctttttcagcatctattattccttggacttcatttactgggtttaacttaa

SEQ ID NO: 133 - DNA, 80 bp, synthetic construct
  source 1..80: mol_type = other DNA
gtttcccaaaacacctatacctgaaaatgctttttcagtttcaattattccttggacttcatttactgggtttaacttaa

SEQ ID NO: 134 - DNA, 80 bp, synthetic construct
  source 1..80: mol_type = other DNA
gtttcccaaaacacctatacctgaaaatgctttttcgtgttctattattccttggacttcatttactgggtttaacttaa

SEQ ID NO: 135 - DNA, 57 bp, synthetic construct
  source 1..57: mol_type = other DNA
acactctttccctacacgacgctcttccgatctcctgctgtaataatgggtagaagg

SEQ ID NO: 136 - DNA, 59 bp, synthetic construct
  source 1..59: mol_type = other DNA
gtgactggagttcagacgtgtgctcttccgatcttggactcctgtaaagaatgacttca

SEQ ID NO: 137 - DNA, 63 bp, synthetic construct
  source 1..63: mol_type = other DNA
acactctttccctacacgacgctcttccgatctnnnnnncctgctgtaataatgggtagaagg

SEQ ID NO: 138 - DNA, 65 bp, synthetic construct
  source 1..65: mol_type = other DNA
gtgactggagttcagacgtgtgctcttccgatctnnnnnntggactcctgtaaagaatgacttca

SEQ ID NO: 139 - DNA, 60 bp, synthetic construct
  source 1..60: mol_type = other DNA
aatgatacggcgaccaccgagatctacaccggaagaaacactctttccctacacgacgct

SEQ ID NO: 140 - DNA, 55 bp, synthetic construct
  source 1..55: mol_type = other DNA
caagcagaagacggcatacgagatttcttccggtgactggagttcagacgtgtgc

SEQ ID NO: 141 - DNA, 60 bp, synthetic construct
  source 1..60: mol_type = other DNA
aatgatacggcgaccaccgagatctacacgacaccaaacactctttccctacacgacgct

SEQ ID NO: 142 - DNA, 55 bp, synthetic construct
  source 1..55: mol_type = other DNA
caagcagaagacggcatacgagatttggtgtcgtgactggagttcagacgtgtgc

SEQ ID NO: 143 - DNA, 60 bp, synthetic construct
  source 1..60: mol_type = other DNA
aatgatacggcgaccaccgagatctacacacaactggacactctttccctacacgacgct

SEQ ID NO: 144 - DNA, 55 bp, synthetic construct
  source 1..55: mol_type = other DNA
caagcagaagacggcatacgagatccagttgtgtgactggagttcagacgtgtgc

SEQ ID NO: 145 - DNA, 60 bp, synthetic construct
  source 1..60: mol_type = other DNA
aatgatacggcgaccaccgagatctacaccggtactaacactctttccctacacgacgct

SEQ ID NO: 146 - DNA, 55 bp, synthetic construct
  source 1..55: mol_type = other DNA
caagcagaagacggcatacgagattagtaccggtgactggagttcagacgtgtgc

SEQ ID NO: 147 - DNA, 60 bp, synthetic construct
  source 1..60: mol_type = other DNA
aatgatacggcgaccaccgagatctacacactcacacacactctttccctacacgacgct

SEQ ID NO: 148 - DNA, 55 bp, synthetic construct
  source 1..55: mol_type = other DNA
caagcagaagacggcatacgagatgtgtgagtgtgactggagttcagacgtgtgc

SEQ ID NO: 149 - DNA, 60 bp, synthetic construct
  source 1..60: mol_type = other DNA
aatgatacggcgaccaccgagatctacacctagacgaacactctttccctacacgacgct

SEQ ID NO: 150 - DNA, 55 bp, synthetic construct
  source 1..55: mol_type = other DNA
caagcagaagacggcatacgagattcgtctaggtgactggagttcagacgtgtgc

SEQ ID NO: 151 - DNA, 60 bp, synthetic construct
  source 1..60: mol_type = other DNA
aatgatacggcgaccaccgagatctacacataggtcgacactctttccctacacgacgct

SEQ ID NO: 152 - DNA, 55 bp, synthetic construct
  source 1..55: mol_type = other DNA
caagcagaagacggcatacgagatcgacctatgtgactggagttcagacgtgtgc

SEQ ID NO: 153 - DNA, 60 bp, synthetic construct
  source 1..60: mol_type = other DNA
aatgatacggcgaccaccgagatctacactagcagcaacactctttccctacacgacgct

SEQ ID NO: 154 - DNA, 55 bp, synthetic construct
  source 1..55: mol_type = other DNA
caagcagaagacggcatacgagattgctgctagtgactggagttcagacgtgtgc

SEQ ID NO: 155 - DNA, 21 bp, Limosilactobacillus reuteri
  source 1..21: mol_type = genomic DNA
ctgccctaccttagcatgata

SEQ ID NO: 156 - DNA, 21 bp, Limosilactobacillus reuteri
  source 1..21: mol_type = genomic DNA
agtatcatttggctttacttt

SEQ ID NO: 157 - DNA, 21 bp, Limosilactobacillus reuteri
  source 1..21: mol_type = genomic DNA
agcttttaagcccatcttttt

SEQ ID NO: 158 - DNA, 21 bp, Limosilactobacillus reuteri
  source 1..21: mol_type = genomic DNA
tacaaaaatccggcggttcca

SEQ ID NO: 159 - DNA, 21 bp, Limosilactobacillus reuteri
  source 1..21: mol_type = genomic DNA
ttgtaattggacgtcattagt

SEQ ID NO: 160 - DNA, 21 bp, Limosilactobacillus reuteri
  source 1..21: mol_type = genomic DNA
ttgaatttcaccatcgctctt

SEQ ID NO: 161 - DNA, 21 bp, Limosilactobacillus reuteri
  source 1..21: mol_type = genomic DNA
tgttgactttggtgttactga

SEQ ID NO: 162 - DNA, 21 bp, Limosilactobacillus reuteri
  source 1..21: mol_type = genomic DNA
tagcttaccttggtaagcatc

SEQ ID NO: 163 - DNA, 21 bp, Limosilactobacillus reuteri
  source 1..21: mol_type = genomic DNA
agtcgtttgcggagtacttgc

SEQ ID NO: 164 - DNA, 21 bp, Limosilactobacillus reuteri
  source 1..21: mol_type = genomic DNA
taagtttccttggttataata